SEQUENCE LISTING (276 sequences)

SEQ ID NO: 1 - DNA, 100 bp, Artificial Sequence
  synthesized
gtccccattcctgtttcgtttgcacagaggggttttctggtgcgtcctggtccaccatggccaaaccaacaagcaaagattcaggcttgaaggagaagtt

SEQ ID NO: 2 - DNA, 100 bp, Artificial Sequence
  synthesized
tccaccatggccaaaccaacaagcaaagattcaggcttgaaggagaagtttaagattctgttgggactgggaacaccgaggccaaatcccaggtctgcag

SEQ ID NO: 3 - DNA, 100 bp, Artificial Sequence
  synthesized
taagattctgttgggactgggaacaccgaggccaaatcccaggtctgcagagggtaaacagacggagtttatcatcaccgcggaaatactgagagtgagt

SEQ ID NO: 4 - DNA, 100 bp, Artificial Sequence
  synthesized
agggtaaacagacggagtttatcatcaccgcggaaatactgagagtgagtgagctacctgtgtctttgctaggctagagggaaatgcagagaaggctggg

SEQ ID NO: 5 - DNA, 100 bp, Artificial Sequence
  synthesized
ttaaggagaccgtggcctgagcactggcccctttttcttctttcatctctctccaggaactgagcatggaatgtggcctcaacaatcgcatccggatgat

SEQ ID NO: 6 - DNA, 100 bp, Artificial Sequence
  synthesized
ctccaggaactgagcatggaatgtggcctcaacaatcgcatccggatgatagggcagatttgtgaagtcgcaaaaaccaagaaatttgaagaggtaggtt

SEQ ID NO: 7 - DNA, 100 bp, Artificial Sequence
  synthesized
agggcagatttgtgaagtcgcaaaaaccaagaaatttgaagaggtaggtttatccagttgagctactagagagaggcacgtagactattcagagcctgag

SEQ ID NO: 8 - DNA, 100 bp, Artificial Sequence
  synthesized
tgctccagttgccggggccagggttcttggagagcacatcctcaccgctgtcccctctgctggtgacagcacgcagtggaagcactctggaaggcggtcg

SEQ ID NO: 9 - DNA, 100 bp, Artificial Sequence
  synthesized
tcccctctgctggtgacagcacgcagtggaagcactctggaaggcggtcgcggatctgttgcagccggagcggccgctggaggcccggcacgcggtgctg

SEQ ID NO: 10 - DNA, 100 bp, Artificial Sequence
  synthesized
cggatctgttgcagccggagcggccgctggaggcccggcacgcggtgctggctctgctgaaggccatcgtgcaggggcaggtaaggcccagggcgacgct

SEQ ID NO: 11 - DNA, 100 bp, Artificial Sequence
  synthesized
gctctgctgaaggccatcgtgcaggggcaggtaaggcccagggcgacgctgggatgggtgacgtcaggctgcccactgactgtcctgtccctgctgggcc

SEQ ID NO: 12 - DNA, 100 bp, Artificial Sequence
  synthesized
ccgtgtgggcgacgctggcaggctctgctgatcctgtggcttttgtctttagggcgagcgtttgggggtcctcagagccctcttctttaaggtcatcaag

SEQ ID NO: 13 - DNA, 100 bp, Artificial Sequence
  synthesized
agggcgagcgtttgggggtcctcagagccctcttctttaaggtcatcaaggattacccttccaacgaagaccttcacgaaaggctggaggttttcaaggc

SEQ ID NO: 14 - DNA, 100 bp, Artificial Sequence
  synthesized
gattacccttccaacgaagaccttcacgaaaggctggaggttttcaaggccctcacagacaatgggagacacatcacctacttggaggaagagctgggtg

SEQ ID NO: 15 - DNA, 100 bp, Artificial Sequence
  synthesized
cctcacagacaatgggagacacatcacctacttggaggaagagctgggtgggtgccaccttgggttggaggtttctctggccttgacgatcaagtgtaac

SEQ ID NO: 16 - DNA, 100 bp, Artificial Sequence
  synthesized
ggagggggaggtgagtgggagatgtagattcggcgtcctcgcaaactgccgccgcttctcccccagctgactttgtcctgcagtggatggatgttggctt

SEQ ID NO: 17 - DNA, 100 bp, Artificial Sequence
  synthesized
gccgcttctcccccagctgactttgtcctgcagtggatggatgttggcttgtcctcggaattccttctggtgctggtgaacttggtcaaattcaatagct

SEQ ID NO: 18 - DNA, 100 bp, Artificial Sequence
  synthesized
gtcctcggaattccttctggtgctggtgaacttggtcaaattcaatagctgttacctcgacgagtacatcgcaaggatggttcagtaagaaaagaattga

SEQ ID NO: 19 - DNA, 100 bp, Artificial Sequence
  synthesized
gttacctcgacgagtacatcgcaaggatggttcagtaagaaaagaattgagatcctgttctgataatggtcctaagttcagctccgcagtgaataaagtt

SEQ ID NO: 20 - DNA, 100 bp, Artificial Sequence
  synthesized
ctcggccatccaggcagtgctgccgggactgagctcggtgctccctgcaggatgatctgtctgctgtgcgtccggaccgcgtcctctgtggacatagagg

SEQ ID NO: 21 - DNA, 100 bp, Artificial Sequence
  synthesized
gatgatctgtctgctgtgcgtccggaccgcgtcctctgtggacatagaggtcagtgcctcccctccccagggccggcccatttcaccctggtttctggga

SEQ ID NO: 22 - DNA, 100 bp, Artificial Sequence
  synthesized
tgtcctctcctgtggggaggagctggggtaggacgggcgtgagccgtctccctctccaccaggtctccctgcaggtgctggacgccgtggtctgctacaa

SEQ ID NO: 23 - DNA, 100 bp, Artificial Sequence
  synthesized
cctctccaccaggtctccctgcaggtgctggacgccgtggtctgctacaactgcctgccggctgagagcctcccgctgttcatcgttaccctctgtcgca

SEQ ID NO: 24 - DNA, 100 bp, Artificial Sequence
  synthesized
ctgcctgccggctgagagcctcccgctgttcatcgttaccctctgtcgcaccatcaacgtcaaggagctctgcgagccttgctggaaggtggggtttctg

SEQ ID NO: 25 - DNA, 100 bp, Artificial Sequence
  synthesized
ccatcaacgtcaaggagctctgcgagccttgctggaaggtggggtttctgaaactgctctggaaggttcctgagagcacatggatgggacaagggccatc

SEQ ID NO: 26 - DNA, 100 bp, Artificial Sequence
  synthesized
cgggactggggctgggggcagggcttatgcctgccagcccctgacacgcattgtgtctcgcagctgatgcggaacctccttggcacccacctgggccaca

SEQ ID NO: 27 - DNA, 100 bp, Artificial Sequence
  synthesized
ttgtgtctcgcagctgatgcggaacctccttggcacccacctgggccacagcgccatctacaacatgtgccacctcatggaggacaggtgagtgtggtgg

SEQ ID NO: 28 - DNA, 100 bp, Artificial Sequence
  synthesized
gcgccatctacaacatgtgccacctcatggaggacaggtgagtgtggtgggtggggcgcagggcagtggaggccagcacagccctcggggcagctccagt

SEQ ID NO: 29 - DNA, 100 bp, Artificial Sequence
  synthesized
ccatggcggaccctgggacagggccctgctcacattccgtctctctggggaacacttttagagcctacatggaggacgcgcccctgctgagaggagccgt

SEQ ID NO: 30 - DNA, 100 bp, Artificial Sequence
  synthesized
aacacttttagagcctacatggaggacgcgcccctgctgagaggagccgtgttttttgtgggcatggctctctggggagcccaccggctctattctctca

SEQ ID NO: 31 - DNA, 100 bp, Artificial Sequence
  synthesized
gttttttgtgggcatggctctctggggagcccaccggctctattctctcaggaactcgccgacatctgtgttgccatcattttaccaggtaaggcggttt

SEQ ID NO: 32 - DNA, 100 bp, Artificial Sequence
  synthesized
ggaactcgccgacatctgtgttgccatcattttaccaggtaaggcggtttctgtgtgcagtgagctggcaggaacgggagagctcccctcacgcctgccc

SEQ ID NO: 33 - DNA, 100 bp, Artificial Sequence
  synthesized
cacagcaagcaagcagctctgaccctgtgtgctggccgggctcgtgttccaggccatggcatgtccgaacgaggtggtgtcctatgagatcgtcctgtcc

SEQ ID NO: 34 - DNA, 100 bp, Artificial Sequence
  synthesized
aggccatggcatgtccgaacgaggtggtgtcctatgagatcgtcctgtccatcaccaggctcatcaagaagtataggaaggagctccaggtggtggcgtg

SEQ ID NO: 35 - DNA, 100 bp, Artificial Sequence
  synthesized
atcaccaggctcatcaagaagtataggaaggagctccaggtggtggcgtgggacattctgctgaacatcatcgaacggctccttcagcagctccaggtgg

SEQ ID NO: 36 - DNA, 100 bp, Artificial Sequence
  synthesized
ggacattctgctgaacatcatcgaacggctccttcagcagctccaggtggggtgggggcaggagctccggggagcaccgggaacccagacaggcaggctc

SEQ ID NO: 37 - DNA, 100 bp, Artificial Sequence
  synthesized
gccaagtccatgtggggagtggaagtcagcctgtgtcatcgtgcctggtactgcagaccttggacagcccggagctcaggaccatcgtccatgacctgtt

SEQ ID NO: 38 - DNA, 100 bp, Artificial Sequence
  synthesized
ctgcagaccttggacagcccggagctcaggaccatcgtccatgacctgttgaccacggtggaggagctgtgtgaccagaacgagttccacgggtctcagg

SEQ ID NO: 39 - DNA, 100 bp, Artificial Sequence
  synthesized
gaccacggtggaggagctgtgtgaccagaacgagttccacgggtctcaggagagatactttgaactggtggagagatgtgcggaccagaggcctgtgaga

SEQ ID NO: 40 - DNA, 100 bp, Artificial Sequence
  synthesized
agagatactttgaactggtggagagatgtgcggaccagaggcctgtgagaccccctcctgggtggggcctttgggctttggctggtggggaggggccggg

SEQ ID NO: 41 - DNA, 100 bp, Artificial Sequence
  synthesized
gaccagcagcccagtgtggagaaggagagcgccggaggggcagaggggcaacaccggctcttcttttgacaggagtcctccctcctgaacctgatctcct

SEQ ID NO: 42 - DNA, 100 bp, Artificial Sequence
  synthesized
acaccggctcttcttttgacaggagtcctccctcctgaacctgatctcctatagagcgcagtccatccacccggccaaggacggctggattcagaacctg

SEQ ID NO: 43 - DNA, 100 bp, Artificial Sequence
  synthesized
atagagcgcagtccatccacccggccaaggacggctggattcagaacctgcaggcgctgatggagagattcttcaggtagggggtcctctgtagccttgc

SEQ ID NO: 44 - DNA, 100 bp, Artificial Sequence
  synthesized
caggcgctgatggagagattcttcaggtagggggtcctctgtagccttgcctggcacctggagcctggccctgtctctgtctggggcccacccgggctgg

SEQ ID NO: 45 - DNA, 100 bp, Artificial Sequence
  synthesized
ctggtgtggggctgtggccgggcactccccacccgccccagcaggctgccgtcccgcaggagcgagtcccgaggcgccgtgcgcatcaaggtgctggacg

SEQ ID NO: 46 - DNA, 100 bp, Artificial Sequence
  synthesized
gtcccgcaggagcgagtcccgaggcgccgtgcgcatcaaggtgctggacgtgctgtcctttgtgctgctcatcaacaggcagttctatgaggtgcgtgtc

SEQ ID NO: 47 - DNA, 100 bp, Artificial Sequence
  synthesized
tgctgtcctttgtgctgctcatcaacaggcagttctatgaggtgcgtgtccaggcggccgcagctgggggctcagggctatttctccgtgggcgggctgt

SEQ ID NO: 48 - DNA, 100 bp, Artificial Sequence
  synthesized
ggaattggaagtgtcacgagatgtggccctcgttgggctggcgctcattggcctcccttgtgcctgtgcaggaggagctgattaactcagtggtcatctc

SEQ ID NO: 49 - DNA, 100 bp, Artificial Sequence
  synthesized
gcctcccttgtgcctgtgcaggaggagctgattaactcagtggtcatctcgcagctctcccacatccccgaggataaagaccaccaggtccgaaagctgg

SEQ ID NO: 50 - DNA, 100 bp, Artificial Sequence
  synthesized
gcagctctcccacatccccgaggataaagaccaccaggtccgaaagctggccacccagttgctggtggacctggcagagggctgccacacacaccacttc

SEQ ID NO: 51 - DNA, 100 bp, Artificial Sequence
  synthesized
ccacccagttgctggtggacctggcagagggctgccacacacaccacttcaacagcctgctggacatcatcgagaaggtgagagccgttgtacccggggc

SEQ ID NO: 52 - DNA, 100 bp, Artificial Sequence
  synthesized
aacagcctgctggacatcatcgagaaggtgagagccgttgtacccggggccgggtgctagcgtgccagagctccgtgggcagcaatggcctctgggccct

SEQ ID NO: 53 - DNA, 100 bp, Artificial Sequence
  synthesized
agtgttctcacggctgctgactcagaaccatgagcctgtgtgtaagtcctggccttctcttcaaaggtgatggcccgctccctctccccacccccggagc

SEQ ID NO: 54 - DNA, 100 bp, Artificial Sequence
  synthesized
ggccttctcttcaaaggtgatggcccgctccctctccccacccccggagctggaagaaagggatgtggccgcatactcggcctccttggaggatgtgaag

SEQ ID NO: 55 - DNA, 100 bp, Artificial Sequence
  synthesized
tggaagaaagggatgtggccgcatactcggcctccttggaggatgtgaagacagccgtcctggggcttctggtcatccttcaggtgggtgttctgcacga

SEQ ID NO: 56 - DNA, 100 bp, Artificial Sequence
  synthesized
acagccgtcctggggcttctggtcatccttcaggtgggtgttctgcacgaggcctctgctcccggggcgcgcatggctagcgtccaccagctgcatctgc

SEQ ID NO: 57 - DNA, 100 bp, Artificial Sequence
  synthesized
gtgctgtcttaggactgcgttttcacctcctgcgccgtggtgagctgcgtcctctctctgcagaccaagctgtacaccctgcctgcaagccacgccacgc

SEQ ID NO: 58 - DNA, 100 bp, Artificial Sequence
  synthesized
cctctctctgcagaccaagctgtacaccctgcctgcaagccacgccacgcgtgtgtatgagatgctggtcagccacattcagctccactacaagcacagc

SEQ ID NO: 59 - DNA, 100 bp, Artificial Sequence
  synthesized
gtgtgtatgagatgctggtcagccacattcagctccactacaagcacagctacaccctgccaatcgcgagcagcatccggctgcaggtatggtggctggg

SEQ ID NO: 60 - DNA, 100 bp, Artificial Sequence
  synthesized
tacaccctgccaatcgcgagcagcatccggctgcaggtatggtggctggggttgcgcagccagttcctgggggcccagccaggtatccccgtctcggcag

SEQ ID NO: 61 - DNA, 100 bp, Artificial Sequence
  synthesized
gcaggtgggacgccgcctgtcctgggcctgcacgagcttggctctggctttcaccatcctcttcctgacaggcctttgacttcctgttgctgctgcgggc

SEQ ID NO: 62 - DNA, 100 bp, Artificial Sequence
  synthesized
tcaccatcctcttcctgacaggcctttgacttcctgttgctgctgcgggccgactcactgcaccgcctgggcctgcccaacaaggatggagtcgtgcggt

SEQ ID NO: 63 - DNA, 100 bp, Artificial Sequence
  synthesized
cgactcactgcaccgcctgggcctgcccaacaaggatggagtcgtgcggttcagcccctactgcgtctgcgactacatgtacgcgggacctcgcccacgg

SEQ ID NO: 64 - DNA, 100 bp, Artificial Sequence
  synthesized
tcagcccctactgcgtctgcgactacatgtacgcgggacctcgcccacggcccatgaggctcagggcgtcagaggcgctggggctgtggtggcgctgttt

SEQ ID NO: 65 - DNA, 100 bp, Artificial Sequence
  synthesized
gggttgggaagagccaagtctgttccgttcctgctgcggggacttggcctcagctgcttctcttgcttctgcagggagccagagagaggctctgagaaga

SEQ ID NO: 66 - DNA, 100 bp, Artificial Sequence
  synthesized
cagctgcttctcttgcttctgcagggagccagagagaggctctgagaagaagaccagcggccccctttctcctcccacagggcctcctggcccggcgcct

SEQ ID NO: 67 - DNA, 100 bp, Artificial Sequence
  synthesized
agaccagcggccccctttctcctcccacagggcctcctggcccggcgcctgcaggccccgccgtgcggctggggtccgtgccctactccctgctcttccg

SEQ ID NO: 68 - DNA, 100 bp, Artificial Sequence
  synthesized
gcaggccccgccgtgcggctggggtccgtgccctactccctgctcttccgcgtcctgctgcagtgcttgaagcaggtgagtggggccgggcagggaccat

SEQ ID NO: 69 - DNA, 100 bp, Artificial Sequence
  synthesized
cgtcctgctgcagtgcttgaagcaggtgagtggggccgggcagggaccatccgtcccacgttgggccaggaggacagggagctgccacctgcctgctggg

SEQ ID NO: 70 - DNA, 100 bp, Artificial Sequence
  synthesized
ctagcttccgcctctgtctctagggtccagaaggccctgtcctgacgcctcctctcctcgcaggagtctgactggaaggtgctgaagctggttctgggca

SEQ ID NO: 71 - DNA, 100 bp, Artificial Sequence
  synthesized
cctctcctcgcaggagtctgactggaaggtgctgaagctggttctgggcaggctgcctgagtccctgcgctataaagtgctcatctttacttccccttgc

SEQ ID NO: 72 - DNA, 100 bp, Artificial Sequence
  synthesized
ggctgcctgagtccctgcgctataaagtgctcatctttacttccccttgcagtgtggaccagctgtgctctgctctctgctccatggtaccatggccggc

SEQ ID NO: 73 - DNA, 100 bp, Artificial Sequence
  synthesized
agtgtggaccagctgtgctctgctctctgctccatggtaccatggccggcctggggttggggtgggggacccagtagggtttttccccaaaagactgcga

SEQ ID NO: 74 - DNA, 100 bp, Artificial Sequence
  synthesized
ggctaccccgtgacctggccgctggggagaggtttcatgcctggatttggtcatcagctttcaggcccaaagacactggagcggctccgaggcgccccag

SEQ ID NO: 75 - DNA, 100 bp, Artificial Sequence
  synthesized
tcatcagctttcaggcccaaagacactggagcggctccgaggcgccccagaaggcttctccagaactgacttgcacctggccgtggttccagtgctgaca

SEQ ID NO: 76 - DNA, 100 bp, Artificial Sequence
  synthesized
aaggcttctccagaactgacttgcacctggccgtggttccagtgctgacagcattaatctcttaccataactacctggacaaaaccaaacaggtaggagg

SEQ ID NO: 77 - DNA, 100 bp, Artificial Sequence
  synthesized
gcattaatctcttaccataactacctggacaaaaccaaacaggtaggaggtcagagcaggacaggcgagcttgatggggcctgggattcgagggcctggc

SEQ ID NO: 78 - DNA, 100 bp, Artificial Sequence
  synthesized
gcgaggctgcctctgctgcaagcgggtggggcctgaggtgtcctgtctcctgcagcgcgagatggtctactgcctggagcagggcctcatccaccgctgt

SEQ ID NO: 79 - DNA, 100 bp, Artificial Sequence
  synthesized
tgcagcgcgagatggtctactgcctggagcagggcctcatccaccgctgtgccagccagtgcgtcgtggccttgtccatctgcagcgtggagatgcctga

SEQ ID NO: 80 - DNA, 100 bp, Artificial Sequence
  synthesized
gccagccagtgcgtcgtggccttgtccatctgcagcgtggagatgcctgacatcatcatcaaggcgctgcctgttctggtggtgaagctcacgcacatct

SEQ ID NO: 81 - DNA, 100 bp, Artificial Sequence
  synthesized
catcatcatcaaggcgctgcctgttctggtggtgaagctcacgcacatctcagccacagccagcatggccgtcccactgctggagttcctgtccagtgag

SEQ ID NO: 82 - DNA, 100 bp, Artificial Sequence
  synthesized
cagccacagccagcatggccgtcccactgctggagttcctgtccagtgagtccccgccctgcctgcgcatgcacccgagaggttcgggctgtgtaacctg

SEQ ID NO: 83 - DNA, 100 bp, Artificial Sequence
  synthesized
agaggcgctgcacgggaccccggctcccctgaccaccctctccattaccgcagctctggccaggctgccgcacctctacaggaactttgccgcggagcag

SEQ ID NO: 84 - DNA, 100 bp, Artificial Sequence
  synthesized
cagctctggccaggctgccgcacctctacaggaactttgccgcggagcagtatgccagtgtgttcgccatctccctgccgtacaccaacccctccaagtg

SEQ ID NO: 85 - DNA, 100 bp, Artificial Sequence
  synthesized
tatgccagtgtgttcgccatctccctgccgtacaccaacccctccaagtgagtggtcgccccaggccctgtgcctcccagccgtggcccccgctaggcct

SEQ ID NO: 86 - DNA, 100 bp, Artificial Sequence
  synthesized
gttttttgcacttcatgccctggggatgtttccctgctgccaggatggagtgccagcccccttctcatctcaggtttaatcagtacatcgtgtgtctggc

SEQ ID NO: 87 - DNA, 100 bp, Artificial Sequence
  synthesized
tgccagcccccttctcatctcaggtttaatcagtacatcgtgtgtctggcccatcacgtcatagccatgtggttcatcaggtgccgcctgcccttccgga

SEQ ID NO: 88 - DNA, 100 bp, Artificial Sequence
  synthesized
ccatcacgtcatagccatgtggttcatcaggtgccgcctgcccttccggaaggattttgtccctttcatcactaaggtgggctcagggccggtgaaggct

SEQ ID NO: 89 - DNA, 100 bp, Artificial Sequence
  synthesized
aggattttgtccctttcatcactaaggtgggctcagggccggtgaaggctgtgtctctcggtaggccagggcttgctttgcccttggctgtccatggtcg

SEQ ID NO: 90 - DNA, 100 bp, Artificial Sequence
  synthesized
cctccagcccccattgccacccctcactgtctgggtgtgctcactctgccagggcctgcggtccaatgtcctcttgtcttttgatgacacccccgagaag

SEQ ID NO: 91 - DNA, 100 bp, Artificial Sequence
  synthesized
agggcctgcggtccaatgtcctcttgtcttttgatgacacccccgagaaggacagcttcagggcccggagtactagtctcaacgagagacccaagaggta

SEQ ID NO: 92 - DNA, 100 bp, Artificial Sequence
  synthesized
gacagcttcagggcccggagtactagtctcaacgagagacccaagaggtacggcctgcgggggtgtgcctggagtcggtgtggggtggggaaggacatgg

SEQ ID NO: 93 - DNA, 100 bp, Artificial Sequence
  synthesized
ttctccccttcccgggagctgggctctctggggcgttggggctccttcctcacccgatagtctgaggatagccagaccccccaaacaaggcttgaataac

SEQ ID NO: 94 - DNA, 100 bp, Artificial Sequence
  synthesized
cacccgatagtctgaggatagccagaccccccaaacaaggcttgaataactctccacccgtgaaagaattcaaggagagctctgcagccgaggccttccg

SEQ ID NO: 95 - DNA, 100 bp, Artificial Sequence
  synthesized
tctccacccgtgaaagaattcaaggagagctctgcagccgaggccttccggtgccgcagcatcagtgtgtctgaacatgtggtccgcaggtagcgggact

SEQ ID NO: 96 - DNA, 100 bp, Artificial Sequence
  synthesized
gtgccgcagcatcagtgtgtctgaacatgtggtccgcaggtagcgggactgtcgggtggggggcacggaccctggagcttggccccgtgagcacctgggt

SEQ ID NO: 97 - DNA, 100 bp, Artificial Sequence
  synthesized
cttggtgataggtggctcggcccgccctacctggcaccctgaccctggtcacggcctctccctccagcaggatacagacgtccctcaccagtgccagctt

SEQ ID NO: 98 - DNA, 100 bp, Artificial Sequence
  synthesized
acggcctctccctccagcaggatacagacgtccctcaccagtgccagcttggggtctgcagatgagaactccgtggcccaggctgacgatagcctgaaaa

SEQ ID NO: 99 - DNA, 100 bp, Artificial Sequence
  synthesized
ggggtctgcagatgagaactccgtggcccaggctgacgatagcctgaaaaacctccacctggagctcacggaaacctgtctggacatgatggctcgatac

SEQ ID NO: 100 - DNA, 100 bp, Artificial Sequence
  synthesized
acctccacctggagctcacggaaacctgtctggacatgatggctcgatacgtcttctccaacttcacggctgtcccgaagaggtccaggcggcactacag

SEQ ID NO: 101 - DNA, 100 bp, Artificial Sequence
  synthesized
gtcttctccaacttcacggctgtcccgaagaggtccaggcggcactacagggctgggcgggcctgcgggagctccacgggcaagctgggtttcacgctcc

SEQ ID NO: 102 - DNA, 100 bp, Artificial Sequence
  synthesized
gcggcactacagggctgggcgggcctgcgggagctccacgggcaagctgggtttcacgctccctgtcttctaggtctcctgtgggcgagttcctcctagc

SEQ ID NO: 103 - DNA, 100 bp, Artificial Sequence
  synthesized
gtttcacgctccctgtcttctaggtctcctgtgggcgagttcctcctagcgggtggcaggaccaaaacctggctggttgggaacaagcttgtcactgtga

SEQ ID NO: 104 - DNA, 100 bp, Artificial Sequence
  synthesized
gggtggcaggaccaaaacctggctggttgggaacaagcttgtcactgtgacgacaagcgtgggaaccgggacccggtcgttactaggcctggactcgggg

SEQ ID NO: 105 - DNA, 100 bp, Artificial Sequence
  synthesized
cgacaagcgtgggaaccgggacccggtcgttactaggcctggactcgggggagctgcagtccggcccggagtcgaggtgactgcaccttcctttcctccg

SEQ ID NO: 106 - DNA, 100 bp, Artificial Sequence
  synthesized
gagctgcagtccggcccggagtcgaggtgactgcaccttcctttcctccgcgcctgccagcctcgacaccggctgtcccgagcccaggcccacgtggcac

SEQ ID NO: 107 - DNA, 100 bp, Artificial Sequence
  synthesized
ggcccacgtggcaccctcgtaccagcctggggactaagtccaccctgtgcgtgggattctcttctcagctccagccccggggtgcatgtgagacagacca

SEQ ID NO: 108 - DNA, 100 bp, Artificial Sequence
  synthesized
gtgggattctcttctcagctccagccccggggtgcatgtgagacagaccaaggaggcgccggccaagctggagtcccaggctgggcagcaggtgtcccgt

SEQ ID NO: 109 - DNA, 100 bp, Artificial Sequence
  synthesized
aggaggcgccggccaagctggagtcccaggctgggcagcaggtgtcccgtggggcccgggatcgggtccgttccatgtcgggtgagccttggccccagcc

SEQ ID NO: 110 - DNA, 100 bp, Artificial Sequence
  synthesized
ggggcccgggatcgggtccgttccatgtcgggtgagccttggccccagccacctccacacaggcaccggggctccctcagttgctgctggtcccagtgtt

SEQ ID NO: 111 - DNA, 100 bp, Artificial Sequence
  synthesized
ttcagcttgaggctggtggttttgcatcaggtaagtggtggtcaccagtcctctgccctcttcttcagggggccatggtcttcgagttggcgccctggac

SEQ ID NO: 112 - DNA, 100 bp, Artificial Sequence
  synthesized
ctctgccctcttcttcagggggccatggtcttcgagttggcgccctggacgtgccggcctcccagttcctgggcagtgccacttctccaggaccacggac

SEQ ID NO: 113 - DNA, 100 bp, Artificial Sequence
  synthesized
gtgccggcctcccagttcctgggcagtgccacttctccaggaccacggactgcaccagccgcgaaacctgagaaggcctcagctggcacccgggttcctg

SEQ ID NO: 114 - DNA, 100 bp, Artificial Sequence
  synthesized
tgcaccagccgcgaaacctgagaaggcctcagctggcacccgggttcctgtgcaggagaagacgaacctggcggcctatgtgcccctgctgacccagggc

SEQ ID NO: 115 - DNA, 100 bp, Artificial Sequence
  synthesized
tgcaggagaagacgaacctggcggcctatgtgcccctgctgacccagggctgggcggagatcctggtccggaggcccacaggtactgggcggggctggcc

SEQ ID NO: 116 - DNA, 100 bp, Artificial Sequence
  synthesized
tgggcggagatcctggtccggaggcccacaggtactgggcggggctggcctgagcgccatctttctgccagtcacccacagagctgtggacactcagggg

SEQ ID NO: 117 - DNA, 100 bp, Artificial Sequence
  synthesized
aggcccctggggggccagagatgggtaaggggaggtactggcctcaggccaaaggtgctgccgcctccgcagggaacaccagctggctgatgagcctgga

SEQ ID NO: 118 - DNA, 100 bp, Artificial Sequence
  synthesized
aaaggtgctgccgcctccgcagggaacaccagctggctgatgagcctggagaacccgctcagccctttctcctcggacatcaacaacatgcccctgcagg

SEQ ID NO: 119 - DNA, 100 bp, Artificial Sequence
  synthesized
gaacccgctcagccctttctcctcggacatcaacaacatgcccctgcaggagctgtctaacgccctcatggcggctgagcgcttcaaggagcaccgggac

SEQ ID NO: 120 - DNA, 100 bp, Artificial Sequence
  synthesized
agctgtctaacgccctcatggcggctgagcgcttcaaggagcaccgggacacagccctgtacaagtcactgtcggtgccggcagccagcacggccaaacc

SEQ ID NO: 121 - DNA, 100 bp, Artificial Sequence
  synthesized
acagccctgtacaagtcactgtcggtgccggcagccagcacggccaaaccccctcctctgcctcgctccaacacaggtgagtggcatggcgggccttggc

SEQ ID NO: 122 - DNA, 100 bp, Artificial Sequence
  synthesized
ccctcctctgcctcgctccaacacaggtgagtggcatggcgggccttggcacgggctctgctcccactggcctggtgctcccggtgacggcaatgtggct

SEQ ID NO: 123 - DNA, 100 bp, Artificial Sequence
  synthesized
ctctgctcgacctgtgtgtagcccctcctcctgctgacgtggccgcacacggccttcccttgcagtggcctctttctcctccctgtaccagtccagctgc

SEQ ID NO: 124 - DNA, 100 bp, Artificial Sequence
  synthesized
ggccttcccttgcagtggcctctttctcctccctgtaccagtccagctgccaaggacagctgcacaggagcgtttcctgggcaggtatcgcctctcagag

SEQ ID NO: 125 - DNA, 100 bp, Artificial Sequence
  synthesized
caaggacagctgcacaggagcgtttcctgggcaggtatcgcctctcagagggaagcggttggctgcagagcgccactctgcctcataggtgctgtgctcg

SEQ ID NO: 126 - DNA, 100 bp, Artificial Sequence
  synthesized
ggccacgtcagggccagggcctggcccagccccacatccagcagccccgtctgtgtcctcccagactccgccgtggtcatggaggagggaagtccgggcg

SEQ ID NO: 127 - DNA, 100 bp, Artificial Sequence
  synthesized
ctgtgtcctcccagactccgccgtggtcatggaggagggaagtccgggcgaggttcctgtgctggtggagcccccagggttggaggacgttgaggcagcg

SEQ ID NO: 128 - DNA, 100 bp, Artificial Sequence
  synthesized
aggttcctgtgctggtggagcccccagggttggaggacgttgaggcagcgctaggcatggacaggcgcacggatgcctacagcagggtgagtgtggctca

SEQ ID NO: 129 - DNA, 100 bp, Artificial Sequence
  synthesized
ctaggcatggacaggcgcacggatgcctacagcagggtgagtgtggctcagagcctggaccctgctgacctcggggggctccttaggggaggcagggctc

SEQ ID NO: 130 - DNA, 100 bp, Artificial Sequence
  synthesized
ggtgggctcgagggtgcctgctgacaggggttctctttgggatggtcctttctagtcgtcctcagtctccagccaggaggagaagtcgctccacgcggag

SEQ ID NO: 131 - DNA, 100 bp, Artificial Sequence
  synthesized
tctagtcgtcctcagtctccagccaggaggagaagtcgctccacgcggaggagctggttggcaggggcatccccatcgagcgagtcgtctcctcggaggg

SEQ ID NO: 132 - DNA, 100 bp, Artificial Sequence
  synthesized
gagctggttggcaggggcatccccatcgagcgagtcgtctcctcggagggtggccggccctctgtggacctctccttccagccctcgcagcccctgagca

SEQ ID NO: 133 - DNA, 100 bp, Artificial Sequence
  synthesized
tggccggccctctgtggacctctccttccagccctcgcagcccctgagcaagtccagctcctctcccgagctgcagactctgcaggacatcctcggggac

SEQ ID NO: 134 - DNA, 100 bp, Artificial Sequence
  synthesized
agtccagctcctctcccgagctgcagactctgcaggacatcctcggggaccctggggacaaggccgacgtgggccggctgagccctgaggttaaggcccg

SEQ ID NO: 135 - DNA, 100 bp, Artificial Sequence
  synthesized
cctggggacaaggccgacgtgggccggctgagccctgaggttaaggcccggtcacagtcagggaccctggacggggaaagtgctgcctggtcggcctcgg

SEQ ID NO: 136 - DNA, 100 bp, Artificial Sequence
  synthesized
gtcacagtcagggaccctggacggggaaagtgctgcctggtcggcctcgggcgaagacagtcggggccagcccgagggtcccttgccttccagctccccc

SEQ ID NO: 137 - DNA, 100 bp, Artificial Sequence
  synthesized
gcgaagacagtcggggccagcccgagggtcccttgccttccagctccccccgctcgcccagtggcctccggccccgaggttacaccatctccgactcggc

SEQ ID NO: 138 - DNA, 100 bp, Artificial Sequence
  synthesized
cgctcgcccagtggcctccggccccgaggttacaccatctccgactcggccccatcacgcaggggcaagagagtagagagggacgccttaaagagcagag

SEQ ID NO: 139 - DNA, 100 bp, Artificial Sequence
  synthesized
cccatcacgcaggggcaagagagtagagagggacgccttaaagagcagagccacagcctccaatgcagagaaagtgccaggcatcaaccccaggtgggcc

SEQ ID NO: 140 - DNA, 100 bp, Artificial Sequence
  synthesized
ccacagcctccaatgcagagaaagtgccaggcatcaaccccaggtgggcctcttgcttccgggcggggctcctgacacctctcctgcgggaacctggtgc

SEQ ID NO: 141 - DNA, 100 bp, Artificial Sequence
  synthesized
tgggctgtggctgccctggccaggccctcacctgggtgcccaccatcccctccctgtgcagtttcgtgttcctgcagctctaccattcccccttctttgg

SEQ ID NO: 142 - DNA, 100 bp, Artificial Sequence
  synthesized
tccctgtgcagtttcgtgttcctgcagctctaccattcccccttctttggcgacgagtcaaacaagccaatcctgctgcccaatgaggtaggcgtggcct

SEQ ID NO: 143 - DNA, 100 bp, Artificial Sequence
  synthesized
cgacgagtcaaacaagccaatcctgctgcccaatgaggtaggcgtggcctccctctcctgcatccgctggagctgtgtggctcgggtgaatggtgggggg

SEQ ID NO: 144 - DNA, 100 bp, Artificial Sequence
  synthesized
cggcctcctgtggacgggcgtctggggctcaggcagggctctgtgtgccacagtcacagtcctttgagcggtcggtgcagctcctcgaccagatcccatc

SEQ ID NO: 145 - DNA, 100 bp, Artificial Sequence
  synthesized
cagtcacagtcctttgagcggtcggtgcagctcctcgaccagatcccatcatacgacacccacaagatcgccgtcctgtatgttggagaaggccaggtga

SEQ ID NO: 146 - DNA, 100 bp, Artificial Sequence
  synthesized
atacgacacccacaagatcgccgtcctgtatgttggagaaggccaggtgaggctgcggggccggcctaggtgcctggacagggccagctgggcctcagcc

SEQ ID NO: 147 - DNA, 100 bp, Artificial Sequence
  synthesized
cggggcagggcccggcccgggagtgatgccaccctgcctctcccctctccccacagagcaacagcgagctcgccatcctgtccaatgagcatggctccta

SEQ ID NO: 148 - DNA, 100 bp, Artificial Sequence
  synthesized
ccacagagcaacagcgagctcgccatcctgtccaatgagcatggctcctacaggtacacggagttcctgacgggcctgggccggctcatcgagctgaagg

SEQ ID NO: 149 - DNA, 100 bp, Artificial Sequence
  synthesized
caggtacacggagttcctgacgggcctgggccggctcatcgagctgaaggactgccagccggacaaggtgtacctgggaggcctggacgtgtgtggtgag

SEQ ID NO: 150 - DNA, 100 bp, Artificial Sequence
  synthesized
actgccagccggacaaggtgtacctgggaggcctggacgtgtgtggtgaggacggccagttcacctactgctggcacgatgacatcatgcaaggtacggc

SEQ ID NO: 151 - DNA, 100 bp, Artificial Sequence
  synthesized
gacggccagttcacctactgctggcacgatgacatcatgcaaggtacggcctggcgcctacccgctcctgctgccccaggcctcagggcacggctcccat

SEQ ID NO: 152 - DNA, 100 bp, Artificial Sequence
  synthesized
acagagggcctcagcactggccccacaaacccatccggccctgctcaccctcagccgtcttccacatcgccaccctgatgcccaccaaggacgtggacaa

SEQ ID NO: 153 - DNA, 100 bp, Artificial Sequence
  synthesized
tcagccgtcttccacatcgccaccctgatgcccaccaaggacgtggacaagcaccgctgcgacaagaagcgccacctgggcaacgactttgtgtccattg

SEQ ID NO: 154 - DNA, 100 bp, Artificial Sequence
  synthesized
gcaccgctgcgacaagaagcgccacctgggcaacgactttgtgtccattgtctacaatgactccggtgaggacttcaagcttggcaccatcaaggtgagt

SEQ ID NO: 155 - DNA, 100 bp, Artificial Sequence
  synthesized
tctacaatgactccggtgaggacttcaagcttggcaccatcaaggtgagtgaggggccgtcagtgaggctgggccccaggcaggtgcccactgctgtgtc

SEQ ID NO: 156 - DNA, 100 bp, Artificial Sequence
  synthesized
ccgagatcagccttcagcacacgctgtgtgcggggatgaccctttctcttgtccgggcagggccagttcaactttgtccacgtgatcgtcaccccgctgg

SEQ ID NO: 157 - DNA, 100 bp, Artificial Sequence
  synthesized
gtccgggcagggccagttcaactttgtccacgtgatcgtcaccccgctggactacgagtgcaacctggtgtccctgcagtgcaggaaaggtagggccggg

SEQ ID NO: 158 - DNA, 100 bp, Artificial Sequence
  synthesized
actacgagtgcaacctggtgtccctgcagtgcaggaaaggtagggccgggtggggccctgcagtgcaggaaaggtagggccgggtggggccctgcagtgt

SEQ ID NO: 159 - DNA, 100 bp, Artificial Sequence
  synthesized
tgcagtgtggcgccaagagccctgggcctggcgtgaccaccaagtctccccagacatggagggccttgtggacaccagcgtggccaagatcgtgtctgac

SEQ ID NO: 160 - DNA, 100 bp, Artificial Sequence
  synthesized
cagacatggagggccttgtggacaccagcgtggccaagatcgtgtctgaccgcaacctgcccttcgtggcccgccagatggccctgcacgcaaatgtgag

SEQ ID NO: 161 - DNA, 100 bp, Artificial Sequence
  synthesized
cgcaacctgcccttcgtggcccgccagatggccctgcacgcaaatgtgagtgggggtgggtccaggcgtgagctggtgggacaggcccaggtgccacctg

SEQ ID NO: 162 - DNA, 100 bp, Artificial Sequence
  synthesized
aggcccaggtgccacctgatagtgagctcaccccctgcctacgtccccagatggcctcacaggtgcatcatagccgctccaaccccaccgatatctaccc

SEQ ID NO: 163 - DNA, 100 bp, Artificial Sequence
  synthesized
atggcctcacaggtgcatcatagccgctccaaccccaccgatatctacccctccaagtggattgcccggctccgccacatcaagcggctccgccagcggg

SEQ ID NO: 164 - DNA, 100 bp, Artificial Sequence
  synthesized
ctccaagtggattgcccggctccgccacatcaagcggctccgccagcgggtagggaatatggggctccctcagcggggtgtgctggctgcccaagctgtg

SEQ ID NO: 165 - DNA, 100 bp, Artificial Sequence
  synthesized
gcgggtgtgtgggcagagcggttgccacgcctcccagacttactgcccaagccgcctctgccttcagatctgcgaggaagccgcctactccaaccccagc

SEQ ID NO: 166 - DNA, 100 bp, Artificial Sequence
  synthesized
gccgcctctgccttcagatctgcgaggaagccgcctactccaaccccagcctacctctggtgcaccctccgtcccatagcaaagcccctgcacagactcc

SEQ ID NO: 167 - DNA, 100 bp, Artificial Sequence
  synthesized
ctacctctggtgcaccctccgtcccatagcaaagcccctgcacagactccagccgagcccacacctggctatgaggtgggccagcggaagcgcctcatct

SEQ ID NO: 168 - DNA, 100 bp, Artificial Sequence
  synthesized
agccgagcccacacctggctatgaggtgggccagcggaagcgcctcatctcctcggtggaggacttcaccgagtttgtgtgaggccggggccctccctcc

SEQ ID NO: 169 - DNA, 100 bp, Artificial Sequence
  synthesized
cctcggtggaggacttcaccgagtttgtgtgaggccggggccctccctcctgcactggccttggacggtattgcctgtcagtgaaataaataaagtcctg

SEQ ID NO: 170 - DNA, 100 bp, Artificial Sequence
  synthesized
gtgcattcacacctcctgttctgtgccaacaatatgcaagttaacactgattgaccatcattccttagctgtgttcatgatgagtctcattgtagtccat

SEQ ID NO: 171 - DNA, 100 bp, Artificial Sequence
  synthesized
ttgaccatcattccttagctgtgttcatgatgagtctcattgtagtccatgatatgtagctgtccaacactgtccggggtcgggggagacgggtgagggc

SEQ ID NO: 172 - DNA, 100 bp, Artificial Sequence
  synthesized
gatatgtagctgtccaacactgtccggggtcgggggagacgggtgagggccatctaggttcaggggaatcttggcttccacacccaagtctttgcccagt

SEQ ID NO: 173 - DNA, 100 bp, Artificial Sequence
  synthesized
catctaggttcaggggaatcttggcttccacacccaagtctttgcccagttctgtctttaggctctcagaaaggctactggtcatgccgtcctcatcaca

SEQ ID NO: 174 - DNA, 100 bp, Artificial Sequence
  synthesized
tctgtctttaggctctcagaaaggctactggtcatgccgtcctcatcacactggctctcgctcttattacgaaataactctcgagccttcatacccagga

SEQ ID NO: 175 - DNA, 100 bp, Artificial Sequence
  synthesized
ctggctctcgctcttattacgaaataactctcgagccttcatacccaggaagctttttgaactgggaagtgagcccacagtggtggggatgctggcagac

SEQ ID NO: 176 - DNA, 100 bp, Artificial Sequence
  synthesized
agctttttgaactgggaagtgagcccacagtggtggggatgctggcagacgcttctcccatagtcgtctcccaccgactgctgaatgggcctgccctctg

SEQ ID NO: 177 - DNA, 100 bp, Artificial Sequence
  synthesized
gcttctcccatagtcgtctcccaccgactgctgaatgggcctgccctctggtgtgggggtttctctggggtagaaagctcgctgctgctgctgctgctgc

SEQ ID NO: 178 - DNA, 100 bp, Artificial Sequence
  synthesized
gtgtgggggtttctctggggtagaaagctcgctgctgctgctgctgctgcctccaccacctctgcttccactactgccccgggcgctgctgggcctgggg

SEQ ID NO: 179 - DNA, 100 bp, Artificial Sequence
  synthesized
ctccaccacctctgcttccactactgccccgggcgctgctgggcctgggggtcttggtctcaccgttgtggccagatgcctcttcattgtgccctaccat

SEQ ID NO: 180 - DNA, 100 bp, Artificial Sequence
  synthesized
gtcttggtctcaccgttgtggccagatgcctcttcattgtgccctaccatggaatctgagcacccgtcattacaacagtcaagcctgtaagaaagccggg

SEQ ID NO: 181 - DNA, 100 bp, Artificial Sequence
  synthesized
ggaatctgagcacccgtcattacaacagtcaagcctgtaagaaagccggggaggaaaaaaggagctggtgattggactgtccacattcggaggatgtgga

SEQ ID NO: 182 - DNA, 100 bp, Artificial Sequence
  synthesized
cgtgacacagtccttatgctggaattggcagcttagtcccaaggtcatgaatcagttctttgttcctacctttcttctgctgcttcagctgcttctgctt

SEQ ID NO: 183 - DNA, 100 bp, Artificial Sequence
  synthesized
atcagttctttgttcctacctttcttctgctgcttcagctgcttctgctttttcttcttcaagttttttcaggaggccatctttctccaacctgccatat

SEQ ID NO: 184 - DNA, 100 bp, Artificial Sequence
  synthesized
tttcttcttcaagttttttcaggaggccatctttctccaacctgccatataaatctaagatctccaattcaaacacctgggttatccttttctgagcctc

SEQ ID NO: 185 - DNA, 100 bp, Artificial Sequence
  synthesized
aaatctaagatctccaattcaaacacctgggttatccttttctgagcctcatacctgctctctgcggcctgcagctgtcctctgaaagatacagaccagc

SEQ ID NO: 186 - DNA, 100 bp, Artificial Sequence
  synthesized
atacctgctctctgcggcctgcagctgtcctctgaaagatacagaccagccagaatataggaagttccacttaataaaaacacaaaagcctttcctgatg

SEQ ID NO: 187 - DNA, 100 bp, Artificial Sequence
  synthesized
aatataggaagttccacttaataaaaacacaaaagcctttcctgatgaaagttaccttgcctggagtttgacatcctctagatatttcttctgttccaaa

SEQ ID NO: 188 - DNA, 100 bp, Artificial Sequence
  synthesized
gttaccttgcctggagtttgacatcctctagatatttcttctgttccaaaagaaggtggtctttcttggccaggtgagattccagttccaaaatccgttt

SEQ ID NO: 189 - DNA, 100 bp, Artificial Sequence
  synthesized
agaaggtggtctttcttggccaggtgagattccagttccaaaatccgtttttgggaggtatcaagcctctgagtctgctggagaacatggcttctgtttt

SEQ ID NO: 190 - DNA, 100 bp, Artificial Sequence
  synthesized
ttgggaggtatcaagcctctgagtctgctggagaacatggcttctgtttttttctagctctttccgataggcggctttcatcatttctacttcctgaaaa

SEQ ID NO: 191 - DNA, 100 bp, Artificial Sequence
  synthesized
tttctagctctttccgataggcggctttcatcatttctacttcctgaaaaaaaaaaaaaaaaaagactggaattagtacttataaaaaataaacatgctg

SEQ ID NO: 192 - DNA, 100 bp, Artificial Sequence
  synthesized
gacatactgtctgggtctgaaacgctttccccactaaggtctggctcccgagccctggcatacctttgtggtatctgagtgcttgttctgcagttgttcc

SEQ ID NO: 193 - DNA, 100 bp, Artificial Sequence
  synthesized
agccctggcatacctttgtggtatctgagtgcttgttctgcagttgttccaaatagagctcgttgacctccccaagaaccaacagctgcctgttcaagaa

SEQ ID NO: 194 - DNA, 100 bp, Artificial Sequence
  synthesized
aaatagagctcgttgacctccccaagaaccaacagctgcctgttcaagaactccatctgctgctggaccgactcactgtttgagagctaaccaaaaaaca

SEQ ID NO: 195 - DNA, 100 bp, Artificial Sequence
  synthesized
ctccatctgctgctggaccgactcactgtttgagagctaaccaaaaaacatgagcaaagtgaaaaatccgacgacataaaactagcacatagacgtcatt

SEQ ID NO: 196 - DNA, 100 bp, Artificial Sequence
  synthesized
acctgtctgaaggaagaatgttagcaaatggtgtttcagcagattcaggtctgcctcatttcttcttaccttttgggaaacctgactgagcagcagctca

SEQ ID NO: 197 - DNA, 100 bp, Artificial Sequence
  synthesized
ctgcctcatttcttcttaccttttgggaaacctgactgagcagcagctcagtgtgacacaccttgttgttggccttcttcagttctatccgcagctccgc

SEQ ID NO: 198 - DNA, 100 bp, Artificial Sequence
  synthesized
gtgtgacacaccttgttgttggccttcttcagttctatccgcagctccgcaatcatgttcctgcagtcctccagcttcgtctgcccaaagagacgtggac

SEQ ID NO: 199 - DNA, 100 bp, Artificial Sequence
  synthesized
aatcatgttcctgcagtcctccagcttcgtctgcccaaagagacgtggacatgaagtttgaggaacaccaacaggccagatcacaggcctacctagccac

SEQ ID NO: 200 - DNA, 100 bp, Artificial Sequence
  synthesized
ctccccactgctctccggcattctcgcagttggctttgcctggtgctgcagtttatacctgtaattcctggctctggttgtagaattcctctcggtcatg

SEQ ID NO: 201 - DNA, 100 bp, Artificial Sequence
  synthesized
gtttatacctgtaattcctggctctggttgtagaattcctctcggtcatgctgcagctgtctgatctggctgtggagcttggttaccatagtgtcacgct

SEQ ID NO: 202 - DNA, 100 bp, Artificial Sequence
  synthesized
ctgcagctgtctgatctggctgtggagcttggttaccatagtgtcacgctgctcctggagctgattgtatctagcttgttctttctgcagactaaccttc

SEQ ID NO: 203 - DNA, 100 bp, Artificial Sequence
  synthesized
gctcctggagctgattgtatctagcttgttctttctgcagactaaccttccacatctggatgtccttctcttgtaacttcaactgatctttctagcagag

SEQ ID NO: 204 - DNA, 100 bp, Artificial Sequence
  synthesized
cacatctggatgtccttctcttgtaacttcaactgatctttctagcagagaccagaaatgtcatcattttagctgtcttccaacacaggcaatttaacac

SEQ ID NO: 205 - DNA, 100 bp, Artificial Sequence
  synthesized
aagctatcatgctgacccaaaacaaaacaaaaagcaagctccacctgtcccctccccagtcctcaccatggcagcattatgttcctccagagctgctgct

SEQ ID NO: 206 - DNA, 100 bp, Artificial Sequence
  synthesized
cctccccagtcctcaccatggcagcattatgttcctccagagctgctgctttgatcaccttgcggaggagccgcctgttccggagggcatgctgctgcct

SEQ ID NO: 207 - DNA, 100 bp, Artificial Sequence
  synthesized
ttgatcaccttgcggaggagccgcctgttccggagggcatgctgctgcctcttaaaacgctcatagagtaactggttgtgcagtaaaagcaactggtctc

SEQ ID NO: 208 - DNA, 100 bp, Artificial Sequence
  synthesized
cttaaaacgctcatagagtaactggttgtgcagtaaaagcaactggtctcggagggtgcggatctcatctgaaggaggagagcctgattgtaaagcagag

SEQ ID NO: 209 - DNA, 100 bp, Artificial Sequence
  synthesized
ggagggtgcggatctcatctgaaggaggagagcctgattgtaaagcagagggagggtggcagaaatgccttttacagatggttcaatcaagcccccttcc

SEQ ID NO: 210 - DNA, 100 bp, Artificial Sequence
  synthesized
aagcaagcaggaaccatgtgggctggatttggagctaaagtaacaactttacctccaaagtgggtccagtcgacagacttgctgggtaaaggcaacctag

SEQ ID NO: 211 - DNA, 100 bp, Artificial Sequence
  synthesized
acctccaaagtgggtccagtcgacagacttgctgggtaaaggcaacctaggaagaaagtttttgagtaacaaagttaccgatcttaccaagaaaaaaacg

SEQ ID NO: 212 - DNA, 100 bp, Artificial Sequence
  synthesized
ctcttacactttctgtacttcacaataaaatggaccatttaacacagaagagagtgccccagtcccttacttgttcagctccttgctgtgcgcgtctgct

SEQ ID NO: 213 - DNA, 100 bp, Artificial Sequence
  synthesized
agagtgccccagtcccttacttgttcagctccttgctgtgcgcgtctgctccctgctgtatcagtctgtccagcacttccattggggaggtagagggcac

SEQ ID NO: 214 - DNA, 100 bp, Artificial Sequence
  synthesized
ccctgctgtatcagtctgtccagcacttccattggggaggtagagggcacaccatcttcctctgtgtttccttttgctttctttaacagctcctcagtct

SEQ ID NO: 215 - DNA, 100 bp, Artificial Sequence
  synthesized
accatcttcctctgtgtttccttttgctttctttaacagctcctcagtcttcctgatgacaaaatgatgggctgtctttggcaatgccacctcaaaaaga

SEQ ID NO: 216 - DNA, 100 bp, Artificial Sequence
  synthesized
tcctgatgacaaaatgatgggctgtctttggcaatgccacctcaaaaagatgatcatacgggggaggctgcccgcttccaaagcccactctcgtcggagg

SEQ ID NO: 217 - DNA, 100 bp, Artificial Sequence
  synthesized
tgatcatacgggggaggctgcccgcttccaaagcccactctcgtcggaggtggaattttacaaggactgggagtgaagatactggtctccaaagaagtct

SEQ ID NO: 218 - DNA, 100 bp, Artificial Sequence
  synthesized
tggaattttacaaggactgggagtgaagatactggtctccaaagaagtctggcattccctgtctcccgcagggctttcatcagcactgccgcagggcagg

SEQ ID NO: 219 - DNA, 100 bp, Artificial Sequence
  synthesized
ggcattccctgtctcccgcagggctttcatcagcactgccgcagggcaggtctatgggagtaaaggcttgctttggtgtgtcaggcccaagcttgtccag

SEQ ID NO: 220 - DNA, 100 bp, Artificial Sequence
  synthesized
tctatgggagtaaaggcttgctttggtgtgtcaggcccaagcttgtccagggaggagtgtaaaggctcagggttcacgctggcgccctgagaactggagg

SEQ ID NO: 221 - DNA, 100 bp, Artificial Sequence
  synthesized
ggaggagtgtaaaggctcagggttcacgctggcgccctgagaactggaggctgccgagtgggtcttccgctgagaacctgggagactgtctcggtaaaag

SEQ ID NO: 222 - DNA, 100 bp, Artificial Sequence
  synthesized
ctgccgagtgggtcttccgctgagaacctgggagactgtctcggtaaaagggagagtcaaagcctcctcgaggaaccacaggctctgcctctgctgtggt

SEQ ID NO: 223 - DNA, 100 bp, Artificial Sequence
  synthesized
ggagagtcaaagcctcctcgaggaaccacaggctctgcctctgctgtggtgatctcagaaagttctctagatattgcagctgagaggaagagaggaaaca

SEQ ID NO: 224 - DNA, 100 bp, Artificial Sequence
  synthesized
gatctcagaaagttctctagatattgcagctgagaggaagagaggaaacaaaagaaatggcagtcggtattccacctgggaaagactaggcagtttgggt

SEQ ID NO: 225 - DNA, 100 bp, Artificial Sequence
  synthesized
gcacaaaatcccagatttatagcagagcgagggtcaggttttatcaactcatagcaatcccacatacattaccttcttctttatctttttcaatactatc

SEQ ID NO: 226 - DNA, 100 bp, Artificial Sequence
  synthesized
atagcaatcccacatacattaccttcttctttatctttttcaatactatcttcttcagaggccagatcacctaaaaaccctggaagatcacttagagtga

SEQ ID NO: 227 - DNA, 100 bp, Artificial Sequence
  synthesized
ttcttcagaggccagatcacctaaaaaccctggaagatcacttagagtgacagaacctttgctgccaggtggctcttctgaagagaaacaaagacaactg

SEQ ID NO: 228 - DNA, 100 bp, Artificial Sequence
  synthesized
cagaacctttgctgccaggtggctcttctgaagagaaacaaagacaactgaagtcaaagaaatacagtgtaatccctgtaagtgtaaaactgcttacact

SEQ ID NO: 229 - DNA, 100 bp, Artificial Sequence
  synthesized
ttcttaaacacatataacccaattagaagaggcaagcaaggcctgtagtaacgcagaaattttacctgatcctctgtcattcagaagatggtgttgtctg

SEQ ID NO: 230 - DNA, 100 bp, Artificial Sequence
  synthesized
acgcagaaattttacctgatcctctgtcattcagaagatggtgttgtctgtgtagacatggtcttgcagaatccattctctcttcctgaaaagataagta

SEQ ID NO: 231 - DNA, 100 bp, Artificial Sequence
  synthesized
tgtagacatggtcttgcagaatccattctctcttcctgaaaagataagtatcatttatatcacaagacgaaaaatgttgcacatgttctcgagcatattg

SEQ ID NO: 232 - DNA, 100 bp, Artificial Sequence
  synthesized
atcacaccttgagagcagcttgttagtccattttcaattattctgattcaaacccattgcattttaggtcagaattctatctggcataattaggcttctc

SEQ ID NO: 233 - DNA, 100 bp, Artificial Sequence
  synthesized
aacccattgcattttaggtcagaattctatctggcataattaggcttctcaaagtgaggcttgcaagtgagtcactgtgcctgggcagagggatagcaga

SEQ ID NO: 234 - DNA, 100 bp, Artificial Sequence
  synthesized
aaagtgaggcttgcaagtgagtcactgtgcctgggcagagggatagcagacgagctggatcgcaccttcctggggggtgtgactgtggcctgggggagtg

SEQ ID NO: 235 - DNA, 100 bp, Artificial Sequence
  synthesized
cgagctggatcgcaccttcctggggggtgtgactgtggcctgggggagtgaaatgtgcacgtagtcatccgaatgacagagtggggctggaggaggagag

SEQ ID NO: 236 - DNA, 100 bp, Artificial Sequence
  synthesized
aaatgtgcacgtagtcatccgaatgacagagtggggctggaggaggagaggttgctggggttcccagaggagttccttttccacctgcttagagacaagg

SEQ ID NO: 237 - DNA, 100 bp, Artificial Sequence
  synthesized
gttgctggggttcccagaggagttccttttccacctgcttagagacaagggcagaacatatatgaacactgagcccaactattagaaaaactgccgattt

SEQ ID NO: 238 - DNA, 100 bp, Artificial Sequence
  synthesized
gagagctcctcctgccattaaaggcaggccaaaaccaactaatcaaatccaacctaagacatacataccagttgtaccaaagactttactgtaagggtgt

SEQ ID NO: 239 - DNA, 100 bp, Artificial Sequence
  synthesized
aacctaagacatacataccagttgtaccaaagactttactgtaagggtgtgacagatcaggtgggacatttccaggagaagttggaggagtggtcatacc

SEQ ID NO: 240 - DNA, 100 bp, Artificial Sequence
  synthesized
gacagatcaggtgggacatttccaggagaagttggaggagtggtcataccacaaaccatagatgggctccaaagagtagcctgggaagttaataaagtac

SEQ ID NO: 241 - DNA, 100 bp, Artificial Sequence
  synthesized
acaaaccatagatgggctccaaagagtagcctgggaagttaataaagtacatcagcagtggcaaaggaatgctaagtcatccacgaggtttatatccatg

SEQ ID NO: 242 - DNA, 100 bp, Artificial Sequence
  synthesized
ggatccttaaaagtgactcctgaaatgagcagtgtgaaattttcccaaccacatactaaatctgacccaaagggtcagcttcaccagaaagcagaggaga

SEQ ID NO: 243 - DNA, 100 bp, Artificial Sequence
  synthesized
acatactaaatctgacccaaagggtcagcttcaccagaaagcagaggagagagcaggcacactagttgacaccatacttgtggtggttcagttatcagcc

SEQ ID NO: 244 - DNA, 100 bp, Artificial Sequence
  synthesized
gagcaggcacactagttgacaccatacttgtggtggttcagttatcagccgtgtcgatggggaactcagagtctgaggtagctgccctggcatatttaac

SEQ ID NO: 245 - DNA, 100 bp, Artificial Sequence
  synthesized
gtgtcgatggggaactcagagtctgaggtagctgccctggcatatttaacaacatcagccgagacgtggagtaaggggtagaagtagcacaccctaaaat

SEQ ID NO: 246 - DNA, 100 bp, Artificial Sequence
  synthesized
aacatcagccgagacgtggagtaaggggtagaagtagcacaccctaaaatggaagagaagaacacagggggttagtgtgtggttttaggttattctggtt

SEQ ID NO: 247 - DNA, 100 bp, Artificial Sequence
  synthesized
acaaataatgttttccagagacaaagttgcaaaacagataagtaccaaagacactttttaccatagctattctgtgtgtcagcataagggctggtggtga

SEQ ID NO: 248 - DNA, 100 bp, Artificial Sequence
  synthesized
acactttttaccatagctattctgtgtgtcagcataagggctggtggtgacatcggctgaacgatgaggaaagcgggctgagatttggtgagacacagaa

SEQ ID NO: 249 - DNA, 100 bp, Artificial Sequence
  synthesized
catcggctgaacgatgaggaaagcgggctgagatttggtgagacacagaatagccatcttcatatgaggcttctgtgggatccagagagattttggcaca

SEQ ID NO: 250 - DNA, 100 bp, Artificial Sequence
  synthesized
tagccatcttcatatgaggcttctgtgggatccagagagattttggcacactcgatcacaacatcatgagtttctaatctcttccacctgtaaaatgcaa

SEQ ID NO: 251 - DNA, 100 bp, Artificial Sequence
  synthesized
ctcgatcacaacatcatgagtttctaatctcttccacctgtaaaatgcaatgaaagtcaagaaatgcaaactgtaatcaactgaattaaatacttcagag

SEQ ID NO: 252 - DNA, 100 bp, Artificial Sequence
  synthesized
ctcctagatcacattttcaatctctcgaaagattctttaaaattttgacactagtttctataccttcgagggtccagttcatggtccttggatccagtca

SEQ ID NO: 253 - DNA, 100 bp, Artificial Sequence
  synthesized
ctagtttctataccttcgagggtccagttcatggtccttggatccagtcactaattccggatgaattcgcacatgctccatcattggctagaagagttgg

SEQ ID NO: 254 - DNA, 100 bp, Artificial Sequence
  synthesized
ctaattccggatgaattcgcacatgctccatcattggctagaagagttgggttgacaaattataaagggctgaatgtttgtggaacatccaaatgatgga

SEQ ID NO: 255 - DNA, 100 bp, Artificial Sequence
  synthesized
atacaaaaggtataaatgcagcctatctaaacagtatactaagtagcaaacaaacaagcagtttcaatttaccttgaccacttcttcaaaagtctccagg

SEQ ID NO: 256 - DNA, 100 bp, Artificial Sequence
  synthesized
caaacaagcagtttcaatttaccttgaccacttcttcaaaagtctccaggttttctttcatactgtaatgagaacgcaaaaaggagacgaagttgcaagg

SEQ ID NO: 257 - DNA, 100 bp, Artificial Sequence
  synthesized
ttttctttcatactgtaatgagaacgcaaaaaggagacgaagttgcaagggtacattccataaaggcgatgaaagagtgcgtacacactggcatggagat

SEQ ID NO: 258 - DNA, 100 bp, Artificial Sequence
  synthesized
gtacattccataaaggcgatgaaagagtgcgtacacactggcatggagatggacgagatagacttccgccacgtggcctagaaaaggaacccgttgagaa

SEQ ID NO: 259 - DNA, 100 bp, Artificial Sequence
  synthesized
ggacgagatagacttccgccacgtggcctagaaaaggaacccgttgagaagagcctcttagttggagacagattgaggagtgcaaaacagctataaacaa

SEQ ID NO: 260 - DNA, 100 bp, Artificial Sequence
  synthesized
aatgaaagcattcacctcacagggcccaacaggtatatgaggagatctgtacctggtttcttcaggcaccatgatgacagacggccaaaaatgtcaaaga

SEQ ID NO: 261 - DNA, 100 bp, Artificial Sequence
  synthesized
acctggtttcttcaggcaccatgatgacagacggccaaaaatgtcaaagaaatcaagaagatgctgtttcccagactgtggaatcattggtagcatggtt

SEQ ID NO: 262 - DNA, 100 bp, Artificial Sequence
  synthesized
aatcaagaagatgctgtttcccagactgtggaatcattggtagcatggttatcaacaccaagacgcctgttgtgaggacaacgacgtcagtgtccatctg

SEQ ID NO: 263 - DNA, 100 bp, Artificial Sequence
  synthesized
atcaacaccaagacgcctgttgtgaggacaacgacgtcagtgtccatctgcaggagaaaaggtcaaacaggaaacgtctgtcaggcactggcaccaggat

SEQ ID NO: 264 - DNA, 100 bp, Artificial Sequence
  synthesized
gctttaagttgcctaaaatttcagaaactatactcataaaaccatttcattcaaatccttacaaacatcctaccttgagacattttagtaaagaaggcaa

SEQ ID NO: 265 - DNA, 100 bp, Artificial Sequence
  synthesized
tcaaatccttacaaacatcctaccttgagacattttagtaaagaaggcaaaagaggtgcttgagagagcttatgcttccaagatggctgcagtcttatga

SEQ ID NO: 266 - DNA, 100 bp, Artificial Sequence
  synthesized
aagaggtgcttgagagagcttatgcttccaagatggctgcagtcttatgacatgacccagtaacgagaggatggataaacgagtggcggctttgcccaca

SEQ ID NO: 267 - DNA, 100 bp, Artificial Sequence
  synthesized
catgacccagtaacgagaggatggataaacgagtggcggctttgcccacatattcgttaatcctgtccaagaggtgctgaaaatgtaaaagaacaagggc

SEQ ID NO: 268 - DNA, 100 bp, Artificial Sequence
  synthesized
tattcgttaatcctgtccaagaggtgctgaaaatgtaaaagaacaagggcagtcctcacatgaatgtatgaagttaacacaaataaagacagcaatgatg

SEQ ID NO: 269 - DNA, 100 bp, Artificial Sequence
  synthesized
acagtggccgtgcacagaagctgttgtactcatgaagaacatatgaaatgcctatgatatttcagccattaccttgtcatgtggctcttgcaaggtggtc

SEQ ID NO: 270 - DNA, 100 bp, Artificial Sequence
  synthesized
cctatgatatttcagccattaccttgtcatgtggctcttgcaaggtggtcaggatgtgcaatgccggctgagagctggtttccaggtaataatccaccaa

SEQ ID NO: 271 - DNA, 100 bp, Artificial Sequence
  synthesized
aggatgtgcaatgccggctgagagctggtttccaggtaataatccaccaaggtgtttacaagcatagggccacggtctaaatcaagaaaagggcaatgga

SEQ ID NO: 272 - DNA, 100 bp, Artificial Sequence
  synthesized
ggtgtttacaagcatagggccacggtctaaatcaagaaaagggcaatggatgatacttattccccttaacatcctaaatttaccttacacagcttcctgt

SEQ ID NO: 273 - DNA, 100 bp, Artificial Sequence
  synthesized
aggattctagtggctctaaagtcaatctcttctttctagaagataagctaaaaaggatattattttgctaaccagaattgaggttctctttaaagacagc

SEQ ID NO: 274 - DNA, 100 bp, Artificial Sequence
  synthesized
aaaaggatattattttgctaaccagaattgaggttctctttaaagacagctgtcacgtcgtcccgcacacccagcatgggggagtccagcatggcaagaa

SEQ ID NO: 275 - DNA, 100 bp, Artificial Sequence
  synthesized
tgtcacgtcgtcccgcacacccagcatgggggagtccagcatggcaagaagctccccgacatttgcttgttgggccattctctcgctcgaaggcgctgtg

SEQ ID NO: 276 - DNA, 100 bp, Artificial Sequence
  synthesized
gctccccgacatttgcttgttgggccattctctcgctcgaaggcgctgtgctggctccaggacgtgtgctacaggttctgaaggttcttcattggggcca

(19) United States
(12) Patent Application Publication
(10) Pub. No.: US 2023/0151418 A1
(43) Pub. Date: May 18, 2023

(54) JOINT DETECTION METHOD FOR LYMPHANGIOLEIO-MYOMATOSIS AND USE THEREOF

(71) Applicant: Guangzhou KingMed Diagnostics Group Co., Ltd., Guangzhou, Guangdong (CN)

(72) Inventors: Xiaohua OU, Guangzhou, Guangdong (CN); Feifei LIU, Guangzhou, Guangdong (CN); Mingming SUN, Guangzhou, Guangdong (CN); Changming HU, Guangzhou, Guangdong (CN); Junhao DENG, Guangzhou, Guangdong (CN); Shihui YU, Guangzhou, Guangdong (CN); Weiwei ZHAO, Guangzhou, Guangdong (CN); Xiaoqiang HUANG, Guangzhou, Guangdong (CN); Chunhui WANG, Guangzhou, Guangdong (CN)

(21) Appl. No.: 17/784,708

(22) PCT Filed: Dec. 27, 2019
(86) PCT No.: PCT/CN2019/129173

§ 371 Date: Jun. 13, 2022

(30) Foreign Application Priority Data
Dec. 13, 2019 (CN) 201911281998.6

(51) Int. Cl. C12Q 1/6858; C12Q 1/6886

(52) U.S. Cl. CPC C12Q 1/6858; C12Q 1/6886; C12Q 2600/156; C12Q 2600/16

(57) ABSTRACT

A joint detection method for lymphangioleio-myomatosis and a use thereof is provided. The method includes the following steps: performing Target Sequencing based Hybridization capture: a Panel is designed for the whole coding regions of TSC1 and TSC2 genes highly related to LAM and mutation genes closely related to solid tumors to construct a gDNA library, and sequencing is performed on a machine after a hybrid capture; sorting: the above sequencing data are processed and analyzed by bioinformatics; performing a supplementary detection by CMA if the TSC1 and TSC2 genes are detected to be negative; performing a supplementary detection by MLPA if a one-hit locus is detected; and performing a supplementary detection by Sanger method if a locus is detected to be a undefined locus derived from either a somatic mutation or a germline mutation. This method improves the positive mutation detection rate of LAM patients.

### CROSS-REFERENCE TO RELATED APPLICATIONS

This application claims priority to PCT Application No. PCT/CN2019/129173, having a filing date of Dec. 27, 2019, which is based on Chinese Application No. 201911281998.6, having a filing date of Dec. 13, 2019, the entire contents both of which are hereby incorporated by reference.

### SEQUENCE LISTING

This application includes a separate sequence listing in compliance with the requirement of 37 C.F.R.§.§ 1.824(a)(2)-1.824 (a)(6) and 1.824 (b), submitted under the file name “0102US01_Sequence_Listing”, created on Jun. 13, 2022, having a file size of 71.5 KB, the contents of which are hereby incorporated by reference.

### FIELD OF TECHNOLOGY

The following relates to the technical field of genetic detection, particularly, it relates to a joint detection method for lymphangioleio-myomatosis and a use thereof.

### BACKGROUND

Lymphangioleio-myomatosis (LAM) is a rare multisystem neoplastic disease that almost exclusively occurs in women. The main symptom of such disease is a disseminated thin-walled cystic lesions in the lung, which has progressed slowly. In the early stages, LAM patients experience mild symptoms, usually with gradually increasing dyspnea symptom, recurrent pneumothorax and chylothorax. About 60% to 70% of the patients develop a pneumothorax at some stages, while about 30% of the patients have chylothorax. The conventional extrapulmonary manifestation includes angiomyolipoma of the kidney, blockage of retroperitoneal lymphangion and blockage of pelvic lymph node, etc.

LAMs can be divided into two types, i.e., Sporadic LAM (S-LAM) and LAM associated with Tuberous Sclerosis Complex (TSC-LAM). The pathogenesis of LAM is unknown yet, but currently, it can be considered that patients suffering from LAM have gene mutations of TSC1 and TSC2, which result in continually activating cellular signaling pathway mediated by mammalian target of rapamycin (mTOR). Sirolimus can effectively inhibit the mTOR pathway and thus become an effective medicament for the treatment of LAM. It is also the first drug approved by FDA to treat LAM. And, it is reported that some LAM patients administrated with Sirolimus show improved lung function and decreased serum level of vascular endothelial growth factor D (VEGF-D) levels, and thus the symptoms of these patients are relieved and their life quality is improved; some LAM patients administrated with Sirolimus show continual exacerbation for the symptoms. Therefore, the primary cause of the difference in Sirolimus response is unclear. It is also shown in the researches that gene mutations of TSC1 and TSC2 cannot be detected in some patients, indicating that there may be another mechanism to participate in the occurrence and development of LAM.

Thus, it is significant for the diagnosis and treatment of LAM to develop a perfect LAM gene detection method and map a genetic mutation profile of a large samples for LAM patients so as to fully understand the pathogenesis of LAM.

### SUMMARY

An aspect relates to a joint detection method for lymphangioleio-myomatosis and a use thereof. In the method, a probe capture method is firstly used to obtain coding regions sequences of TSC1/TSC2 genes and hotspot regions sequences of core genes related to the occurrence and development of tumors; and then paired-end sequencing is conducted on Illumina sequencing platform to detect sequence variant in the targeted region of samples; meanwhile, supplementary detections and result verification are performed in the methods, such as CMA, MLPA and Sanger, etc. to improve positive variant detection rate of LAM patients. Further, auxiliary diagnosis and pre-conception genetic counseling can be performed according to the detection results.

A joint detection method for lymphangioleio-myomatosis, comprises the steps as follows:

performing Target Sequencing based Hybridization capture: A Panel is designed for whole coding regions of TSC1 and TSC2 genes highly related to LAM and mutation genes closely related to solid tumors to construct a Genomic DNA (gDNA) library; sequencing is performed on a machine after a hybrid capture;

sorting: data obtained from the above sequencing is processed and analyzed through bioinformatics; when TSC1 and TSC2 genes are detected to be negative or there is only a one-hit mutated locus, a supplementary detection is performed by chromosomal microarray analysis (CMA) and Multiplex ligation-dependent probe amplification (MLPA); when a locus is detected to be an undefined locus originated from either a somatic mutation or a germline mutation, the locus can be verified by Sanger sequencing;

performing CMA to obtain loss of heterozygosity (LOH) and copy number variations; and

performing MLPA to obtain large fragment insertions and deletions; and

performing Sanger sequencing to test leukocyte samples corresponding to samples to be tested after taking the leukocyte samples, and to further determine whether the samples are S-LAM or TSC-LAM.

It has been determined that TSC1 and TSC2 genes are larger, including 23 and 42 exons, respectively, 0 with total coding region length of 9 kb, and have complex sequence regions. In conventional methods, such as Sanger sequencing, TSC1 and TSC2 genes are required to be segmented and amplified for several times and then sequenced, which consumes more workload, money, and samples. For the poor-quality Formalin-Fixed Paraffin-Embedded (FFPE) samples, the conventional amplification fails to achieve full coverage.

In the meanwhile, the research showed that the frequency of the somatic mutation for TSC1 and TSC2 genes was less than 10% for about 50% of LAM patients and the detection sensitivity of the conventional Sanger method was above 10%, leading to detection omission. Further, the occurrence and development of LAM is based on a Double-hit model, and the mutations of LAM occur in multiple forms, such as LOH and large fragment insertion and deletion. Therefore, a single detection method cannot meet the requirements of efficient detection. As LAM is heritable and sporadic, the hereditary patients with unobvious symptoms are often clinically misdiagnosed as sporadic LAM.

Based on the above research basis, the above joint detection method has been provided. In addition to the gene mutations of TSC1 and TSC2, another mechanism, which may be involved in the occurrence and development of LAM, is also taken into consideration in the method. Therefore, the detection of other tumor-related genes is included in the comprehensive detection of above-mentioned LAM, and meanwhile, methods such as CMA, MLPA, and Sanger, etc. are used for auxiliary diagnosis and result verification, thereby improving the positive mutation detection rate of LAM patients. Further, auxiliary diagnosis and pre-conception genetic counseling can be performed according to the detected result.

In one example, in the step of Target Sequencing based Hybridization capture, the Panel is designed to cover the following genes: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

With references to databases, such as COSMIC and TCGA etc., in combination with the latest guideline/consensus of National Comprehensive Cancer Network (NCCN), proto-oncogenes and tumor suppressor genes closely related to the occurrence and development of solid tumors are screened out and combined to form the above Panel, a gene panel.

In one example, in the step of Target Sequencing based Hybridization capture, probe sequences for the TSC1 and TSC2 genes include SEQ ID NO: 1 to SEQ ID NO: 276.

For the above-mentioned Panel design, the whole coding regions of TSC1 and TSC2 genes, which are basically highly related to LAM, are designed in a shingled form to ensure target regions of the two genes to be covered at least 2 times, with each probe length of 100 bp, thereby further increasing the detection rate.

In one example, in the step of Target Sequencing based Hybridization capture, the sequencing depth is more than 1000×. A mutated locus is identified at a mutation frequency of more than 1%, to avoid detection omission at a low mutation frequency.

It is another aspect to provide a use of the joint detection method for LAM in a study of pathogenesis of LAM and/or in a diagnosis and treatment of LAM.

In one example, it relates to a use of a specific detection reagent in the joint detection method in a preparation of a diagnostic reagent or a diagnostic equipment for jointly detecting LAM.

The present disclosure further discloses a joint detection kit for LAM, including a Panel covering the following genes: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

In one example, probe sequences of the Panel include SEQ ID NO: 1 to SEQ ID NO: 276.

In one example, the joint detection kit further includes an agent for CMA. It should be understood that the agent for CMA can be selected according to a practical experimental requirement, such as OncoScan® CNV FFRE Assay kit.

In one example, the joint detection kit further includes multiplex ligation-dependent probes for MLPA. It should be understood that the probes can be selected according to practical experimental requirements, 0 such as TSC1 and TSC2 probes from MRC-Holland.

It is another aspect to provide a joint detection system for LAM, comprising the modules as follows:

a detection module, comprising a module of Target Sequencing based Hybridization capture, a module of CMA, a module of MLPA, and a module of Sanger sequencing, wherein the module of Target Sequencing based Hybridization capture comprises a Panel designed for whole coding regions of TSC1 and TSC2 genes highly related to LAM and mutated genes closely related to solid tumors; and

an analysis module, firstly obtaining a detection result of Target Sequencing based Hybridization capture; requesting the module of chromosomal microarray analysis (CMA) and the module of Multiplex ligation-dependent probe amplification (MLPA) to perform a supplementary detection when TSC1 and TSC2 genes are detected to be negative or there is only a one-hit mutated locus; requesting the module of Sanger sequencing to verify the undefined locus when a locus is detected to be an undefined locus originated from either a somatic mutation or a germline mutation; and then analyzing and judging detection results from the detection modules, to draw a joint detection result of LAM.

In one example, the Panel covers genes as follows: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

In one example, in the module of Target Sequencing based Hybridization capture, probe sequences for the TSC1 and TSC2 genes include SEQ ID NO: 1 to SEQ ID NO: 276.

It should be understood, that the above-mentioned joint detection system can be a combination of equipments, instruments, or agents that can perform Target Sequencing based Hybridization capture, CMA, MPLA, and Sanger sequencing, as long as the functions of the system can be achieved. For example, the conventional equipments and instruments can be used with specific Panel of the present disclosure, to achieve the purpose of obtaining the whole coding regions sequences of TSC1 and TSC2 genes and the hotspot region sequences of the core genes related to the occurrence and development of the tumor, and performing supplementary detections and result verifications in the methods, such as CMA, MLPA, Sanger sequencing, etc. at the same time.

Compared with the conventional art, the present disclosure has the following benefits:

The present disclosure provides a joint detection method for lymphangioleio-myomatosis. In the method, a probe capture method is firstly used to obtain coding regions sequences of TSC1/TSC2 genes and hotspot regions sequences of the core gene related to the occurrence and development of the tumor; and then paired-end sequencing is conducted on Illumina sequencing platform to detect sequence variant in the targeted region of samples; meanwhile supplementary detections and result verifications are performed in the methods, such as CMA, MLPA and Sanger, to improve the positive variant detection rate of LAM patients. Further, auxiliary diagnosis and pre-conception genetic counseling can be performed according to the detection results.

In addition, for poor-quality FFPE samples, TSC1 and TSC2 genes and the hotspot regions of the core gene related to solid tumor can be perfectly obtained and sequenced in a single experiment, by using a 2×100 bp probe design scheme, combined with a liquid-phase hybridization capture method.

The present disclosure provides a joint detection kit for LAM, which can be used for detecting coding regions sequences of TSC1 and TSC2 genes and hotspot regions sequences of the core gene related to the occurrence and development of the tumor. The joint detection kit also includes an agent for CMA reagent and/or multiplex ligation-dependent probes for MLPA, etc., which can be selected according to the requirements. And meanwhile, supplementary detections and result verifications can be performed with the methods, such as CMA, MLPA, and Sanger sequencing to improve the positive mutation detection rate of LAM patients. Further, auxiliary diagnosis and pre-conception genetic counseling can be performed according to the detection results.

The present disclosure provides a joint detection system for LAM, comprising a detection module and an analysis module, wherein the detection module comprises a module of Target Sequencing based Hybridization capture, a module of chromosomal microarray analysis (CMA), a module of Multiplex ligation-dependent probe amplification (MLPA) and a module of Sanger sequencing, etc. The system can be used for detecting coding regions sequences of TSC1 and TSC2 genes and hotspot regions sequences of the core genes related to the occurrence and development of the tumor. In the system, methods, such as CMA, MLPA and Sanger, etc. can be selected according to the requirements, and meanwhile, supplementary detections and result verification can be performed with the methods, such as CMA, MLPA and Sanger, etc. to improve the positive mutation detection rate of LAM patients. Further, auxiliary diagnosis and pre-conception genetic counseling can be performed according to the detection results.

### BRIEF DESCRIPTION

Some of examples will be described in detail, with reference to the following figures, wherein like designations denote like members, wherein:

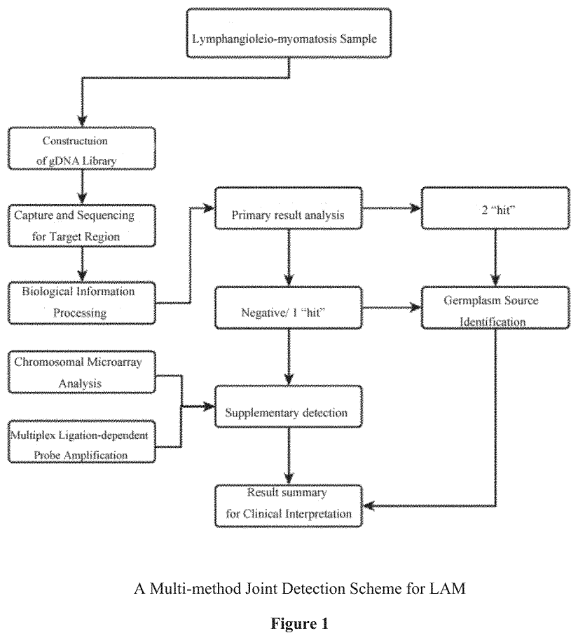

FIG. 1 shows a flow diagram of a multi-method joint detection scheme for LAM;

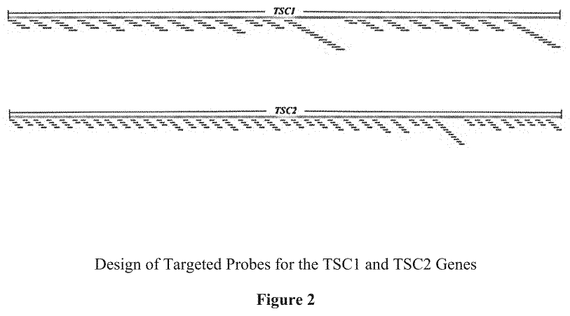

FIG. 2 shows a design of targeted probes for the TSC1 and TSC2 genes;

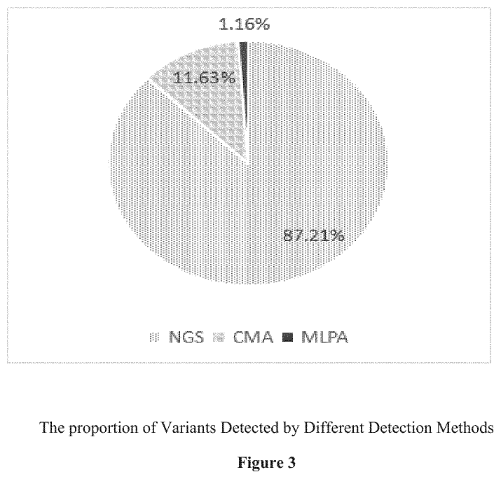

FIG. 3 shows a pie chart of the proportion of variants detected by different detection methods;

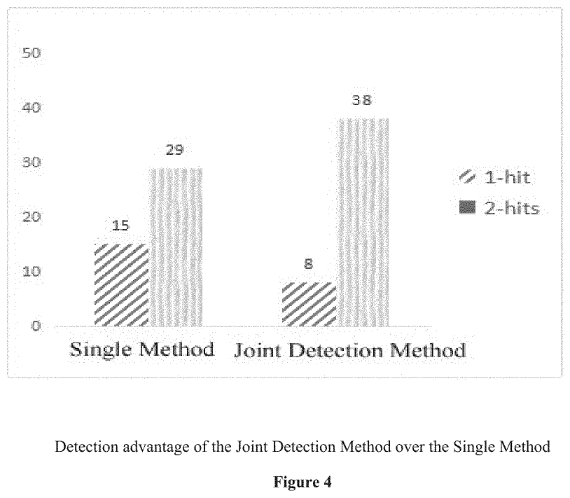

FIG. 4 shows a detection advantage of the joint detection method over the single method; and

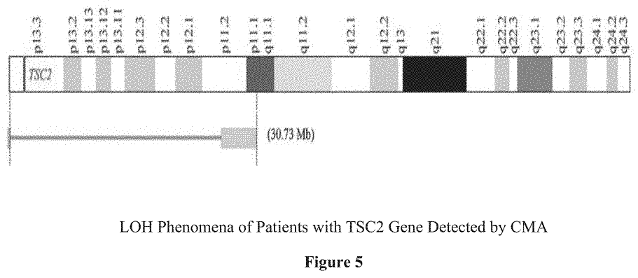

FIG. 5 shows LOH phenomena of patients with TSC2 gene detected by CMA.

### DETAILED DESCRIPTION

For better understanding of the present disclosure, the present disclosure will be fully described below with reference to the relevant accompanying figures. Preferred embodiments are shown in the figures. However, the present disclosure can be implemented in many different forms and is not limited to the embodiments described herein. Rather, these embodiments are provided for the purpose of making the disclosed contents of the present disclosure more thorough and complete.

Unless otherwise defined, all technical and scientific terms used herein have the same meaning as those normally understood by one skilled in the art in the technical field of the present disclosure. The terms used in the description of the present disclosure herein are only for the purpose of describing embodiments, and are not intended to limit the present disclosure. The term “and/or” used herein comprises anyone or all combinations of one or more corresponding items listed herein.

### Example 1

LAM samples were detected as follows, according to a multi-method joint detection scheme for LAM, shown in FIG. 1.

I. Performing Target Sequencing Based Hybridization Capture, a Next-Generation Sequencing (NGS), with Probes:

1. Panel Design

The whole coding regions of TSC1 and TSC2 genes, which are basically highly related to LAM, were designed in a shingled form to ensure that the target regions of these two genes were covered at least 2 times, with each probe length of 100 bp, wherein the probes for TSC1 and TSC2 genes were designed as shown in FIG. 2.

At the same time, with reference to databases, such as COSMIC, TCGA, etc., in combination with the latest guidelines/consensus of NCCN, proto-oncogenes and tumor suppressor genes that were closely related to the occurrence and development of solid tumors were screened out, while probes were designed according to conventional method, that is, the probes were connected to the target region in an end-to-end manner at 1-fold coverage. The gene panel was listed as follows:

TABLE 1
Panel's Gene List

| Coverage | Gene list | | | | | |
|---|---|---|---|---|---|---|
| Whole Coding | ALDH1 | EGFR | FLT3 | MYC | PTEN | SDHD |
| Regions | AQP9 | ERBB2 | HRAS | MYCN | RET | TP53 |
| (40 genes) | AR | ESR1 | KIT | NF1 | RICTOR | TSC1 |
| | ATRX | FGFR1 | KRAS | NRAS | RUNX1 | TSC2 |
| | BCL2 | FGFR2 | MDM2 | PDGFRA | SDHA | VHL |
| | BRAF | FGFR3 | MAP2K1 | PGR | SDHB | |
| | CCND1 | FGFR4 | MET | POLE | SDHC | |
| Hotspot | ABL1 | CDKN2A | FBXW7 | IDH2 | NOTCH1 | SMAD4 |
| mutations | AKT1 | CSF1R | GNA11 | JAK2 | NPM1 | SMARCB1 |
| (36 genes) | ALK | CTNNB1 | GNAQ | JAK3 | PIK3CA | SMO |
| | APC | DDR2 | GNAS | KDR | PTPN11 | SRC |
| | ATM | ERBB4 | HNF1A | MLH1 | RB1 | STK11 |
| | CDH1 | EZH2 | IDH1 | MPL | ROS1 | TET2 |

TABLE 2
Design Table of Probes for TSC1 and TSC2 Genes

| SEQ | | | | | |
|---|---|---|---|---|---|
| ID | | | | | |
| NO: | sequences | Chr | Start | Stop | Group |
| 1 | GTCCCCATTCCTGTTTCGTTTGCACAGAGGGGTTTTCTGGTGCGT | 16 | 20985 | 2098 | TSC2 |
| | CCTGGTCCACCATGGCCAAACCAACAAGCAAAGATTCAGGCTTG | | 60 | 660 | |
| | AAGGAGAAGTT | | | | |
| 2 | TCCACCATGGCCAAACCAACAAGCAAAGATTCAGGCTTGAAGG | 16 | 20986 | 2098 | TSC2 |
| | AGAAGTTTAAGATTCTGTTGGGACTGGGAACACCGAGGCCAAAT | | 10 | 710 | |
| | CCCAGGTCTGCAG | | | | |
| 3 | TAAGATTCTGTTGGGACTGGGAACACCGAGGCCAAATCCCAGGT | 16 | 20986 | 2098 | TSC2 |
| | CTGCAGAGGGTAAACAGACGGAGTTTATCATCACCGCGGAAAT | | 60 | 760 | |
| | ACTGAGAGTGAGT | | | | |
| 4 | AGGGTAAACAGACGGAGTTTATCATCACCGCGGAAATACTGAG | 16 | 20987 | 2098 | TSC2 |
| | AGTGAGTGAGCTACCTGTGTCTTTGCTAGGCTAGAGGGAAATGC | | 10 | 810 | |
| | AGAGAAGGCTGGG | | | | |
| 5 | TTAAGGAGACCGTGGCCTGAGCACTGGCCCCTTTTTCTTCTTTCA | 16 | 21003 | 2100 | TSC2 |
| | TCTCTCTCCAGGAACTGAGCATGGAATGTGGCCTCAACAATCGC | | 44 | 444 | |
| | ATCCGGATGAT | | | | |
| 6 | CTCCAGGAACTGAGCATGGAATGTGGCCTCAACAATCGCATCCG | 16 | 21003 | 2100 | TSC2 |
| | GATGATAGGGCAGATTTGTGAAGTCGCAAAAACCAAGAAATTT | | 94 | 494 | |
| | GAAGAGGTAGGTT | | | | |
| 7 | AGGGCAGATTTGTGAAGTCGCAAAAACCAAGAAATTTGAAGAG | 16 | 21004 | 2100 | TSC2 |
| | GTAGGTTTATCCAGTTGAGCTACTAGAGAGAGGCACGTAGACTA | | 44 | 544 | |
| | TTCAGAGCCTGAG | | | | |
| 8 | TGCTCCAGTTGCCGGGGCCAGGGTTCTTGGAGAGCACATCCTCA | 16 | 21032 | 2103 | TSC2 |
| | CCGCTGTCCCCTCTGCTGGTGACAGCACGCAGTGGAAGCACTCT | | 73 | 373 | |
| | GGAAGGCGGTCG | | | | |
| 9 | TCCCCTCTGCTGGTGACAGCACGCAGTGGAAGCACTCTGGAAGG | 16 | 21033 | 2103 | TSC2 |
| | CGGTCGCGGATCTGTTGCAGCCGGAGCGGCCGCTGGAGGCCCGG | | 23 | 423 | |
| | CACGCGGTGCTG | | | | |
| 10 | CGGATCTGTTGCAGCCGGAGCGGCCGCTGGAGGCCCGGCACGC | 16 | 21033 | 2103 | TSC2 |
| | GGTGCTGGCTCTGCTGAAGGCCATCGTGCAGGGGCAGGTAAGGC | | 73 | 473 | |
| | CCAGGGCGACGCT | | | | |
| 11 | GCTCTGCTGAAGGCCATCGTGCAGGGGCAGGTAAGGCCCAGGG | 16 | 21034 | 2103 | TSC2 |
| | CGACGCTGGGATGGGTGACGTCAGGCTGCCCACTGACTGTCCTG | | 23 | 523 | |
| | TCCCTGCTGGGCC | | | | |
| 12 | CCGTGTGGGCGACGCTGGCAGGCTCTGCTGATCCTGTGGCTTTT | 16 | 21042 | 2104 | TSC2 |
| | GTCTTTAGGGCGAGCGTTTGGGGGTCCTCAGAGCCCTCTTCTTTA | | 44 | 344 | |
| | AGGTCATCAAG | | | | |
| 13 | AGGGCGAGCGTTTGGGGGTCCTCAGAGCCCTCTTCTTTAAGGTC | 16 | 21042 | 2104 | TSC2 |
| | ATCAAGGATTACCCTTCCAACGAAGACCTTCACGAAAGGCTGGA | | 94 | 394 | |
| | GGTTTTCAAGGC | | | | |
| 14 | GATTACCCTTCCAACGAAGACCTTCACGAAAGGCTGGAGGTTTT | 16 | 21043 | 2104 | TSC2 |
| | CAAGGCCCTCACAGACAATGGGAGACACATCACCTACTTGGAG | | 44 | 444 | |
| | GAAGAGCTGGGTG | | | | |
| 15 | CCTCACAGACAATGGGAGACACATCACCTACTTGGAGGAAGAG | 16 | 21043 | 2104 | TSC2 |
| | CTGGGTGGGTGCCACCTTGGGTTGGAGGTTTCTCTGGCCTTGAC | | 94 | 494 | |
| | GATCAAGTGTAAC | | | | |
| 16 | GGAGGGGGAGGTGAGTGGGAGATGTAGATTCGGCGTCCTCGCA | 16 | 21053 | 2105 | TSC2 |
| | AACTGCCGCCGCTTCTCCCCCAGCTGACTTTGTCCTGCAGTGGAT | | 36 | 436 | |
| | GGATGTTGGCTT | | | | |
| 17 | GCCGCTTCTCCCCCAGCTGACTTTGTCCTGCAGTGGATGGATGTT | 16 | 21053 | 2105 | TSC2 |
| | GGCTTGTCCTCGGAATTCCTTCTGGTGCTGGTGAACTTGGTCAAA | | 86 | 486 | |
| | TTCAATAGCT | | | | |
| 18 | GTCCTCGGAATTCCTTCTGGTGCTGGTGAACTTGGTCAAATTCAA | 16 | 21054 | 2105 | TSC2 |
| | TAGCTGTTACCTCGACGAGTACATCGCAAGGATGGTTCAGTAAG | | 36 | 536 | |
| | AAAAGAATTGA | | | | |
| 19 | GTTACCTCGACGAGTACATCGCAAGGATGGTTCAGTAAGAAAAG | 16 | 21054 | 2105 | TSC2 |
| | AATTGAGATCCTGTTCTGATAATGGTCCTAAGTTCAGCTCCGCA | | 86 | 586 | |
| | GTGAATAAAGTT | | | | |
| 20 | CTCGGCCATCCAGGCAGTGCTGCCGGGACTGAGCTCGGTGCTCC | 16 | 21061 | 2106 | TSC2 |
| | CTGCAGGATGATCTGTCTGCTGTGCGTCCGGACCGCGTCCTCTGT | | 46 | 246 | |
| | GGACATAGAGG | | | | |
| 21 | GATGATCTGTCTGCTGTGCGTCCGGACCGCGTCCTCTGTGGACAT | 16 | 21061 | 2106 | TSC2 |
| | AGAGGTCAGTGCCTCCCCTCCCCAGGGCCGGCCCATTTCACCCT | | 96 | 296 | |
| | GGTTTCTGGGA | | | | |
| 22 | TGTCCTCTCCTGTGGGGAGGAGCTGGGGTAGGACGGGCGTGAGC | 16 | 21065 | 2106 | TSC2 |
| | CGTCTCCCTCTCCACCAGGTCTCCCTGCAGGTGCTGGACGCCGTG | | 82 | 682 | |
| | GTCTGCTACAA | | | | |
| 23 | CCTCTCCACCAGGTCTCCCTGCAGGTGCTGGACGCCGTGGTCTG | 16 | 21066 | 2106 | TSC2 |
| | CTACAACTGCCTGCCGGCTGAGAGCCTCCCGCTGTTCATCGTTAC | | 32 | 732 | |
| | CCTCTGTCGCA | | | | |
| 24 | CTGCCTGCCGGCTGAGAGCCTCCCGCTGTTCATCGTTACCCTCTG | 16 | 21066 | 2106 | TSC2 |
| | TCGCACCATCAACGTCAAGGAGCTCTGCGAGCCTTGCTGGAAGG | | 82 | 782 | |
| | TGGGGTTTCTG | | | | |
| 25 | CCATCAACGTCAAGGAGCTCTGCGAGCCTTGCTGGAAGGTGGGG | 16 | 21067 | 2106 | TSC2 |
| | TTTCTGAAACTGCTCTGGAAGGTTCCTGAGAGCACATGGATGGG | | 32 | 832 | |
| | ACAAGGGCCATC | | | | |
| 26 | CGGGACTGGGGCTGGGGGCAGGGCTTATGCCTGCCAGCCCCTGA | 16 | 21070 | 2107 | TSC2 |
| | CACGCATTGTGTCTCGCAGCTGATGCGGAACCTCCTTGGCACCC | | 42 | 142 | |
| | ACCTGGGCCACA | | | | |
| 27 | TTGTGTCTCGCAGCTGATGCGGAACCTCCTTGGCACCCACCTGG | 16 | 21070 | 2107 | TSC2 |
| | GCCACAGCGCCATCTACAACATGTGCCACCTCATGGAGGACAGG | | 92 | 192 | |
| | TGAGTGTGGTGG | | | | |
| 28 | GCGCCATCTACAACATGTGCCACCTCATGGAGGACAGGTGAGTG | 16 | 21071 | 2107 | TSC2 |
| | TGGTGGGTGGGGCGCAGGGCAGTGGAGGCCAGCACAGCCCTCG | | 42 | 242 | |
| | GGGCAGCTCCAGT | | | | |
| 29 | CCATGGCGGACCCTGGGACAGGGCCCTGCTCACATTCCGTCTCT | 16 | 21086 | 2108 | TSC2 |
| | CTGGGGAACACTTTTAGAGCCTACATGGAGGACGCGCCCCTGCT | | 86 | 786 | |
| | GAGAGGAGCCGT | | | | |
| 30 | AACACTTTTAGAGCCTACATGGAGGACGCGCCCCTGCTGAGAGG | 16 | 21087 | 2108 | TSC2 |
| | AGCCGTGTTTTTTGTGGGCATGGCTCTCTGGGGAGCCCACCGGC | | 36 | 836 | |
| | TCTATTCTCTCA | | | | |
| 31 | GTTTTTTGTGGGCATGGCTCTCTGGGGAGCCCACCGGCTCTATTC | 16 | 21087 | 2108 | TSC2 |
| | TCTCAGGAACTCGCCGACATCTGTGTTGCCATCATTTTACCAGGT | | 86 | 886 | |
| | AAGGCGGTTT | | | | |
| 32 | GGAACTCGCCGACATCTGTGTTGCCATCATTTTACCAGGTAAGG | 16 | 21088 | 2108 | TSC2 |
| | CGGTTTCTGTGTGCAGTGAGCTGGCAGGAACGGGAGAGCTCCCC | | 36 | 936 | |
| | TCACGCCTGCCC | | | | |
| 33 | CACAGCAAGCAAGCAGCTCTGACCCTGTGTGCTGGCCGGGCTCG | 16 | 21106 | 2110 | TSC2 |
| | TGTTCCAGGCCATGGCATGTCCGAACGAGGTGGTGTCCTATGAG | | 18 | 718 | |
| | ATCGTCCTGTCC | | | | |
| 34 | AGGCCATGGCATGTCCGAACGAGGTGGTGTCCTATGAGATCGTC | 16 | 21106 | 2110 | TSC2 |
| | CTGTCCATCACCAGGCTCATCAAGAAGTATAGGAAGGAGCTCCA | | 68 | 768 | |
| | GGTGGTGGCGTG | | | | |
| 35 | ATCACCAGGCTCATCAAGAAGTATAGGAAGGAGCTCCAGGTGG | 16 | 21107 | 2110 | TSC2 |
| | TGGCGTGGGACATTCTGCTGAACATCATCGAACGGCTCCTTCAG | | 18 | 818 | |
| | CAGCTCCAGGTGG | | | | |
| 36 | GGACATTCTGCTGAACATCATCGAACGGCTCCTTCAGCAGCTCC | 16 | 21107 | 2110 | TSC2 |
| | AGGTGGGGTGGGGGCAGGAGCTCCGGGGAGCACCGGGAACCCA | | 68 | 868 | |
| | GACAGGCAGGCTC | | | | |
| 37 | GCCAAGTCCATGTGGGGAGTGGAAGTCAGCCTGTGTCATCGTGC | 16 | 21118 | 2111 | TSC2 |
| | CTGGTACTGCAGACCTTGGACAGCCCGGAGCTCAGGACCATCGT | | 15 | 915 | |
| | CCATGACCTGTT | | | | |
| 38 | CTGCAGACCTTGGACAGCCCGGAGCTCAGGACCATCGTCCATGA | 16 | 21118 | 2111 | TSC2 |
| | CCTGTTGACCACGGTGGAGGAGCTGTGTGACCAGAACGAGTTCC | | 65 | 965 | |
| | ACGGGTCTCAGG | | | | |
| 39 | GACCACGGTGGAGGAGCTGTGTGACCAGAACGAGTTCCACGGG | 16 | 21119 | 2112 | TSC2 |
| | TCTCAGGAGAGATACTTTGAACTGGTGGAGAGATGTGCGGACCA | | 15 | 015 | |
| | GAGGCCTGTGAGA | | | | |
| 40 | AGAGATACTTTGAACTGGTGGAGAGATGTGCGGACCAGAGGCC | 16 | 21119 | 2112 | TSC2 |
| | TGTGAGACCCCCTCCTGGGTGGGGCCTTTGGGCTTTGGCTGGTG | | 65 | 065 | |
| | GGGAGGGGCCGGG | | | | |
| 41 | GACCAGCAGCCCAGTGTGGAGAAGGAGAGCGCCGGAGGGGCAG | 16 | 21124 | 2112 | TSC2 |
| | AGGGGCAACACCGGCTCTTCTTTTGACAGGAGTCCTCCCTCCTG | | 25 | 525 | |
| | AACCTGATCTCCT | | | | |
| 42 | ACACCGGCTCTTCTTTTGACAGGAGTCCTCCCTCCTGAACCTGAT | 16 | 21124 | 2112 | TSC2 |
| | CTCCTATAGAGCGCAGTCCATCCACCCGGCCAAGGACGGCTGGA | | 75 | 575 | |
| | TTCAGAACCTG | | | | |
| 43 | ATAGAGCGCAGTCCATCCACCCGGCCAAGGACGGCTGGATTCAG | 16 | 21125 | 2112 | TSC2 |
| | AACCTGCAGGCGCTGATGGAGAGATTCTTCAGGTAGGGGGTCCT | | 25 | 625 | |
| | CTGTAGCCTTGC | | | | |
| 44 | CAGGCGCTGATGGAGAGATTCTTCAGGTAGGGGGTCCTCTGTAG | 16 | 21125 | 2112 | TSC2 |
| | CCTTGCCTGGCACCTGGAGCCTGGCCCTGTCTCTGTCTGGGGCCC | | 75 | 675 | |
| | ACCCGGGCTGG | | | | |
| 45 | CTGGTGTGGGGCTGTGGCCGGGCACTCCCCACCCGCCCCAGCAG | 16 | 21129 | 2113 | TSC2 |
| | GCTGCCGTCCCGCAGGAGCGAGTCCCGAGGCGCCGTGCGCATCA | | 13 | 013 | |
| | AGGTGCTGGACG | | | | |
| 46 | GTCCCGCAGGAGCGAGTCCCGAGGCGCCGTGCGCATCAAGGTG | 16 | 21129 | 2113 | TSC2 |
| | CTGGACGTGCTGTCCTTTGTGCTGCTCATCAACAGGCAGTTCTAT | | 63 | 063 | |
| | GAGGTGCGTGTC | | | | |
| 47 | TGCTGTCCTTTGTGCTGCTCATCAACAGGCAGTTCTATGAGGTGC | 16 | 21130 | 2113 | TSC2 |
| | GTGTCCAGGCGGCCGCAGCTGGGGGCTCAGGGCTATTTCTCCGT | | 13 | 113 | |
| | GGGCGGGCTGT | | | | |
| 48 | GGAATTGGAAGTGTCACGAGATGTGGCCCTCGTTGGGCTGGCGC | 16 | 21142 | 2114 | TSC2 |
| | TCATTGGCCTCCCTTGTGCCTGTGCAGGAGGAGCTGATTAACTC | | 01 | 301 | |
| | AGTGGTCATCTC | | | | |
| 49 | GCCTCCCTTGTGCCTGTGCAGGAGGAGCTGATTAACTCAGTGGT | 16 | 21142 | 2114 | TSC2 |
| | CATCTCGCAGCTCTCCCACATCCCCGAGGATAAAGACCACCAGG | | 51 | 351 | |
| | TCCGAAAGCTGG | | | | |
| 50 | GCAGCTCTCCCACATCCCCGAGGATAAAGACCACCAGGTCCGAA | 16 | 21143 | 2114 | TSC2 |
| | AGCTGGCCACCCAGTTGCTGGTGGACCTGGCAGAGGGCTGCCAC | | 01 | 401 | |
| | ACACACCACTTC | | | | |
| 51 | CCACCCAGTTGCTGGTGGACCTGGCAGAGGGCTGCCACACACAC | 16 | 21143 | 2114 | TSC2 |
| | CACTTCAACAGCCTGCTGGACATCATCGAGAAGGTGAGAGCCGT | | 51 | 451 | |
| | TGTACCCGGGGC | | | | |
| 52 | AACAGCCTGCTGGACATCATCGAGAAGGTGAGAGCCGTTGTACC | 16 | 21144 | 2114 | TSC2 |
| | CGGGGCCGGGTGCTAGCGTGCCAGAGCTCCGTGGGCAGCAATG | | 01 | 501 | |
| | GCCTCTGGGCCCT | | | | |
| 53 | AGTGTTCTCACGGCTGCTGACTCAGAACCATGAGCCTGTGTGTA | 16 | 21154 | 2115 | TSC2 |
| | AGTCCTGGCCTTCTCTTCAAAGGTGATGGCCCGCTCCCTCTCCCC | | 53 | 553 | |
| | ACCCCCGGAGC | | | | |
| 54 | GGCCTTCTCTTCAAAGGTGATGGCCCGCTCCCTCTCCCCACCCCC | 16 | 21155 | 2115 | TSC2 |
| | GGAGCTGGAAGAAAGGGATGTGGCCGCATACTCGGCCTCCTTGG | | 03 | 603 | |
| | AGGATGTGAAG | | | | |
| 55 | TGGAAGAAAGGGATGTGGCCGCATACTCGGCCTCCTTGGAGGAT | 16 | 21155 | 2115 | TSC2 |
| | GTGAAGACAGCCGTCCTGGGGCTTCTGGTCATCCTTCAGGTGGG | | 53 | 653 | |
| | TGTTCTGCACGA | | | | |
| 56 | ACAGCCGTCCTGGGGCTTCTGGTCATCCTTCAGGTGGGTGTTCTG | 16 | 21156 | 2115 | TSC2 |
| | CACGAGGCCTCTGCTCCCGGGGCGCGCATGGCTAGCGTCCACCA | | 03 | 703 | |
| | GCTGCATCTGC | | | | |
| 57 | GTGCTGTCTTAGGACTGCGTTTTCACCTCCTGCGCCGTGGTGAGC | 16 | 21203 | 2120 | TSC2 |
| | TGCGTCCTCTCTCTGCAGACCAAGCTGTACACCCTGCCTGCAAG | | 93 | 493 | |
| | CCACGCCACGC | | | | |
| 58 | CCTCTCTCTGCAGACCAAGCTGTACACCCTGCCTGCAAGCCACG | 16 | 21204 | 2120 | TSC2 |
| | CCACGCGTGTGTATGAGATGCTGGTCAGCCACATTCAGCTCCAC | | 43 | 543 | |
| | TACAAGCACAGC | | | | |
| 59 | GTGTGTATGAGATGCTGGTCAGCCACATTCAGCTCCACTACAAG | 16 | 21204 | 2120 | TSC2 |
| | CACAGCTACACCCTGCCAATCGCGAGCAGCATCCGGCTGCAGGT | | 93 | 593 | |
| | ATGGTGGCTGGG | | | | |
| 60 | TACACCCTGCCAATCGCGAGCAGCATCCGGCTGCAGGTATGGTG | 16 | 21205 | 2120 | TSC2 |
| | GCTGGGGTTGCGCAGCCAGTTCCTGGGGGCCCAGCCAGGTATCC | | 43 | 643 | |
| | CCGTCTCGGCAG | | | | |
| 61 | GCAGGTGGGACGCCGCCTGTCCTGGGCCTGCACGAGCTTGGCTC | 16 | 21214 | 2121 | TSC2 |
| | TGGCTTTCACCATCCTCTTCCTGACAGGCCTTTGACTTCCTGTTG | | 39 | 539 | |
| | CTGCTGCGGGC | | | | |
| 62 | TCACCATCCTCTTCCTGACAGGCCTTTGACTTCCTGTTGCTGCTG | 16 | 21214 | 2121 | TSC2 |
| | CGGGCCGACTCACTGCACCGCCTGGGCCTGCCCAACAAGGATGG | | 89 | 589 | |
| | AGTCGTGCGGT | | | | |
| 63 | CGACTCACTGCACCGCCTGGGCCTGCCCAACAAGGATGGAGTCG | 16 | 21215 | 2121 | TSC2 |
| | TGCGGTTCAGCCCCTACTGCGTCTGCGACTACATGTACGCGGGA | | 39 | 639 | |
| | CCTCGCCCACGG | | | | |
| 64 | TCAGCCCCTACTGCGTCTGCGACTACATGTACGCGGGACCTCGC | 16 | 21215 | 2121 | TSC2 |
| | CCACGGCCCATGAGGCTCAGGGCGTCAGAGGCGCTGGGGCTGT | | 89 | 689 | |
| | GGTGGCGCTGTTT | | | | |
| 65 | GGGTTGGGAAGAGCCAAGTCTGTTCCGTTCCTGCTGCGGGGACT | 16 | 21217 | 2121 | TSC2 |
| | TGGCCTCAGCTGCTTCTCTTGCTTCTGCAGGGAGCCAGAGAGAG | | 10 | 810 | |
| | GCTCTGAGAAGA | | | | |
| 66 | CAGCTGCTTCTCTTGCTTCTGCAGGGAGCCAGAGAGAGGCTCTG | 16 | 21217 | 2121 | TSC2 |
| | AGAAGAAGACCAGCGGCCCCCTTTCTCCTCCCACAGGGCCTCCT | | 60 | 860 | |
| | GGCCCGGCGCCT | | | | |
| 67 | AGACCAGCGGCCCCCTTTCTCCTCCCACAGGGCCTCCTGGCCCG | 16 | 21218 | 2121 | TSC2 |
| | GCGCCTGCAGGCCCCGCCGTGCGGCTGGGGTCCGTGCCCTACTC | | 10 | 910 | |
| | CCTGCTCTTCCG | | | | |
| 68 | GCAGGCCCCGCCGTGCGGCTGGGGTCCGTGCCCTACTCCCTGCT | 16 | 21218 | 2121 | TSC2 |
| | CTTCCGCGTCCTGCTGCAGTGCTTGAAGCAGGTGAGTGGGGCCG | | 60 | 960 | |
| | GGCAGGGACCAT | | | | |
| 69 | CGTCCTGCTGCAGTGCTTGAAGCAGGTGAGTGGGGCCGGGCAGG | 16 | 21219 | 2122 | TSC2 |
| | GACCATCCGTCCCACGTTGGGCCAGGAGGACAGGGAGCTGCCA | | 10 | 010 | |
| | CCTGCCTGCTGGG | | | | |
| 70 | CTAGCTTCCGCCTCTGTCTCTAGGGTCCAGAAGGCCCTGTCCTGA | 16 | 21221 | 2122 | TSC2 |
| | CGCCTCCTCTCCTCGCAGGAGTCTGACTGGAAGGTGCTGAAGCT | | 78 | 278 | |
| | GGTTCTGGGCA | | | | |
| 71 | CCTCTCCTCGCAGGAGTCTGACTGGAAGGTGCTGAAGCTGGTTC | 16 | 21222 | 2122 | TSC2 |
| | TGGGCAGGCTGCCTGAGTCCCTGCGCTATAAAGTGCTCATCTTT | | 28 | 328 | |
| | ACTTCCCCTTGC | | | | |
| 72 | GGCTGCCTGAGTCCCTGCGCTATAAAGTGCTCATCTTTACTTCCC | 16 | 21222 | 2122 | TSC2 |
| | CTTGCAGTGTGGACCAGCTGTGCTCTGCTCTCTGCTCCATGGTAC | | 78 | 378 | |
| | CATGGCCGGC | | | | |
| 73 | AGTGTGGACCAGCTGTGCTCTGCTCTCTGCTCCATGGTACCATGG | 16 | 21223 | 2122 | TSC2 |
| | CCGGCCTGGGGTTGGGGTGGGGGACCCAGTAGGGTTTTTCCCCA | | 28 | 428 | |
| | AAAGACTGCGA | | | | |
| 74 | GGCTACCCCGTGACCTGGCCGCTGGGGAGAGGTTTCATGCCTGG | 16 | 21227 | 2122 | TSC2 |
| | ATTTGGTCATCAGCTTTCAGGCCCAAAGACACTGGAGCGGCTCC | | 92 | 892 | |
| | GAGGCGCCCCAG | | | | |
| 75 | TCATCAGCTTTCAGGCCCAAAGACACTGGAGCGGCTCCGAGGCG | 16 | 21228 | 2122 | TSC2 |
| | CCCCAGAAGGCTTCTCCAGAACTGACTTGCACCTGGCCGTGGTT | | 42 | 942 | |
| | CCAGTGCTGACA | | | | |
| 76 | AAGGCTTCTCCAGAACTGACTTGCACCTGGCCGTGGTTCCAGTG | 16 | 21228 | 2122 | TSC2 |
| | CTGACAGCATTAATCTCTTACCATAACTACCTGGACAAAACCAA | | 92 | 992 | |
| | ACAGGTAGGAGG | | | | |
| 77 | GCATTAATCTCTTACCATAACTACCTGGACAAAACCAAACAGGT | 16 | 21229 | 2123 | TSC2 |
| | AGGAGGTCAGAGCAGGACAGGCGAGCTTGATGGGGCCTGGGAT | | 42 | 042 | |
| | TCGAGGGCCTGGC | | | | |
| 78 | GCGAGGCTGCCTCTGCTGCAAGCGGGTGGGGCCTGAGGTGTCCT | 16 | 21241 | 2124 | TSC2 |
| | GTCTCCTGCAGCGCGAGATGGTCTACTGCCTGGAGCAGGGCCTC | | 45 | 245 | |
| | ATCCACCGCTGT | | | | |
| 79 | TGCAGCGCGAGATGGTCTACTGCCTGGAGCAGGGCCTCATCCAC | 16 | 21241 | 2124 | TSC2 |
| | CGCTGTGCCAGCCAGTGCGTCGTGGCCTTGTCCATCTGCAGCGT | | 95 | 295 | |
| | GGAGATGCCTGA | | | | |
| 80 | GCCAGCCAGTGCGTCGTGGCCTTGTCCATCTGCAGCGTGGAGAT | 16 | 21242 | 2124 | TSC2 |
| | GCCTGACATCATCATCAAGGCGCTGCCTGTTCTGGTGGTGAAGC | | 45 | 345 | |
| | TCACGCACATCT | | | | |
| 81 | CATCATCATCAAGGCGCTGCCTGTTCTGGTGGTGAAGCTCACGC | 16 | 21242 | 2124 | TSC2 |
| | ACATCTCAGCCACAGCCAGCATGGCCGTCCCACTGCTGGAGTTC | | 95 | 395 | |
| | CTGTCCAGTGAG | | | | |
| 82 | CAGCCACAGCCAGCATGGCCGTCCCACTGCTGGAGTTCCTGTCC | 16 | 21243 | 2124 | TSC2 |
| | AGTGAGTCCCCGCCCTGCCTGCGCATGCACCCGAGAGGTTCGGG | | 45 | 445 | |
| | CTGTGTAACCTG | | | | |
| 83 | AGAGGCGCTGCACGGGACCCCGGCTCCCCTGACCACCCTCTCCA | 16 | 21257 | 2125 | TSC2 |
| | TTACCGCAGCTCTGGCCAGGCTGCCGCACCTCTACAGGAACTTT | | 46 | 846 | |
| | GCCGCGGAGCAG | | | | |
| 84 | CAGCTCTGGCCAGGCTGCCGCACCTCTACAGGAACTTTGCCGCG | 16 | 21257 | 2125 | TSC2 |
| | GAGCAGTATGCCAGTGTGTTCGCCATCTCCCTGCCGTACACCAA | | 96 | 896 | |
| | CCCCTCCAAGTG | | | | |
| 85 | TATGCCAGTGTGTTCGCCATCTCCCTGCCGTACACCAACCCCTCC | 16 | 21258 | 2125 | TSC2 |
| | AAGTGAGTGGTCGCCCCAGGCCCTGTGCCTCCCAGCCGTGGCCC | | 46 | 946 | |
| | CCGCTAGGCCT | | | | |
| 86 | GTTTTTTGCACTTCATGCCCTGGGGATGTTTCCCTGCTGCCAGGA | 16 | 21259 | 2126 | TSC2 |
| | TGGAGTGCCAGCCCCCTTCTCATCTCAGGTTTAATCAGTACATCG | | 95 | 095 | |
| | TGTGTCTGGC | | | | |
| 87 | TGCCAGCCCCCTTCTCATCTCAGGTTTAATCAGTACATCGTGTGT | 16 | 21260 | 2126 | TSC2 |
| | CTGGCCCATCACGTCATAGCCATGTGGTTCATCAGGTGCCGCCT | | 45 | 145 | |
| | GCCCTTCCGGA | | | | |
| 88 | CCATCACGTCATAGCCATGTGGTTCATCAGGTGCCGCCTGCCCTT | 16 | 21260 | 2126 | TSC2 |
| | CCGGAAGGATTTTGTCCCTTTCATCACTAAGGTGGGCTCAGGGC | | 95 | 195 | |
| | CGGTGAAGGCT | | | | |
| 89 | AGGATTTTGTCCCTTTCATCACTAAGGTGGGCTCAGGGCCGGTG | 16 | 21261 | 2126 | TSC2 |
| | AAGGCTGTGTCTCTCGGTAGGCCAGGGCTTGCTTTGCCCTTGGCT | | 45 | 245 | |
| | GTCCATGGTCG | | | | |
| 90 | CCTCCAGCCCCCATTGCCACCCCTCACTGTCTGGGTGTGCTCACT | 16 | 21264 | 2126 | TSC2 |
| | CTGCCAGGGCCTGCGGTCCAATGTCCTCTTGTCTTTTGATGACAC | | 39 | 539 | |
| | CCCCGAGAAG | | | | |
| 91 | AGGGCCTGCGGTCCAATGTCCTCTTGTCTTTTGATGACACCCCCG | 16 | 21264 | 2126 | TSC2 |
| | AGAAGGACAGCTTCAGGGCCCGGAGTACTAGTCTCAACGAGAG | | 89 | 589 | |
| | ACCCAAGAGGTA | | | | |
| 92 | GACAGCTTCAGGGCCCGGAGTACTAGTCTCAACGAGAGACCCA | 16 | 21265 | 2126 | TSC2 |
| | AGAGGTACGGCCTGCGGGGGTGTGCCTGGAGTCGGTGTGGGGT | | 39 | 639 | |
| | GGGGAAGGACATGG | | | | |
| 93 | TTCTCCCCTTCCCGGGAGCTGGGCTCTCTGGGGCGTTGGGGCTCC | 16 | 21275 | 2127 | TSC2 |
| | TTCCTCACCCGATAGTCTGAGGATAGCCAGACCCCCCAAACAAG | | 38 | 638 | |
| | GCTTGAATAAC | | | | |
| 94 | CACCCGATAGTCTGAGGATAGCCAGACCCCCCAAACAAGGCTTG | 16 | 21275 | 2127 | TSC2 |
| | AATAACTCTCCACCCGTGAAAGAATTCAAGGAGAGCTCTGCAGC | | 88 | 688 | |
| | CGAGGCCTTCCG | | | | |
| 95 | TCTCCACCCGTGAAAGAATTCAAGGAGAGCTCTGCAGCCGAGGC | 16 | 21276 | 2127 | TSC2 |
| | CTTCCGGTGCCGCAGCATCAGTGTGTCTGAACATGTGGTCCGCA | | 38 | 738 | |
| | GGTAGCGGGACT | | | | |
| 96 | GTGCCGCAGCATCAGTGTGTCTGAACATGTGGTCCGCAGGTAGC | 16 | 21276 | 2127 | TSC2 |
| | GGGACTGTCGGGTGGGGGGCACGGACCCTGGAGCTTGGCCCCGT | | 88 | 788 | |
| | GAGCACCTGGGT | | | | |
| 97 | CTTGGTGATAGGTGGCTCGGCCCGCCCTACCTGGCACCCTGACC | 16 | 21289 | 2129 | TSC2 |
| | CTGGTCACGGCCTCTCCCTCCAGCAGGATACAGACGTCCCTCAC | | 65 | 065 | |
| | CAGTGCCAGCTT | | | | |
| 98 | ACGGCCTCTCCCTCCAGCAGGATACAGACGTCCCTCACCAGTGC | 16 | 21290 | 2129 | TSC2 |
| | CAGCTTGGGGTCTGCAGATGAGAACTCCGTGGCCCAGGCTGACG | | 15 | 115 | |
| | ATAGCCTGAAAA | | | | |
| 99 | GGGGTCTGCAGATGAGAACTCCGTGGCCCAGGCTGACGATAGCC | 16 | 21290 | 2129 | TSC2 |
| | TGAAAAACCTCCACCTGGAGCTCACGGAAACCTGTCTGGACATG | | 65 | 165 | |
| | ATGGCTCGATAC | | | | |
| 100 | ACCTCCACCTGGAGCTCACGGAAACCTGTCTGGACATGATGGCT | 16 | 21291 | 2129 | TSC2 |
| | CGATACGTCTTCTCCAACTTCACGGCTGTCCCGAAGAGGTCCAG | | 15 | 215 | |
| | GCGGCACTACAG | | | | |
| 101 | GTCTTCTCCAACTTCACGGCTGTCCCGAAGAGGTCCAGGCGGCA | 16 | 21291 | 2129 | TSC2 |
| | CTACAGGGCTGGGCGGGCCTGCGGGAGCTCCACGGGCAAGCTG | | 65 | 265 | |
| | GGTTTCACGCTCC | | | | |
| 102 | GCGGCACTACAGGGCTGGGCGGGCCTGCGGGAGCTCCACGGGC | 16 | 21292 | 2129 | TSC2 |
| | AAGCTGGGTTTCACGCTCCCTGTCTTCTAGGTCTCCTGTGGGCGA | | 03 | 303 | |
| | GTTCCTCCTAGC | | | | |
| 103 | GTTTCACGCTCCCTGTCTTCTAGGTCTCCTGTGGGCGAGTTCCTC | 16 | 21292 | 2129 | TSC2 |
| | CTAGCGGGTGGCAGGACCAAAACCTGGCTGGTTGGGAACAAGC | | 53 | 353 | |
| | TTGTCACTGTGA | | | | |
| 104 | GGGTGGCAGGACCAAAACCTGGCTGGTTGGGAACAAGCTTGTC | 16 | 21293 | 2129 | TSC2 |
| | ACTGTGACGACAAGCGTGGGAACCGGGACCCGGTCGTTACTAG | | 03 | 403 | |
| | GCCTGGACTCGGGG | | | | |
| 105 | CGACAAGCGTGGGAACCGGGACCCGGTCGTTACTAGGCCTGGA | 16 | 21293 | 2129 | TSC2 |
| | CTCGGGGGAGCTGCAGTCCGGCCCGGAGTCGAGGTGACTGCACC | | 53 | 453 | |
| | TTCCTTTCCTCCG | | | | |
| 106 | GAGCTGCAGTCCGGCCCGGAGTCGAGGTGACTGCACCTTCCTTT | 16 | 21294 | 2129 | TSC2 |
| | CCTCCGCGCCTGCCAGCCTCGACACCGGCTGTCCCGAGCCCAGG | | 03 | 503 | |
| | CCCACGTGGCAC | | | | |
| 107 | GGCCCACGTGGCACCCTCGTACCAGCCTGGGGACTAAGTCCACC | 16 | 21294 | 2129 | TSC2 |
| | CTGTGCGTGGGATTCTCTTCTCAGCTCCAGCCCCGGGGTGCATGT | | 89 | 589 | |
| | GAGACAGACCA | | | | |
| 108 | GTGGGATTCTCTTCTCAGCTCCAGCCCCGGGGTGCATGTGAGAC | 16 | 21295 | 2129 | TSC2 |
| | AGACCAAGGAGGCGCCGGCCAAGCTGGAGTCCCAGGCTGGGCA | | 39 | 639 | |
| | GCAGGTGTCCCGT | | | | |
| 109 | AGGAGGCGCCGGCCAAGCTGGAGTCCCAGGCTGGGCAGCAGGT | 16 | 21295 | 2129 | TSC2 |
| | GTCCCGTGGGGCCCGGGATCGGGTCCGTTCCATGTCGGGTGAGC | | 89 | 689 | |
| | CTTGGCCCCAGCC | | | | |
| 110 | GGGGCCCGGGATCGGGTCCGTTCCATGTCGGGTGAGCCTTGGCC | 16 | 21296 | 2129 | TSC2 |
| | CCAGCCACCTCCACACAGGCACCGGGGCTCCCTCAGTTGCTGCT | | 39 | 739 | |
| | GGTCCCAGTGTT | | | | |
| 111 | TTCAGCTTGAGGCTGGTGGTTTTGCATCAGGTAAGTGGTGGTCA | 16 | 21300 | 2130 | TSC2 |
| | CCAGTCCTCTGCCCTCTTCTTCAGGGGGCCATGGTCTTCGAGTTG | | 97 | 197 | |
| | GCGCCCTGGAC | | | | |
| 112 | CTCTGCCCTCTTCTTCAGGGGGCCATGGTCTTCGAGTTGGCGCCC | 16 | 21301 | 2130 | TSC2 |
| | TGGACGTGCCGGCCTCCCAGTTCCTGGGCAGTGCCACTTCTCCA | | 47 | 247 | |
| | GGACCACGGAC | | | | |
| 113 | GTGCCGGCCTCCCAGTTCCTGGGCAGTGCCACTTCTCCAGGACC | 16 | 21301 | 2130 | TSC2 |
| | ACGGACTGCACCAGCCGCGAAACCTGAGAAGGCCTCAGCTGGC | | 97 | 297 | |
| | ACCCGGGTTCCTG | | | | |
| 114 | TGCACCAGCCGCGAAACCTGAGAAGGCCTCAGCTGGCACCCGG | 16 | 21302 | 2130 | TSC2 |
| | GTTCCTGTGCAGGAGAAGACGAACCTGGCGGCCTATGTGCCCCT | | 47 | 347 | |
| | GCTGACCCAGGGC | | | | |
| 115 | TGCAGGAGAAGACGAACCTGGCGGCCTATGTGCCCCTGCTGACC | 16 | 21302 | 2130 | TSC2 |
| | CAGGGCTGGGCGGAGATCCTGGTCCGGAGGCCCACAGGTACTG | | 97 | 397 | |
| | GGCGGGGCTGGCC | | | | |
| 116 | TGGGCGGAGATCCTGGTCCGGAGGCCCACAGGTACTGGGCGGG | 16 | 21303 | 2130 | TSC2 |
| | GCTGGCCTGAGCGCCATCTTTCTGCCAGTCACCCACAGAGCTGT | | 47 | 447 | |
| | GGACACTCAGGGG | | | | |
| 117 | AGGCCCCTGGGGGGCCAGAGATGGGTAAGGGGAGGTACTGGCC | 16 | 21315 | 2131 | TSC2 |
| | TCAGGCCAAAGGTGCTGCCGCCTCCGCAGGGAACACCAGCTGGC | | 23 | 623 | |
| | TGATGAGCCTGGA | | | | |
| 118 | AAAGGTGCTGCCGCCTCCGCAGGGAACACCAGCTGGCTGATGA | 16 | 21315 | 2131 | TSC2 |
| | GCCTGGAGAACCCGCTCAGCCCTTTCTCCTCGGACATCAACAAC | | 73 | 673 | |
| | ATGCCCCTGCAGG | | | | |
| 119 | GAACCCGCTCAGCCCTTTCTCCTCGGACATCAACAACATGCCCC | 16 | 21316 | 2131 | TSC2 |
| | TGCAGGAGCTGTCTAACGCCCTCATGGCGGCTGAGCGCTTCAAG | | 23 | 723 | |
| | GAGCACCGGGAC | | | | |
| 120 | AGCTGTCTAACGCCCTCATGGCGGCTGAGCGCTTCAAGGAGCAC | 16 | 21316 | 2131 | TSC2 |
| | CGGGACACAGCCCTGTACAAGTCACTGTCGGTGCCGGCAGCCAG | | 73 | 773 | |
| | CACGGCCAAACC | | | | |
| 121 | ACAGCCCTGTACAAGTCACTGTCGGTGCCGGCAGCCAGCACGGC | 16 | 21317 | 2131 | TSC2 |
| | CAAACCCCCTCCTCTGCCTCGCTCCAACACAGGTGAGTGGCATG | | 23 | 823 | |
| | GCGGGCCTTGGC | | | | |
| 122 | CCCTCCTCTGCCTCGCTCCAACACAGGTGAGTGGCATGGCGGGC | 16 | 21317 | 2131 | TSC2 |
| | CTTGGCACGGGCTCTGCTCCCACTGGCCTGGTGCTCCCGGTGAC | | 73 | 873 | |
| | GGCAATGTGGCT | | | | |
| 123 | CTCTGCTCGACCTGTGTGTAGCCCCTCCTCCTGCTGACGTGGCCG | 16 | 21323 | 2132 | TSC2 |
| | CACACGGCCTTCCCTTGCAGTGGCCTCTTTCTCCTCCCTGTACCA | | 71 | 471 | |
| | GTCCAGCTGC | | | | |
| 124 | GGCCTTCCCTTGCAGTGGCCTCTTTCTCCTCCCTGTACCAGTCCA | 16 | 21324 | 2132 | TSC2 |
| | GCTGCCAAGGACAGCTGCACAGGAGCGTTTCCTGGGCAGGTATC | | 21 | 521 | |
| | GCCTCTCAGAG | | | | |
| 125 | CAAGGACAGCTGCACAGGAGCGTTTCCTGGGCAGGTATCGCCTC | 16 | 21324 | 2132 | TSC2 |
| | TCAGAGGGAAGCGGTTGGCTGCAGAGCGCCACTCTGCCTCATAG | | 71 | 571 | |
| | GTGCTGTGCTCG | | | | |
| 126 | GGCCACGTCAGGGCCAGGGCCTGGCCCAGCCCCACATCCAGCA | 16 | 21336 | 2133 | TSC2 |
| | GCCCCGTCTGTGTCCTCCCAGACTCCGCCGTGGTCATGGAGGAG | | 31 | 731 | |
| | GGAAGTCCGGGCG | | | | |
| 127 | CTGTGTCCTCCCAGACTCCGCCGTGGTCATGGAGGAGGGAAGTC | 16 | 21336 | 2133 | TSC2 |
| | CGGGCGAGGTTCCTGTGCTGGTGGAGCCCCCAGGGTTGGAGGAC | | 81 | 781 | |
| | GTTGAGGCAGCG | | | | |
| 128 | AGGTTCCTGTGCTGGTGGAGCCCCCAGGGTTGGAGGACGTTGAG | 16 | 21337 | 2133 | TSC2 |
| | GCAGCGCTAGGCATGGACAGGCGCACGGATGCCTACAGCAGGG | | 31 | 831 | |
| | TGAGTGTGGCTCA | | | | |
| 129 | CTAGGCATGGACAGGCGCACGGATGCCTACAGCAGGGTGAGTG | 16 | 21337 | 2133 | TSC2 |
| | TGGCTCAGAGCCTGGACCCTGCTGACCTCGGGGGGCTCCTTAGG | | 81 | 881 | |
| | GGAGGCAGGGCTC | | | | |
| 130 | GGTGGGCTCGAGGGTGCCTGCTGACAGGGGTTCTCTTTGGGATG | 16 | 21341 | 2134 | TSC2 |
| | GTCCTTTCTAGTCGTCCTCAGTCTCCAGCCAGGAGGAGAAGTCG | | 73 | 273 | |
| | CTCCACGCGGAG | | | | |
| 131 | TCTAGTCGTCCTCAGTCTCCAGCCAGGAGGAGAAGTCGCTCCAC | 16 | 21342 | 2134 | TSC2 |
| | GCGGAGGAGCTGGTTGGCAGGGGCATCCCCATCGAGCGAGTCG | | 23 | 323 | |
| | TCTCCTCGGAGGG | | | | |
| 132 | GAGCTGGTTGGCAGGGGCATCCCCATCGAGCGAGTCGTCTCCTC | 16 | 21342 | 2134 | TSC2 |
| | GGAGGGTGGCCGGCCCTCTGTGGACCTCTCCTTCCAGCCCTCGC | | 73 | 373 | |
| | AGCCCCTGAGCA | | | | |
| 133 | TGGCCGGCCCTCTGTGGACCTCTCCTTCCAGCCCTCGCAGCCCCT | 16 | 21343 | 2134 | TSC2 |
| | GAGCAAGTCCAGCTCCTCTCCCGAGCTGCAGACTCTGCAGGACA | | 23 | 423 | |
| | TCCTCGGGGAC | | | | |
| 134 | AGTCCAGCTCCTCTCCCGAGCTGCAGACTCTGCAGGACATCCTC | 16 | 21343 | 2134 | TSC2 |
| | GGGGACCCTGGGGACAAGGCCGACGTGGGCCGGCTGAGCCCTG | | 73 | 473 | |
| | AGGTTAAGGCCCG | | | | |
| 135 | CCTGGGGACAAGGCCGACGTGGGCCGGCTGAGCCCTGAGGTTA | 16 | 21344 | 2134 | TSC2 |
| | AGGCCCGGTCACAGTCAGGGACCCTGGACGGGGAAAGTGCTGC | | 23 | 523 | |
| | CTGGTCGGCCTCGG | | | | |
| 136 | GTCACAGTCAGGGACCCTGGACGGGGAAAGTGCTGCCTGGTCG | 16 | 21344 | 2134 | TSC2 |
| | GCCTCGGGCGAAGACAGTCGGGGCCAGCCCGAGGGTCCCTTGCC | | 73 | 573 | |
| | TTCCAGCTCCCCC | | | | |
| 137 | GCGAAGACAGTCGGGGCCAGCCCGAGGGTCCCTTGCCTTCCAGC | 16 | 21345 | 2134 | TSC2 |
| | TCCCCCCGCTCGCCCAGTGGCCTCCGGCCCCGAGGTTACACCAT | | 23 | 623 | |
| | CTCCGACTCGGC | | | | |
| 138 | CGCTCGCCCAGTGGCCTCCGGCCCCGAGGTTACACCATCTCCGA | 16 | 21345 | 2134 | TSC2 |
| | CTCGGCCCCATCACGCAGGGGCAAGAGAGTAGAGAGGGACGCC | | 73 | 673 | |
| | TTAAAGAGCAGAG | | | | |
| 139 | CCCATCACGCAGGGGCAAGAGAGTAGAGAGGGACGCCTTAAAG | 16 | 21346 | 2134 | TSC2 |
| | AGCAGAGCCACAGCCTCCAATGCAGAGAAAGTGCCAGGCATCA | | 23 | 723 | |
| | ACCCCAGGTGGGCC | | | | |
| 140 | CCACAGCCTCCAATGCAGAGAAAGTGCCAGGCATCAACCCCAG | 16 | 21346 | 2134 | TSC2 |
| | GTGGGCCTCTTGCTTCCGGGCGGGGCTCCTGACACCTCTCCTGCG | | 73 | 773 | |
| | GGAACCTGGTGC | | | | |
| 141 | TGGGCTGTGGCTGCCCTGGCCAGGCCCTCACCTGGGTGCCCACC | 16 | 21348 | 2134 | TSC2 |
| | ATCCCCTCCCTGTGCAGTTTCGTGTTCCTGCAGCTCTACCATTCC | | 90 | 990 | |
| | CCCTTCTTTGG | | | | |
| 142 | TCCCTGTGCAGTTTCGTGTTCCTGCAGCTCTACCATTCCCCCTTCT | 16 | 21349 | 2135 | TSC2 |
| | TTGGCGACGAGTCAAACAAGCCAATCCTGCTGCCCAATGAGGTA | | 40 | 040 | |
| | GGCGTGGCCT | | | | |
| 143 | CGACGAGTCAAACAAGCCAATCCTGCTGCCCAATGAGGTAGGC | 16 | 21349 | 2135 | TSC2 |
| | GTGGCCTCCCTCTCCTGCATCCGCTGGAGCTGTGTGGCTCGGGTG | | 90 | 090 | |
| | AATGGTGGGGGG | | | | |
| 144 | CGGCCTCCTGTGGACGGGCGTCTGGGGCTCAGGCAGGGCTCTGT | 16 | 21351 | 2135 | TSC2 |
| | GTGCCACAGTCACAGTCCTTTGAGCGGTCGGTGCAGCTCCTCGA | | 77 | 277 | |
| | CCAGATCCCATC | | | | |
| 145 | CAGTCACAGTCCTTTGAGCGGTCGGTGCAGCTCCTCGACCAGAT | 16 | 21352 | 2135 | TSC2 |
| | CCCATCATACGACACCCACAAGATCGCCGTCCTGTATGTTGGAG | | 27 | 327 | |
| | AAGGCCAGGTGA | | | | |
| 146 | ATACGACACCCACAAGATCGCCGTCCTGTATGTTGGAGAAGGCC | 16 | 21352 | 2135 | TSC2 |
| | AGGTGAGGCTGCGGGGCCGGCCTAGGTGCCTGGACAGGGCCAG | | 77 | 377 | |
| | CTGGGCCTCAGCC | | | | |
| 147 | CGGGGCAGGGCCCGGCCCGGGAGTGATGCCACCCTGCCTCTCCC | 16 | 21361 | 2136 | TSC2 |
| | CTCTCCCCACAGAGCAACAGCGAGCTCGCCATCCTGTCCAATGA | | 37 | 237 | |
| | GCATGGCTCCTA | | | | |
| 148 | CCACAGAGCAACAGCGAGCTCGCCATCCTGTCCAATGAGCATGG | 16 | 21361 | 2136 | TSC2 |
| | CTCCTACAGGTACACGGAGTTCCTGACGGGCCTGGGCCGGCTCA | | 87 | 287 | |
| | TCGAGCTGAAGG | | | | |
| 149 | CAGGTACACGGAGTTCCTGACGGGCCTGGGCCGGCTCATCGAGC | 16 | 21362 | 2136 | TSC2 |
| | TGAAGGACTGCCAGCCGGACAAGGTGTACCTGGGAGGCCTGGA | | 37 | 337 | |
| | CGTGTGTGGTGAG | | | | |
| 150 | ACTGCCAGCCGGACAAGGTGTACCTGGGAGGCCTGGACGTGTGT | 16 | 21362 | 2136 | TSC2 |
| | GGTGAGGACGGCCAGTTCACCTACTGCTGGCACGATGACATCAT | | 87 | 387 | |
| | GCAAGGTACGGC | | | | |
| 151 | GACGGCCAGTTCACCTACTGCTGGCACGATGACATCATGCAAGG | 16 | 21363 | 2136 | TSC2 |
| | TACGGCCTGGCGCCTACCCGCTCCTGCTGCCCCAGGCCTCAGGG | | 37 | 437 | |
| | CACGGCTCCCAT | | | | |
| 152 | ACAGAGGGCCTCAGCACTGGCCCCACAAACCCATCCGGCCCTGC | 16 | 21366 | 2136 | TSC2 |
| | TCACCCTCAGCCGTCTTCCACATCGCCACCCTGATGCCCACCAA | | 7 8 | 778 | |
| | GGACGTGGACAA | | | | |
| 153 | TCAGCCGTCTTCCACATCGCCACCCTGATGCCCACCAAGGACGT | 16 | 21367 | 2136 | TSC2 |
| | GGACAAGCACCGCTGCGACAAGAAGCGCCACCTGGGCAACGAC | | 28 | 828 | |
| | TTTGTGTCCATTG | | | | |
| 154 | GCACCGCTGCGACAAGAAGCGCCACCTGGGCAACGACTTTGTGT | 16 | 21367 | 2136 | TSC2 |
| | CCATTGTCTACAATGACTCCGGTGAGGACTTCAAGCTTGGCACC | | 78 | 878 | |
| | ATCAAGGTGAGT | | | | |
| 155 | TCTACAATGACTCCGGTGAGGACTTCAAGCTTGGCACCATCAAG | 16 | 21368 | 2136 | TSC2 |
| | GTGAGTGAGGGGCCGTCAGTGAGGCTGGGCCCCAGGCAGGTGC | | 28 | 928 | |
| | CCACTGCTGTGTC | | | | |
| 156 | CCGAGATCAGCCTTCAGCACACGCTGTGTGCGGGGATGACCCTT | 16 | 21378 | 2137 | TSC2 |
| | TCTCTTGTCCGGGCAGGGCCAGTTCAACTTTGTCCACGTGATCGT | | 03 | 903 | |
| | CACCCCGCTGG | | | | |
| 157 | GTCCGGGCAGGGCCAGTTCAACTTTGTCCACGTGATCGTCACCC | 16 | 21378 | 2137 | TSC2 |
| | CGCTGGACTACGAGTGCAACCTGGTGTCCCTGCAGTGCAGGAAA | | 53 | 953 | |
| | GGTAGGGCCGGG | | | | |
| 158 | ACTACGAGTGCAACCTGGTGTCCCTGCAGTGCAGGAAAGGTAGG | 16 | 21379 | 2138 | TSC2 |
| | GCCGGGTGGGGCCCTGCAGTGCAGGAAAGGTAGGGCCGGGTGG | | 03 | 003 | |
| | GGCCCTGCAGTGT | | | | |
| 159 | TGCAGTGTGGCGCCAAGAGCCCTGGGCCTGGCGTGACCACCAAG | 16 | 21379 | 2138 | TSC2 |
| | TCTCCCCAGACATGGAGGGCCTTGTGGACACCAGCGTGGCCAAG | | 95 | 095 | |
| | ATCGTGTCTGAC | | | | |
| 160 | CAGACATGGAGGGCCTTGTGGACACCAGCGTGGCCAAGATCGT | 16 | 21380 | 2138 | TSC2 |
| | GTCTGACCGCAACCTGCCCTTCGTGGCCCGCCAGATGGCCCTGC | | 45 | 145 | |
| | ACGCAAATGTGAG | | | | |
| 161 | CGCAACCTGCCCTTCGTGGCCCGCCAGATGGCCCTGCACGCAAA | 16 | 21380 | 2138 | TSC2 |
| | TGTGAGTGGGGGTGGGTCCAGGCGTGAGCTGGTGGGACAGGCC | | 95 | 195 | |
| | CAGGTGCCACCTG | | | | |
| 162 | AGGCCCAGGTGCCACCTGATAGTGAGCTCACCCCCTGCCTACGT | 16 | 21381 | 2138 | TSC2 |
| | CCCCAGATGGCCTCACAGGTGCATCATAGCCGCTCCAACCCCAC | | 77 | 277 | |
| | CGATATCTACCC | | | | |
| 163 | ATGGCCTCACAGGTGCATCATAGCCGCTCCAACCCCACCGATAT | 16 | 21382 | 2138 | TSC2 |
| | CTACCCCTCCAAGTGGATTGCCCGGCTCCGCCACATCAAGCGGC | | 27 | 327 | |
| | TCCGCCAGCGGG | | | | |
| 164 | CTCCAAGTGGATTGCCCGGCTCCGCCACATCAAGCGGCTCCGCC | 16 | 21382 | 2138 | TSC2 |
| | AGCGGGTAGGGAATATGGGGCTCCCTCAGCGGGGTGTGCTGGCT | | 77 | 377 | |
| | GCCCAAGCTGTG | | | | |
| 165 | GCGGGTGTGTGGGCAGAGCGGTTGCCACGCCTCCCAGACTTACT | 16 | 21383 | 2138 | TSC2 |
| | GCCCAAGCCGCCTCTGCCTTCAGATCTGCGAGGAAGCCGCCTAC | | 79 | 479 | |
| | TCCAACCCCAGC | | | | |
| 166 | GCCGCCTCTGCCTTCAGATCTGCGAGGAAGCCGCCTACTCCAAC | 16 | 21384 | 2138 | TSC2 |
| | CCCAGCCTACCTCTGGTGCACCCTCCGTCCCATAGCAAAGCCCC | | 29 | 529 | |
| | TGCACAGACTCC | | | | |
| 167 | CTACCTCTGGTGCACCCTCCGTCCCATAGCAAAGCCCCTGCACA | 16 | 21384 | 2138 | TSC2 |
| | GACTCCAGCCGAGCCCACACCTGGCTATGAGGTGGGCCAGCGG | | 79 | 579 | |
| | AAGCGCCTCATCT | | | | |
| 168 | AGCCGAGCCCACACCTGGCTATGAGGTGGGCCAGCGGAAGCGC | 16 | 21385 | 2138 | TSC2 |
| | CTCATCTCCTCGGTGGAGGACTTCACCGAGTTTGTGTGAGGCCG | | 29 | 629 | |
| | GGGCCCTCCCTCC | | | | |
| 169 | CCTCGGTGGAGGACTTCACCGAGTTTGTGTGAGGCCGGGGCCCT | 16 | 21385 | 2138 | TSC2 |
| | CCCTCCTGCACTGGCCTTGGACGGTATTGCCTGTCAGTGAAATA | | 79 | 679 | |
| | AATAAAGTCCTG | | | | |
| 170 | GTGCATTCACACCTCCTGTTCTGTGCCAACAATATGCAAGTTAAC | 9 | 13577 | 1357 | TSC1 |
| | ACTGATTGACCATCATTCCTTAGCTGTGTTCATGATGAGTCTCAT | | 1557 | 7165 | |
| | TGTAGTCCAT | | | 7 | |
| 171 | TTGACCATCATTCCTTAGCTGTGTTCATGATGAGTCTCATTGTAG | 9 | 13577 | 1357 | TSC1 |
| | TCCATGATATGTAGCTGTCCAACACTGTCCGGGGTCGGGGGAGA | | 1607 | 7170 | |
| | CGGGTGAGGGC | | | 7 | |
| 172 | GATATGTAGCTGTCCAACACTGTCCGGGGTCGGGGGAGACGGGT | 9 | 13577 | 1357 | TSC1 |
| | GAGGGCCATCTAGGTTCAGGGGAATCTTGGCTTCCACACCCAAG | | 1657 | 7175 | |
| | TCTTTGCCCAGT | | | 7 | |
| 173 | CATCTAGGTTCAGGGGAATCTTGGCTTCCACACCCAAGTCTTTGC | 9 | 13577 | 1357 | TSC1 |
| | CCAGTTCTGTCTTTAGGCTCTCAGAAAGGCTACTGGTCATGCCGT | | 1707 | 7180 | |
| | CCTCATCACA | | | 7 | |
| 174 | TCTGTCTTTAGGCTCTCAGAAAGGCTACTGGTCATGCCGTCCTCA | 9 | 13577 | 1357 | TSC1 |
| | TCACACTGGCTCTCGCTCTTATTACGAAATAACTCTCGAGCCTTC | | 1757 | 7185 | |
| | ATACCCAGGA | | | 7 | |
| 175 | CTGGCTCTCGCTCTTATTACGAAATAACTCTCGAGCCTTCATACC | 9 | 13577 | 1357 | TSC1 |
| | CAGGAAGCTTTTTGAACTGGGAAGTGAGCCCACAGTGGTGGGG | | 1807 | 7190 | |
| | ATGCTGGCAGAC | | | 7 | |
| 176 | AGCTTTTTGAACTGGGAAGTGAGCCCACAGTGGTGGGGATGCTG | 9 | 13577 | 1357 | TSC1 |
| | GCAGACGCTTCTCCCATAGTCGTCTCCCACCGACTGCTGAATGG | | 1857 | 7195 | |
| | GCCTGCCCTCTG | | | 7 | |
| 177 | GCTTCTCCCATAGTCGTCTCCCACCGACTGCTGAATGGGCCTGCC | 9 | 13577 | 1357 | TSC1 |
| | CTCTGGTGTGGGGGTTTCTCTGGGGTAGAAAGCTCGCTGCTGCT | | 1907 | 7200 | |
| | GCTGCTGCTGC | | | 7 | |
| 178 | GTGTGGGGGTTTCTCTGGGGTAGAAAGCTCGCTGCTGCTGCTGC | 9 | 13577 | 1357 | TSC1 |
| | TGCTGCCTCCACCACCTCTGCTTCCACTACTGCCCCGGGCGCTGC | | 1957 | 7205 | |
| | TGGGCCTGGGG | | | 7 | |
| 179 | CTCCACCACCTCTGCTTCCACTACTGCCCCGGGCGCTGCTGGGCC | 9 | 13577 | 1357 | TSC1 |
| | TGGGGGTCTTGGTCTCACCGTTGTGGCCAGATGCCTCTTCATTGT | | 2007 | 7210 | |
| | GCCCTACCAT | | | 7 | |
| 180 | GTCTTGGTCTCACCGTTGTGGCCAGATGCCTCTTCATTGTGCCCT | 9 | 13577 | 1357 | TSC1 |
| | ACCATGGAATCTGAGCACCCGTCATTACAACAGTCAAGCCTGTA | | 2057 | 7215 | |
| | AGAAAGCCGGG | | | 7 | |
| 181 | GGAATCTGAGCACCCGTCATTACAACAGTCAAGCCTGTAAGAAA | 9 | 13577 | 1357 | TSC1 |
| | GCCGGGGAGGAAAAAAGGAGCTGGTGATTGGACTGTCCACATT | | 2107 | 7220 | |
| | CGGAGGATGTGGA | | | 7 | |
| 182 | CGTGACACAGTCCTTATGCTGGAATTGGCAGCTTAGTCCCAAGG | 9 | 13577 | 1357 | TSC1 |
| | TCATGAATCAGTTCTTTGTTCCTACCTTTCTTCTGCTGCTTCAGCT | | 2501 | 7260 | |
| | GCTTCTGCTT | | | 1 | |
| 183 | ATCAGTTCTTTGTTCCTACCTTTCTTCTGCTGCTTCAGCTGCTTCT | 9 | 13577 | 1357 | TSC1 |
| | GCTTTTTCTTCTTCAAGTTTTTTCAGGAGGCCATCTTTCTCCAACC | | 2551 | 7265 | |
| | TGCCATAT | | | 1 | |
| 184 | TTTCTTCTTCAAGTTTTTTCAGGAGGCCATCTTTCTCCAACCTGCC | 9 | 13577 | 1357 | TSC1 |
| | ATATAAATCTAAGATCTCCAATTCAAACACCTGGGTTATCCTTTT | | 2601 | 7270 | |
| | CTGAGCCTC | | | 1 | |
| 185 | AAATCTAAGATCTCCAATTCAAACACCTGGGTTATCCTTTTCTGA | 9 | 13577 | 1357 | TSC1 |
| | GCCTCATACCTGCTCTCTGCGGCCTGCAGCTGTCCTCTGAAAGAT | | 2651 | 7275 | |
| | ACAGACCAGC | | | 1 | |
| 186 | ATACCTGCTCTCTGCGGCCTGCAGCTGTCCTCTGAAAGATACAG | 9 | 13577 | 1357 | TSC1 |
| | ACCAGCCAGAATATAGGAAGTTCCACTTAATAAAAACACAAAA | | 2701 | 7280 | |
| | GCCTTTCCTGATG | | | 1 | |
| 187 | AATATAGGAAGTTCCACTTAATAAAAACACAAAAGCCTTTCCTG | 9 | 13577 | 1357 | TSC1 |
| | ATGAAAGTTACCTTGCCTGGAGTTTGACATCCTCTAGATATTTCT | | 2754 | 7285 | |
| | TCTGTTCCAAA | | | 4 | |
| 188 | GTTACCTTGCCTGGAGTTTGACATCCTCTAGATATTTCTTCTGTT | 9 | 13577 | 1357 | TSC1 |
| | CCAAAAGAAGGTGGTCTTTCTTGGCCAGGTGAGATTCCAGTTCC | | 2804 | 7290 | |
| | AAAATCCGTTT | | | 4 | |
| 189 | AGAAGGTGGTCTTTCTTGGCCAGGTGAGATTCCAGTTCCAAAAT | 9 | 13577 | 1357 | TSC1 |
| | CCGTTTTTGGGAGGTATCAAGCCTCTGAGTCTGCTGGAGAACAT | | 2854 | 7295 | |
| | GGCTTCTGTTTT | | | 4 | |
| 190 | TTGGGAGGTATCAAGCCTCTGAGTCTGCTGGAGAACATGGCTTC | 9 | 13577 | 1357 | TSC1 |
| | TGTTTTTTTCTAGCTCTTTCCGATAGGCGGCTTTCATCATTTCTAC | | 2904 | 7300 | |
| | TTCCTGAAAA | | | 4 | |
| 191 | TTTCTAGCTCTTTCCGATAGGCGGCTTTCATCATTTCTACTTCCTG | 9 | 13577 | 1357 | TSC1 |
| | AAAAAAAAAAAAAAAAAAGACTGGAATTAGTACTTATAAAAAA | | 2954 | 7305 | |
| | TAAACATGCTG | | | 4 | |
| 192 | GACATACTGTCTGGGTCTGAAACGCTTTCCCCACTAAGGTCTGG | 9 | 13577 | 1357 | TSC1 |
| | CTCCCGAGCCCTGGCATACCTTTGTGGTATCTGAGTGCTTGTTCT | | 6038 | 7613 | |
| | GCAGTTGTTCC | | | 8 | |
| 193 | AGCCCTGGCATACCTTTGTGGTATCTGAGTGCTTGTTCTGCAGTT | 9 | 13577 | 1357 | TSC1 |
| | GTTCCAAATAGAGCTCGTTGACCTCCCCAAGAACCAACAGCTGC | | 6088 | 7618 | |
| | CTGTTCAAGAA | | | 8 | |
| 194 | AAATAGAGCTCGTTGACCTCCCCAAGAACCAACAGCTGCCTGTT | 9 | 13577 | 1357 | TSC1 |
| | CAAGAACTCCATCTGCTGCTGGACCGACTCACTGTTTGAGAGCT | | 6138 | 7623 | |
| | AACCAAAAAACA | | | 8 | |
| 195 | CTCCATCTGCTGCTGGACCGACTCACTGTTTGAGAGCTAACCAA | 9 | 13577 | 1357 | TSC1 |
| | AAAACATGAGCAAAGTGAAAAATCCGACGACATAAAACTAGCA | | 6188 | 7628 | |
| | CATAGACGTCATT | | | 8 | |
| 196 | ACCTGTCTGAAGGAAGAATGTTAGCAAATGGTGTTTCAGCAGAT | 9 | 13577 | 1357 | TSC1 |
| | TCAGGTCTGCCTCATTTCTTCTTACCTTTTGGGAAACCTGACTGA | | 6906 | 7700 | |
| | GCAGCAGCTCA | | | 6 | |
| 197 | CTGCCTCATTTCTTCTTACCTTTTGGGAAACCTGACTGAGCAGCA | 9 | 13577 | 1357 | TSC1 |
| | GCTCAGTGTGACACACCTTGTTGTTGGCCTTCTTCAGTTCTATCC | | 6956 | 7705 | |
| | GCAGCTCCGC | | | 6 | |
| 198 | GTGTGACACACCTTGTTGTTGGCCTTCTTCAGTTCTATCCGCAGC | 9 | 13577 | 1357 | TSC1 |
| | TCCGCAATCATGTTCCTGCAGTCCTCCAGCTTCGTCTGCCCAAAG | | 7006 | 7710 | |
| | AGACGTGGAC | | | 6 | |
| 199 | AATCATGTTCCTGCAGTCCTCCAGCTTCGTCTGCCCAAAGAGAC | 9 | 13577 | 1357 | TSC1 |
| | GTGGACATGAAGTTTGAGGAACACCAACAGGCCAGATCACAGG | | 7056 | 7715 | |
| | CCTACCTAGCCAC | | | 6 | |
| 200 | CTCCCCACTGCTCTCCGGCATTCTCGCAGTTGGCTTTGCCTGGTG | 9 | 13577 | 1357 | TSC1 |
| | CTGCAGTTTATACCTGTAATTCCTGGCTCTGGTTGTAGAATTCCT | | 7933 | 7803 | |
| | CTCGGTCATG | | | 3 | |
| 201 | GTTTATACCTGTAATTCCTGGCTCTGGTTGTAGAATTCCTCTCGG | 9 | 13577 | 1357 | TSC1 |
| | TCATGCTGCAGCTGTCTGATCTGGCTGTGGAGCTTGGTTACCATA | | 7983 | 7808 | |
| | GTGTCACGCT | | | 3 | |
| 202 | CTGCAGCTGTCTGATCTGGCTGTGGAGCTTGGTTACCATAGTGTC | 9 | 13577 | 1357 | TSC1 |
| | ACGCTGCTCCTGGAGCTGATTGTATCTAGCTTGTTCTTTCTGCAG | | 8033 | 7813 | |
| | ACTAACCTTC | | | 3 | |
| 203 | GCTCCTGGAGCTGATTGTATCTAGCTTGTTCTTTCTGCAGACTAA | 9 | 13577 | 1357 | TSC1 |
| | CCTTCCACATCTGGATGTCCTTCTCTTGTAACTTCAACTGATCTTT | | 8083 | 7818 | |
| | CTAGCAGAG | | | 3 | |
| 204 | CACATCTGGATGTCCTTCTCTTGTAACTTCAACTGATCTTTCTAG | 9 | 13577 | 1357 | TSC1 |
| | CAGAGACCAGAAATGTCATCATTTTAGCTGTCTTCCAACACAGG | | 8133 | 7823 | |
| | CAATTTAACAC | | | 3 | |
| 205 | AAGCTATCATGCTGACCCAAAACAAAACAAAAAGCAAGCTCCA | 9 | 13577 | 1357 | TSC1 |
| | CCTGTCCCCTCCCCAGTCCTCACCATGGCAGCATTATGTTCCTCC | | 8971 | 7907 | |
| | AGAGCTGCTGCT | | | 1 | |
| 206 | CCTCCCCAGTCCTCACCATGGCAGCATTATGTTCCTCCAGAGCTG | 9 | 13577 | 1357 | TSC1 |
| | CTGCTTTGATCACCTTGCGGAGGAGCCGCCTGTTCCGGAGGGCA | | 9021 | 7912 | |
| | TGCTGCTGCCT | | | 1 | |
| 207 | TTGATCACCTTGCGGAGGAGCCGCCTGTTCCGGAGGGCATGCTG | 9 | 13577 | 1357 | TSC1 |
| | CTGCCTCTTAAAACGCTCATAGAGTAACTGGTTGTGCAGTAAAA | | 9071 | 7917 | |
| | GCAACTGGTCTC | | | 1 | |
| 208 | CTTAAAACGCTCATAGAGTAACTGGTTGTGCAGTAAAAGCAACT | 9 | 13577 | 1357 | TSC1 |
| | GGTCTCGGAGGGTGCGGATCTCATCTGAAGGAGGAGAGCCTGAT | | 9121 | 7922 | |
| | TGTAAAGCAGAG | | | 1 | |
| 209 | GGAGGGTGCGGATCTCATCTGAAGGAGGAGAGCCTGATTGTAA | 9 | 13577 | 1357 | TSC1 |
| | AGCAGAGGGAGGGTGGCAGAAATGCCTTTTACAGATGGTTCAAT | | 9171 | 7927 | |
| | CAAGCCCCCTTCC | | | 1 | |
| 210 | AAGCAAGCAGGAACCATGTGGGCTGGATTTGGAGCTAAAGTAA | 9 | 13577 | 1357 | TSC1 |
| | CAACTTTACCTCCAAAGTGGGTCCAGTCGACAGACTTGCTGGGT | | 9745 | 7984 | |
| | AAAGGCAACCTAG | | | 5 | |
| 211 | ACCTCCAAAGTGGGTCCAGTCGACAGACTTGCTGGGTAAAGGCA | 9 | 13577 | 1357 | TSC1 |
| | ACCTAGGAAGAAAGTTTTTGAGTAACAAAGTTACCGATCTTACC | | 9795 | 7989 | |
| | AAGAAAAAAACG | | | 5 | |
| 212 | CTCTTACACTTTCTGTACTTCACAATAAAATGGACCATTTAACAC | 9 | 13578 | 1357 | TSC1 |
| | AGAAGAGAGTGCCCCAGTCCCTTACTTGTTCAGCTCCTTGCTGTG | | 0897 | 8099 | |
| | CGCGTCTGCT | | | 7 | |
| 213 | AGAGTGCCCCAGTCCCTTACTTGTTCAGCTCCTTGCTGTGCGCGT | 9 | 13578 | 1357 | TSC1 |
| | CTGCTCCCTGCTGTATCAGTCTGTCCAGCACTTCCATTGGGGAGG | | 0947 | 8104 | |
| | TAGAGGGCAC | | | 7 | |
| 214 | CCCTGCTGTATCAGTCTGTCCAGCACTTCCATTGGGGAGGTAGA | 9 | 13578 | 1357 | TSC1 |
| | GGGCACACCATCTTCCTCTGTGTTTCCTTTTGCTTTCTTTAACAGC | | 0997 | 8109 | |
| | TCCTCAGTCT | | | 7 | |
| 215 | ACCATCTTCCTCTGTGTTTCCTTTTGCTTTCTTTAACAGCTCCTCA | 9 | 13578 | 1357 | TSC1 |
| | GTCTTCCTGATGACAAAATGATGGGCTGTCTTTGGCAATGCCAC | | 1047 | 8114 | |
| | CTCAAAAAGA | | | 7 | |
| 216 | TCCTGATGACAAAATGATGGGCTGTCTTTGGCAATGCCACCTCA | 9 | 13578 | 1357 | TSC1 |
| | AAAAGATGATCATACGGGGGAGGCTGCCCGCTTCCAAAGCCCA | | 1097 | 8119 | |
| | CTCTCGTCGGAGG | | | 7 | |
| 217 | TGATCATACGGGGGAGGCTGCCCGCTTCCAAAGCCCACTCTCGT | 9 | 13578 | 1357 | TSC1 |
| | CGGAGGTGGAATTTTACAAGGACTGGGAGTGAAGATACTGGTCT | | 1147 | 8124 | |
| | CCAAAGAAGTCT | | | 7 | |
| 218 | TGGAATTTTACAAGGACTGGGAGTGAAGATACTGGTCTCCAAAG | 9 | 13578 | 1357 | TSC1 |
| | AAGTCTGGCATTCCCTGTCTCCCGCAGGGCTTTCATCAGCACTGC | | 1197 | 8129 | |
| | CGCAGGGCAGG | | | 7 | |
| 219 | GGCATTCCCTGTCTCCCGCAGGGCTTTCATCAGCACTGCCGCAG | 9 | 13578 | 1357 | TSC1 |
| | GGCAGGTCTATGGGAGTAAAGGCTTGCTTTGGTGTGTCAGGCCC | | 1247 | 8134 | |
| | AAGCTTGTCCAG | | | 7 | |
| 220 | TCTATGGGAGTAAAGGCTTGCTTTGGTGTGTCAGGCCCAAGCTT | 9 | 13578 | 1357 | TSC1 |
| | GTCCAGGGAGGAGTGTAAAGGCTCAGGGTTCACGCTGGCGCCCT | | 1297 | 8139 | |
| | GAGAACTGGAGG | | | 7 | |
| 221 | GGAGGAGTGTAAAGGCTCAGGGTTCACGCTGGCGCCCTGAGAA | 9 | 13578 | 1357 | TSC1 |
| | CTGGAGGCTGCCGAGTGGGTCTTCCGCTGAGAACCTGGGAGACT | | 1347 | 8144 | |
| | GTCTCGGTAAAAG | | | 7 | |
| 222 | CTGCCGAGTGGGTCTTCCGCTGAGAACCTGGGAGACTGTCTCGG | 9 | 13578 | 1357 | TSC1 |
| | TAAAAGGGAGAGTCAAAGCCTCCTCGAGGAACCACAGGCTCTG | | 1397 | 8149 | |
| | CCTCTGCTGTGGT | | | 7 | |
| 223 | GGAGAGTCAAAGCCTCCTCGAGGAACCACAGGCTCTGCCTCTGC | 9 | 13578 | 1357 | TSC1 |
| | TGTGGTGATCTCAGAAAGTTCTCTAGATATTGCAGCTGAGAGGA | | 1447 | 8154 | |
| | AGAGAGGAAACA | | | 7 | |
| 224 | GATCTCAGAAAGTTCTCTAGATATTGCAGCTGAGAGGAAGAGAG | 9 | 13578 | 1357 | TSC1 |
| | GAAACAAAAGAAATGGCAGTCGGTATTCCACCTGGGAAAGACT | | 1497 | 8159 | |
| | AGGCAGTTTGGGT | | | 7 | |
| 225 | GCACAAAATCCCAGATTTATAGCAGAGCGAGGGTCAGGTTTTAT | 9 | 13578 | 1357 | TSC1 |
| | CAACTCATAGCAATCCCACATACATTACCTTCTTCTTTATCTTTTT | | 2045 | 8214 | |
| | CAATACTATC | | | 5 | |
| 226 | ATAGCAATCCCACATACATTACCTTCTTCTTTATCTTTTTCAATA | 9 | 13578 | 1357 | TSC1 |
| | CTATCTTCTTCAGAGGCCAGATCACCTAAAAACCCTGGAAGATC | | 2095 | 8219 | |
| | ACTTAGAGTGA | | | 5 | |
| 227 | TTCTTCAGAGGCCAGATCACCTAAAAACCCTGGAAGATCACTTA | 9 | 13578 | 1357 | TSC1 |
| | GAGTGACAGAACCTTTGCTGCCAGGTGGCTCTTCTGAAGAGAAA | | 2145 | 8224 | |
| | CAAAGACAACTG | | | 5 | |
| 228 | CAGAACCTTTGCTGCCAGGTGGCTCTTCTGAAGAGAAACAAAGA | 9 | 13578 | 1357 | TSC1 |
| | CAACTGAAGTCAAAGAAATACAGTGTAATCCCTGTAAGTGTAAA | | 2195 | 8229 | |
| | ACTGCTTACACT | | | 5 | |
| 229 | TTCTTAAACACATATAACCCAATTAGAAGAGGCAAGCAAGGCCT | 9 | 13578 | 1357 | TSC1 |
| | GTAGTAACGCAGAAATTTTACCTGATCCTCTGTCATTCAGAAGA | | 2622 | 8272 | |
| | TGGTGTTGTCTG | | | 2 | |
| 230 | ACGCAGAAATTTTACCTGATCCTCTGTCATTCAGAAGATGGTGTT | 9 | 13578 | 1357 | TSC1 |
| | GTCTGTGTAGACATGGTCTTGCAGAATCCATTCTCTCTTCCTGAA | | 2672 | 8277 | |
| | AAGATAAGTA | | | 2 | |
| 231 | TGTAGACATGGTCTTGCAGAATCCATTCTCTCTTCCTGAAAAGAT | 9 | 13578 | 1357 | TSC1 |
| | AAGTATCATTTATATCACAAGACGAAAAATGTTGCACATGTTCT | | 2722 | 8282 | |
| | CGAGCATATTG | | | 2 | |
| 232 | ATCACACCTTGAGAGCAGCTTGTTAGTCCATTTTCAATTATTCTG | 9 | 13578 | 1357 | TSC1 |
| | ATTCAAACCCATTGCATTTTAGGTCAGAATTCTATCTGGCATAAT | | 5792 | 8589 | |
| | TAGGCTTCTC | | | 2 | |
| 233 | AACCCATTGCATTTTAGGTCAGAATTCTATCTGGCATAATTAGGC | 9 | 13578 | 1357 | TSC1 |
| | TTCTCAAAGTGAGGCTTGCAAGTGAGTCACTGTGCCTGGGCAGA | | 5842 | 8594 | |
| | GGGATAGCAGA | | | 2 | |
| 234 | AAAGTGAGGCTTGCAAGTGAGTCACTGTGCCTGGGCAGAGGGA | 9 | 13578 | 1357 | TSC1 |
| | TAGCAGACGAGCTGGATCGCACCTTCCTGGGGGGTGTGACTGTG | | 5892 | 8599 | |
| | GCCTGGGGGAGTG | | | 2 | |
| 235 | CGAGCTGGATCGCACCTTCCTGGGGGGTGTGACTGTGGCCTGGG | 9 | 13578 | 1357 | TSC1 |
| | GGAGTGAAATGTGCACGTAGTCATCCGAATGACAGAGTGGGGC | | 5942 | 8604 | |
| | TGGAGGAGGAGAG | | | 2 | |
| 236 | AAATGTGCACGTAGTCATCCGAATGACAGAGTGGGGCTGGAGG | 9 | 13578 | 1357 | TSC1 |
| | AGGAGAGGTTGCTGGGGTTCCCAGAGGAGTTCCTTTTCCACCTG | | 5992 | 8609 | |
| | CTTAGAGACAAGG | | | 2 | |
| 237 | GTTGCTGGGGTTCCCAGAGGAGTTCCTTTTCCACCTGCTTAGAGA | 9 | 13578 | 1357 | TSC1 |
| | CAAGGGCAGAACATATATGAACACTGAGCCCAACTATTAGAAA | | 6042 | 8614 | |
| | AACTGCCGATTT | | | 2 | |
| 238 | GAGAGCTCCTCCTGCCATTAAAGGCAGGCCAAAACCAACTAATC | 9 | 13578 | 1357 | TSC1 |
| | AAATCCAACCTAAGACATACATACCAGTTGTACCAAAGACTTTA | | 6320 | 8642 | |
| | CTGTAAGGGTGT | | | 0 | |
| 239 | AACCTAAGACATACATACCAGTTGTACCAAAGACTTTACTGTAA | 9 | 13578 | 1357 | TSC1 |
| | GGGTGTGACAGATCAGGTGGGACATTTCCAGGAGAAGTTGGAG | | 6370 | 8647 | |
| | GAGTGGTCATACC | | | 0 | |
| 240 | GACAGATCAGGTGGGACATTTCCAGGAGAAGTTGGAGGAGTGG | 9 | 13578 | 1357 | TSC1 |
| | TCATACCACAAACCATAGATGGGCTCCAAAGAGTAGCCTGGGA | | 6420 | 8652 | |
| | AGTTAATAAAGTAC | | | 0 | |
| 241 | ACAAACCATAGATGGGCTCCAAAGAGTAGCCTGGGAAGTTAAT | 9 | 13578 | 1357 | TSC1 |
| | AAAGTACATCAGCAGTGGCAAAGGAATGCTAAGTCATCCACGA | | 6470 | 8657 | |
| | GGTTTATATCCATG | | | 0 | |
| 242 | GGATCCTTAAAAGTGACTCCTGAAATGAGCAGTGTGAAATTTTC | 9 | 13578 | 1357 | TSC1 |
| | CCAACCACATACTAAATCTGACCCAAAGGGTCAGCTTCACCAGA | | 6712 | 8681 | |
| | AAGCAGAGGAGA | | | 2 | |
| 243 | ACATACTAAATCTGACCCAAAGGGTCAGCTTCACCAGAAAGCAG | 9 | 13578 | 1357 | TSC1 |
| | AGGAGAGAGCAGGCACACTAGTTGACACCATACTTGTGGTGGTT | | 6762 | 8686 | |
| | CAGTTATCAGCC | | | 2 | |
| 244 | GAGCAGGCACACTAGTTGACACCATACTTGTGGTGGTTCAGTTA | 9 | 13578 | 1357 | TSC1 |
| | TCAGCCGTGTCGATGGGGAACTCAGAGTCTGAGGTAGCTGCCCT | | 6812 | 8691 | |
| | GGCATATTTAAC | | | 2 | |
| 245 | GTGTCGATGGGGAACTCAGAGTCTGAGGTAGCTGCCCTGGCATA | 9 | 13578 | 1357 | TSC1 |
| | TTTAACAACATCAGCCGAGACGTGGAGTAAGGGGTAGAAGTAG | | 6862 | 8696 | |
| | CACACCCTAAAAT | | | 2 | |
| 246 | AACATCAGCCGAGACGTGGAGTAAGGGGTAGAAGTAGCACACC | 9 | 13578 | 1357 | TSC1 |
| | CTAAAATGGAAGAGAAGAACACAGGGGGTTAGTGTGTGGTTTT | | 6912 | 8701 | |
| | AGGTTATTCTGGTT | | | 2 | |
| 247 | ACAAATAATGTTTTCCAGAGACAAAGTTGCAAAACAGATAAGTA | 9 | 13578 | 1357 | TSC1 |
| | CCAAAGACACTTTTTACCATAGCTATTCTGTGTGTCAGCATAAG | | 7607 | 8770 | |
| | GGCTGGTGGTGA | | | 7 | |
| 248 | ACACTTTTTACCATAGCTATTCTGTGTGTCAGCATAAGGGCTGGT | 9 | 13578 | 1357 | TSC1 |
| | GGTGACATCGGCTGAACGATGAGGAAAGCGGGCTGAGATTTGG | | 7657 | 8775 | |
| | TGAGACACAGAA | | | 7 | |
| 249 | CATCGGCTGAACGATGAGGAAAGCGGGCTGAGATTTGGTGAGA | 9 | 13578 | 1357 | TSC1 |
| | CACAGAATAGCCATCTTCATATGAGGCTTCTGTGGGATCCAGAG | | 7707 | 8780 | |
| | AGATTTTGGCACA | | | 7 | |
| 250 | TAGCCATCTTCATATGAGGCTTCTGTGGGATCCAGAGAGATTTT | 9 | 13578 | 1357 | TSC1 |
| | GGCACACTCGATCACAACATCATGAGTTTCTAATCTCTTCCACCT | | 7757 | 8785 | |
| | GTAAAATGCAA | | | 7 | |
| 251 | CTCGATCACAACATCATGAGTTTCTAATCTCTTCCACCTGTAAAA | 9 | 13578 | 1357 | TSC1 |
| | TGCAATGAAAGTCAAGAAATGCAAACTGTAATCAACTGAATTAA | | 7807 | 8790 | |
| | ATACTTCAGAG | | | 7 | |
| 252 | CTCCTAGATCACATTTTCAATCTCTCGAAAGATTCTTTAAAATTT | 9 | 13579 | 1357 | TSC1 |
| | TGACACTAGTTTCTATACCTTCGAGGGTCCAGTTCATGGTCCTTG | | 6686 | 9678 | |
| | GATCCAGTCA | | | 6 | |
| 253 | CTAGTTTCTATACCTTCGAGGGTCCAGTTCATGGTCCTTGGATCC | 9 | 13579 | 1357 | TSC1 |
| | AGTCACTAATTCCGGATGAATTCGCACATGCTCCATCATTGGCT | | 6736 | 9683 | |
| | AGAAGAGTTGG | | | 6 | |
| 254 | CTAATTCCGGATGAATTCGCACATGCTCCATCATTGGCTAGAAG | 9 | 13579 | 1357 | TSC1 |
| | AGTTGGGTTGACAAATTATAAAGGGCTGAATGTTTGTGGAACAT | | 6786 | 9688 | |
| | CCAAATGATGGA | | | 6 | |
| 255 | ATACAAAAGGTATAAATGCAGCCTATCTAAACAGTATACTAAGT | 9 | 13579 | 1357 | TSC1 |
| | AGCAAACAAACAAGCAGTTTCAATTTACCTTGACCACTTCTTCA | | 7133 | 9723 | |
| | AAAGTCTCCAGG | | | 3 | |
| 256 | CAAACAAGCAGTTTCAATTTACCTTGACCACTTCTTCAAAAGTCT | 9 | 13579 | 1357 | TSC1 |
| | CCAGGTTTTCTTTCATACTGTAATGAGAACGCAAAAAGGAGACG | | 7183 | 9728 | |
| | AAGTTGCAAGG | | | 3 | |
| 257 | TTTTCTTTCATACTGTAATGAGAACGCAAAAAGGAGACGAAGTT | 9 | 13579 | 1357 | TSC1 |
| | GCAAGGGTACATTCCATAAAGGCGATGAAAGAGTGCGTACACA | | 7233 | 9733 | |
| | CTGGCATGGAGAT | | | 3 | |
| 258 | GTACATTCCATAAAGGCGATGAAAGAGTGCGTACACACTGGCAT | 9 | 13579 | 1357 | TSC1 |
| | GGAGATGGACGAGATAGACTTCCGCCACGTGGCCTAGAAAAGG | | 7283 | 9738 | |
| | AACCCGTTGAGAA | | | 3 | |
| 259 | GGACGAGATAGACTTCCGCCACGTGGCCTAGAAAAGGAACCCG | 9 | 13579 | 1357 | TSC1 |
| | TTGAGAAGAGCCTCTTAGTTGGAGACAGATTGAGGAGTGCAAA | | 7333 | 9743 | |
| | ACAGCTATAAACAA | | | 3 | |
| 260 | AATGAAAGCATTCACCTCACAGGGCCCAACAGGTATATGAGGA | 9 | 13579 | 1357 | TSC1 |
| | GATCTGTACCTGGTTTCTTCAGGCACCATGATGACAGACGGCCA | | 8682 | 9878 | |
| | AAAATGTCAAAGA | | | 2 | |
| 261 | ACCTGGTTTCTTCAGGCACCATGATGACAGACGGCCAAAAATGT | 9 | 13579 | 1357 | TSC1 |
| | CAAAGAAATCAAGAAGATGCTGTTTCCCAGACTGTGGAATCATT | | 8732 | 9883 | |
| | GGTAGCATGGTT | | | 2 | |
| 262 | AATCAAGAAGATGCTGTTTCCCAGACTGTGGAATCATTGGTAGC | 9 | 13579 | 1357 | TSC1 |
| | ATGGTTATCAACACCAAGACGCCTGTTGTGAGGACAACGACGTC | | 8782 | 9888 | |
| | AGTGTCCATCTG | | | 2 | |
| 263 | ATCAACACCAAGACGCCTGTTGTGAGGACAACGACGTCAGTGTC | 9 | 13579 | 1357 | TSC1 |
| | CATCTGCAGGAGAAAAGGTCAAACAGGAAACGTCTGTCAGGCA | | 8832 | 9893 | |
| | CTGGCACCAGGAT | | | 2 | |
| 264 | GCTTTAAGTTGCCTAAAATTTCAGAAACTATACTCATAAAACCA | 9 | 13580 | 1358 | TSC1 |
| | TTTCATTCAAATCCTTACAAACATCCTACCTTGAGACATTTTAGT | | 0900 | 0100 | |
| | AAAGAAGGCAA | | | 0 | |
| 265 | TCAAATCCTTACAAACATCCTACCTTGAGACATTTTAGTAAAGA | 9 | 13580 | 1358 | TSC1 |
| | AGGCAAAAGAGGTGCTTGAGAGAGCTTATGCTTCCAAGATGGCT | | 0950 | 0105 | |
| | GCAGTCTTATGA | | | 0 | |
| 266 | AAGAGGTGCTTGAGAGAGCTTATGCTTCCAAGATGGCTGCAGTC | 9 | 13580 | 1358 | TSC1 |
| | TTATGACATGACCCAGTAACGAGAGGATGGATAAACGAGTGGC | | 1000 | 0110 | |
| | GGCTTTGCCCACA | | | 0 | |
| 267 | CATGACCCAGTAACGAGAGGATGGATAAACGAGTGGCGGCTTT | 9 | 13580 | 1358 | TSC1 |
| | GCCCACATATTCGTTAATCCTGTCCAAGAGGTGCTGAAAATGTA | | 1050 | 0115 | |
| | AAAGAACAAGGGC | | | 0 | |
| 268 | TATTCGTTAATCCTGTCCAAGAGGTGCTGAAAATGTAAAAGAAC | 9 | 13580 | 1358 | TSC1 |
| | AAGGGCAGTCCTCACATGAATGTATGAAGTTAACACAAATAAA | | 1100 | 0120 | |
| | GACAGCAATGATG | | | 0 | |
| 269 | ACAGTGGCCGTGCACAGAAGCTGTTGTACTCATGAAGAACATAT | 9 | 13580 | 1358 | TSC1 |
| | GAAATGCCTATGATATTTCAGCCATTACCTTGTCATGTGGCTCTT | | 2515 | 0261 | |
| | GCAAGGTGGTC | | | 5 | |
| 270 | CCTATGATATTTCAGCCATTACCTTGTCATGTGGCTCTTGCAAGG | 9 | 13580 | 1358 | TSC1 |
| | TGGTCAGGATGTGCAATGCCGGCTGAGAGCTGGTTTCCAGGTAA | | 2565 | 0266 | |
| | TAATCCACCAA | | | 5 | |
| 271 | AGGATGTGCAATGCCGGCTGAGAGCTGGTTTCCAGGTAATAATC | 9 | 13580 | 1358 | TSC1 |
| | CACCAAGGTGTTTACAAGCATAGGGCCACGGTCTAAATCAAGAA | | 2615 | 0271 | |
| | AAGGGCAATGGA | | | 5 | |
| 272 | GGTGTTTACAAGCATAGGGCCACGGTCTAAATCAAGAAAAGGG | 9 | 13580 | 1358 | TSC1 |
| | CAATGGATGATACTTATTCCCCTTAACATCCTAAATTTACCTTAC | | 2665 | 0276 | |
| | ACAGCTTCCTGT | | | 5 | |
| 273 | AGGATTCTAGTGGCTCTAAAGTCAATCTCTTCTTTCTAGAAGATA | 9 | 13580 | 1358 | TSC1 |
| | AGCTAAAAAGGATATTATTTTGCTAACCAGAATTGAGGTTCTCT | | 4081 | 0418 | |
| | TTAAAGACAGC | | | 1 | |
| 274 | AAAAGGATATTATTTTGCTAACCAGAATTGAGGTTCTCTTTAAA | 9 | 13580 | 1358 | TSC1 |
| | GACAGCTGTCACGTCGTCCCGCACACCCAGCATGGGGGAGTCCA | | 4131 | 0423 | |
| | GCATGGCAAGAA | | | 1 | |
| 275 | TGTCACGTCGTCCCGCACACCCAGCATGGGGGAGTCCAGCATGG | 9 | 13580 | 1358 | TSC1 |
| | CAAGAAGCTCCCCGACATTTGCTTGTTGGGCCATTCTCTCGCTCG | | 4181 | 0428 | |
| | AAGGCGCTGTG | | | 1 | |
| 276 | GCTCCCCGACATTTGCTTGTTGGGCCATTCTCTCGCTCGAAGGCG | 9 | 13580 | 1358 | TSC1 |
| | CTGTGCTGGCTCCAGGACGTGTGCTACAGGTTCTGAAGGTTCTTC | | 4231 | 0433 | |
| | ATTGGGGCCA | | | 1 | |

2. Construction of gDNA Library

The library was constructed according to the conventional method:

1) Sampling.

a) DNA concentration of each sample was detected and recorded by Qubit2.0;

b) 50 ng of the sample was taken and placed in a 0.2 ml PCR tube,

2) DNA Fragmentation

a) Samples in the PCR tubes were fragmented according to the following table

| Components | volume (μL) |
|---|---|
| Input Double-stranded DNA | 35 |
| KAPA Frag Buffer (10X) | 5 |
| KAPA Frag Enzyme | 10 |
| Total | 50 |

b) Fragmentation reaction was performed according to the following procedures:

| | | |
|---|---|---|
| Temperature | Time | Heated-lid temperature |

| | | | |
|---|---|---|---|
| 4° C. | 30 | S | 50° C. |
| 37° C. | 20 | min* | |

| | |
|---|---|
| 4° C. | Hold |

3) End Repair and A-Tailing

a) The fragmented samples in the PCR tubes were processed according to the following table

| Components | volume (μL) |
|---|---|
| Fragmented DNA | 50 |
| End Repair & A-Tailing Buffer | 7 |
| End Repair & A-Tailing Enzyme Mix | 3 |
| Total | 60 |

b) The reaction was performed according to the following procedures:

| Temperature | Time | Heated-lid temperature |
|---|---|---|
| 65° C. | 30 min | 85° C. |
| 4° C. | Hold | |

4) Adapter Connection

a) The repaired and a-tailed samples in the PCR tubes were processed according to the following table:

| Components | volume (μL) |
|---|---|
| End repair and A-tailing product | 60 |
| Index Adapter | 2.5 |
| PCR-grade water | 7.5 |
| Ligation Buffer | 30 |
| DNA Ligase | 10 |
| Total | 110 |

b) The reaction was performed according to following procedures:

| Temperature | Time | Heated-lid temperature |
|---|---|---|
| 20° C. | 20 min | 85° C. |
| 4° C. | Hold | |

5) Purification of Ligation Product and Screening Fragment

a) The ligation product was taken out, transferred to a 1.5 mL EP tube containing 88 μL of Ampure xp Beads, mixed well, micro-centrifuged, placed at room temperature for 5 min, and then it was placed on a magnetic stand until the solution became clear to remove the supernatant;

b) 200 μL of freshly prepared 80% ethanol was added to the EP tube which was then rotated several times and the supernatant was removed after the solution became clear, and the tube was slightly dried at room temperature;

c) 50.0 μL of ddH2O was added to the tube and mixed well, micro-centrifuged, placed at room temperature for 5 minutes, and then the tube was placed on a magnetic stand until the solution became clear. Then, the supernatant was transferred to a 1.5 mL EP tube containing 50 μL of Ampure xp Beads, mixed well, micro-centrifuged, and placed at room temperature for 5 minutes, followed by putting the EP tube on the magnetic stand until the solution was clear to remove the supernatant;

d) 200 μL of freshly prepared 80% ethanol was added to the EP tube which was then rotated several times, and the supernatant was removed after the solution was clear;

e) 200 μL of freshly prepared 80% ethanol was added to the EP tube which was then several times, and the supernatant was removed after the solution was clear, and the tube was slightly dried at room temperature;

f) 21 μL ddH2O Elute was used for eluting, and then for later use in the next PCR step.

6) PCR Reaction

a) A new PCR tube was taken to prepare a PCR system according to the following table:

| Components | volume (μL) |
|---|---|
| Adapter-ligated library | 20 |
| KAPA HiFi HotStart ReadyMix (2X) | 25 |
| Library Amplification Primer Mix (10X) | 5 |
| Total | 50 |

b) Amplification was done according to the following reaction procedure:

98° C., 45 s→(98° C., 15 s, 60° C., 30 s, 72° C., 30 s) 8 cycles→72° C., 1 min→4° C., ∞.

7) Purification of PCR Product

a) The PCR product was taken out, transferred to a 1.5 mL EP tube containing 50 μL of Ampure xp Beads, mixed well, micro-centrifuged, placed at room temperature for 10 to 15 min, and then the tube was placed on a magnetic stand until the solution became clear to remove the supernatant;

b) 200 μL of freshly prepared 80% ethanol was added to the EP tube which was then rotated several times, and the supernatant was removed after the solution was clear;

c) 200 μL of freshly prepared 80% ethanol was added to the EP tube which was then rotated several times, and the supernatant was removed after the solution was clear, and the tube was slightly dried at room temperature;

d) 21 μL ddH2O was added to elute the library;

e) The library concentration of each sample was detected and recorded by Qubit2.0;

f) The library fragment size of each sample (Optional) was detected by a 2100 chip analyzer.

3. Hybrid Capture

1) Probe Hybridization

a) DNA library Pooling: According to the concentration measured by Qubit2.0, samples, with an amount of 100 ng for each sample, were mixed in a PCR tube, and 5 samples were subjected to one hybridization reaction;

b) Blocking Oligos were added to the PCR tube of the pooled library,

| Components | volume (μL) |
|---|---|
| Pooled Library (Illumina) | 500 ng |
| Cot-1 DNA | 5 μg |
| xGen ® Universal Blockers-TS Mix | 2 |

c) After well mixing by repetitive pipetting, the mixed solution was dried by a vacuum filtration system (the temperature was set to 60° C.);

d) The following hybridization buffer was added to the dried PCR tube, mixed well by repetitive pipetting, and the tube was placed at room temperature for 5 to 10 minutes:

| Components | Volume (μL) |
|---|---|
| xGen 2X Hybridization Buffer | 8.5 |
| xGen Hybridization Buffer Enhancer | 2.7 |
| Nuclease-Free Water | 1.8 |

e) The PCR tube was placed on a PCR machine, incubated at 95° C. for 10 minutes to denature;

f) The PCR tube was taken out immediately after the denaturation, and then it was placed on a pre-cooled metal plate, and 4 μL of xGen Lockdown Probe pool (probe) was immediately added;

g) Hybridization was performed according to the following procedure:

| Temperature | Time | Heated-lid temperature |
|---|---|---|
| 65° C. | >14 h | 75° C. |

2) Capture Elution

a) Each wash Buffer was diluted in the following proportions (each one is satisfied with the amount of one capture elution):

| | volume(μl) | | total volume |
|---|---|---|---|
| | of Stock | volume(μl) | of 1X |
| Reagent Name | solution | of ddH₂O | Buffer (μl) |
| xGen 10X Wash Buffer I | 30 | 270 | 300 |
| xGen 10X Wash Buffer II | 20 | 180 | 200 |
| xGen 10X Wash Buffer III | 20 | 180 | 200 |
| xGen 10X Stringent Wash | 40 | 360 | 400 |
| Buffer | | | |
| xGen 2X Bead Wash Buffer | 250 | 250 | 500 |

b) 400 μl of 1×Stringent Wash Buffer was pre-heated in a 65° C. metal warm bath;

c) 100 μl of 1×Wash Buffer I was aliquoted and placed in the 65° C. warm bath to preheat, and the remaining 20010 of 1×Wash Buffer I was placed at room temperature for use;

d) Dynabeads® M-270 Streptavidin beads were taken out from 4° C. and warmed to room temperature. After enough shaking, 100 μl of the solution was pipetted into a 1.5 ml centrifuge tube, which was then placed on a magnetic stand to remove the supernatant after the solution was clear;

e) The centrifuge tube was taken out from the magnetic stand, 20010 of 1×Bead Wash Buffer was added into the centrifuge tube, which was shaken for 10 s, put back on the magnetic stand after microcentrifugation, and then the supernatant was removed after the solution was clear;

f) The above steps were repeated once;

g) 100 μl of 1×Bead Wash Buffer was added for resuspending Dynabeads® M-270 Streptavidin beads, transferred to a new 20010 PCR tube, the tube was placed on the magnetic stand, the supernatant was removed for later use after the solution became clear;

h) The samples that were hybridized overnight (the program of the PCR instrument was maintained at 65° C. and the heated lid was kept at 75° C.) were taken out, all the liquid was transferred to the Dynabeads®M-270 Streptavidin beads washed in the previous step, mixed well by repetitive pipetting, and placed back to the PCR machine again after micro-centrifugation;

i) After a 12 min-reaction, the samples were taken out and mixed 3 times;

j) After the reaction was completed, the PCR tube was taken out, 10010 of 1×Wash Buffer I (preheated at 65° C.) was added, fully shaken for 10 s, and then the liquid was transferred to a 1.5 ml centrifuge tube, which was placed on the magnetic stand, and the supernatant was removed after the solution became clear;

k) The centrifuge tube was taken out from the magnetic stand, 20010 of 1×Stringent Buffer (preheated at 65° C.) was added and mixed well, and the tube was quickly put back into the 65° C. water bath, and warmed for 5 minutes;

l) The step k) was repeated once;

m) After warming in the warm bath, the tube was placed on the magnetic stand, and the supernatant was removed after the solution became clear;

n) The centrifuge tube was taken out from the magnetic stand, 20010 of 1×Wash Buffer I (room temperature) was added, shaken for 2 minutes, placed on the magnetic frame after microcentrifugation, and the supernatant was removed after the solution became clear;

o) The centrifuge tube was taken out from the magnetic stand, 20010 of 1×Wash Buffer II was added, shaken for 1 min, placed on the magnetic stand after microcentrifugation, and the supernatant was removed after the solution became clear;

p) The centrifuge tube was taken out from the magnetic stand, 20010 of 1×Wash Buffer III was added, shaken for 30 s, and placed on the magnetic stand after microcentrifugation, and the supernatant was removed after the solution became clear;

q) 20 μl of ddH2O was added to resuspend and elute Dynabeads® M-270 Streptavidin beads for PCR.

3) Second PCR (Post-PCR)

a) A new PCR tube was taken to prepare a PCR system according to the following table:

| System Components | Volume (μl) |
|---|---|
| Capture elution product from the previous step | 20 |
| KAPA HiFi HotStart ReadyMix | 25 |
| 10 μM Illumina P5 primer | 2.5 |
| 10 μM Illumina P7 primer | 2.5 |
| Total volume | 50 |

b) The library was amplified according to the following reaction procedure:

98° C., 45 s→(98° C., 15 s, 60° C., 30 s, 72° C., 30 s) 11 cycles→72° C., 1 min→4° C., ∞.

4) Purification for PCR Product

a) The PCR product was taken out, transferred to a 1.5 mL EP tube containing 750, of Ampure xp Beads, mixed well, micro-centrifuged, and placed at room temperature for 10 to 15 min, and then the EP tube was placed on the magnetic stand until the solution became clear to remove the supernatant;

b) 200 μL of freshly prepared 80% ethanol was added to the EP tube, rotated several times, and the supernatant was removed after the solution became clear;

c) 200 μL of freshly prepared 80% ethanol was added to the EP tube, rotated several times, and the supernatant was removed after the solution became clear, and the tube was dried at room temperature;

d) 21.0 μL of ddH2O was added to elute the library.

5) Quality Control for Library

a) The concentration of the final library was measured by Qubit 2.0;

b) Library fragment sizes (Optional) were detected by Agilent 2100 Bioanalyzer;

6) Sequencing on the machine

Illumina Nextseq500 was used.

II. Sorting.

The above sequencing data were processed and analyzed by bioinformatics. If TSC1 and TSC2 genes 0 were detected to be negative or there was only one-hit mutated locus, a supplementary detection was performed by the chromosomal microarray analysis and the Multiplex ligation-dependent probe amplification. When a locus was detected to be an undefined locus originated from either somatic mutation or the germline mutation, the locus can be verified by Sanger sequencing.

III. Performing Chromosomal Microarray Analysis and Multiplex Ligation-Dependent Probe Amplification.

If TSC1 and TSC2 genes were detected to be negative or there was only a one-hit mutated locus (that is, gene mutation, or fragment deletion/insertion, or copy number variations, etc. only occurred in one of the TSC1 gene and TSC2 gene), a supplementary detection was performed in the chromosome microarray analysis and Multiplex ligation-dependent probe amplification. The agents used were as follows.

| Analysis | | | Article |
|---|---|---|---|
| method | Name of Agents | manufacture | Number |
| CMA | OncoScan ® CNV FFPE | Affymetrix | 902695 |
| | Assay Kit | | |
| MLPA | TSC1&TSC2 Probe | MRC-Holland | P124&P046 |

IV. Performing Sanger Sequencing.

If a locus was detected to be an undefined locus derived from either a somatic mutation or a germline mutation, specifically, the undefined locus derived from either a somatic mutation or a germline mutation was referred to a variant with a mutation frequency of about 50%. The patients leukocyte specimen (ABI 3730XL) was verified by Sanger sequencing, and it can be identified whether the patient belonged to S-LAM or TSC-LAM through the validation results.

If the leukocytes of the patient also had the above mutations, it can be indicated that the patient belongs to TSC-LAM; otherwise, it can be indicated that the patient belongs to S-LAM.

### Example 2

The joint detection for LAM was studied with the method of Example 1.

1. Comparison of Single Detection Method and Joint Detection Method.

In this study, a total of 61 LAM patients were employed in a group, and a single detection method and the method of Example 1 were used for detection at the same time.

The single detection method was a method that only used NGS, a Target Sequencing based Hybridization capture.

The results were shown in FIGS. 3 to 4. FIG. 3 showed the detection ratio of variants by using different detection methods, wherein NGS meant Target Sequencing based Hybridization capture, CMA meant chromosome microarray analysis, and MLPA meant multiplex ligation-dependent probe amplification detection. The above results showed that, SNV, INDEL, CNV, LOH and other variant types 0 can be comprehensively detected for LAM patient samples by the multi-method joint detection method, while 12.8% of the variants would be lost when only single NGS detection method was used.

When a single detection method was used, the overall positive detection rates for TSC1 and TSC2 genes were 72.13%, and there were 15 patients detected with 1-hit, and 29 patients with 2-hits, respectively. When the joint detection scheme was used, the overall positive detection rate was 75.41%, and 1-hit was 5 detected in 8 patients, and 2-hits were detected in 38 patients, respectively. The results were shown in FIG. 4.

The joint detection scheme not only improves the detection rate of positive mutations in LAM patients, but more importantly, the detection results are more in line with Knudson's “double hit” theory.

2. Research Findings.

When the joint detection method of Example 1 was used, 30 new mutations were found in 61 LAM patients employed in the group, as follows:

| | | | Mutation |
|---|---|---|---|
| Gene HGVS(cDNA) | HGVS(protein) | Exon | type |
| TSC2 NM_000548.4: c.2220 + 2_2220 + 9del | N/A | splice | Deletion |
| TSC1 NM_000368.4: c.979C > T | p.Pro327Ser | exon10 | SNV |
| TSC2 NM_000548.4: c.3059_3063del | p.Leu1020Profs*146 | exon27 | Deletion |
| TSC2 NM_000548.4: c.430A > T | p.Lys144* | exon5 | SNV |
| TSC2 NM_000548.4: c.1808C > G | p.Thr603Ser | exon17 | SNV |
| TSC2 NM_000548.4: c.1221C > G | p.Tyr407* | exon12 | SNV |
| TSC2 NM_000548.4: c.5106delC | p.Ile1702Metfs*124 | exon40 | Deletion |
| TSC2 NM_000548.4: c.781C > G | p.Arg261Gly | exon9 | SNV |
| TSC2 NM_000548.4: c.5004delT | p.Phe1668Leufs*4 | exon39 | Deletion |
| TSC2 NM_000548.4: c.1118A > G | p.Gln373Arg | exon11 | SNV |
| TSC2 NM_000548.4: c.4933_4937delinsACAATGTCTACAATGTCTACA | p.Phe1645Thrfs*13 | exon38 | Insertion |
| TSC2 NM_000548.4: c.2220 + 2_2220 + 9del | N/A | splice | Deletion |
| TSC2 NM_000548.4: c.3755C > T | p.Ser1252Leu | exon31 | SNV |
| TSC2 NM_000548.4: c.151delG | p.Glu51Asnfs*10 | exon3 | Deletion |
| TSC2 NM_000548.4: c.l714delC | p.Gln572Argfs*126 | exon16 | Deletion |
| TSC2 NM_000548.4: c.460_461del | p.Thr154Leufs*8 | exon5 | Deletion |
| TSC2 NM_000548.4: c.2098-1G > C | N/A | splice | SNV |
| TSC2 NM_000548.4: c.25T > C | p.Ser9Pro | exon2 | SNV |
| TSC2 NM_000548.4: c.400G > T | p.Glu134* | exon5 | SNV |
| TSC2 NM_000548.4: c.219_223del | p.Phe73Leufs*51 | exon3 | Deletion |
| TSC2 NM_000548.4: c.4682delT | p.Ile1561Thrfs*15 | exon37 | Deletion |
| TSC2 NM_000548.4: c.3500_3507del | p.Glu1167Valfs*64 | exon30 | Deletion |
| TSC2 NM_000548.4: c.788delT | p.Leu263Profs*30 | exon9 | Deletion |
| TSC2 NM_000548.4: c.2267delG | p.Gly756Alafs*15 | exon21 | Deletion |
| TSC2 NM_000548.4: c.3132delG | p.Arg1044Serfs*9 | exon28 | Deletion |
| TSC2 NM_000548.4: c.1829T > A | p.Ile610Asn | exon17 | SNV |
| TSC2 NM_000548.4: c.5116_5119dupCGCA | p.Asn1707Thrfs*23 | exon40 | Insertion |
| TSC2 NM_000548.4: c.2153_2154del | p.Arg718Leufs*2 | exon20 | Deletion |
| TSC2 NM_000548.4: c.2018C > G | p.Ala673Gly | exon19 | SNV |
| TSC1 NM_000368.4: c.1450A > G | p.Arg484Gly | exon15 | SNV |

These new mutations will greatly enrich the genetic database of LAM, a rare disease, and have important clinical value in promoting the research progress and treatment guidance of the disease.

### Example 3

An example of the joint detection for LAM was conducted with the method of Example 1.

### I. Sample Source

The samples were obtained from fixed tissue samples from the Department of Respiratory Medicine of a tertiary hospital in Guangzhou, and they were clinically diagnosed as S-LAM.

### II. Detection Methods and Results

1. Target Sequencing Based Hybridization Capture (Target Capture Sequencing, a NGS) was Performed with Probes

The sequencing results showed that the sample had a single mutated locus, which is TSC2: NM_000548.4:c.3412C>T (p.Arg1138*), and the variant frequency was 50.9%.

2. Chromosome Microarray Analysis and Multiplex Ligation-Dependent Probe Amplification

According to the method of Example 1, a supplementary detection was performed by the chromosome microarray analysis. The results showed that there was a loss of heterozygosity (LOH) variation in the TSC2 gene of the patient: arr<GRCh37>16p13.3p11.2(83886_30809063)x2 mos hmz. The size of the variant was 30.73 Mb, and its LOH fragment was shown in FIG. 5; the detection result was negative when the multiplex ligation probe amplification was used.

3. Sanger Sequencing

Since the variant frequency of the locus detected by NGS was 50.9%, it was impossible to determine whether the locus was derived from a somatic mutation or a germline mutation. Therefore, primers were designed for such locus and the leukocyte samples of the patient were sequenced. The verification results showed that there was no above-mentioned mutation in leukocytes of the patient, and therefore the mutation was a somatic mutation, that is, sporadic LAM (S-LAM).

### III. Conclusion

The detection results of the patient showed that a premature stop codon was created at codon 1138 of the TSC2 gene, with a mutation frequency of 50.9%. It was determined by the Sanger method that the mutation did not exist in leukocytes and was a somatic mutation, leading to normal protein disfunction. In the past, this mutation had been reported for more than 50 times in individuals with LAM, and it was a pathogenic mutation. The detection results of the supplementary detection showed that the patient also had the LOH phenomenon of the TSC2 gene. Studies have shown that LOH can lead to the inactivation of the suppressor genes, thereby affecting the occurrence and development of the tumor. The pathogenesis of the patient can be completely known through this joint detection scheme, and according to the results, sporadic LAM is diagnosed, without hereditary.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A joint detection method for lymphangioleio-myomatosis, comprising:
performing Target Sequencing based Hybridization capture, wherein a Panel is configured for whole coding regions of TSC1 genes and TSC2 genes highly related to lymphangioleio-myomatosis and mutation genes closely related to solid tumors to construct a Genomic DNA library, a gDNA library, and sequencing is performed on a machine after a hybrid capture;
sorting, wherein data obtained from the Target Sequencing based Hybridization capture is processed and analyzed through bioinformatics; when the TSC1 and TSC2 genes are detected to be negative or there is only a one-hit mutated locus, a supplementary detection is performed by chromosomal microarray analysis and Multiplex ligation-dependent probe amplification; when a locus is detected to be an undefined locus originated from either a somatic mutation or a germline mutation, the locus is verified by Sanger sequencing;
performing chromosomal microarray analysis to obtain loss of heterozygosity and copy number variations;
performing multiplex ligation-dependent probe amplification to obtain large fragment insertions and deletions; and
performing Sanger sequencing to test a leukocyte sample corresponding to a sample to be tested after taking the leukocyte sample, and then determining whether the sample is S-LAM or TSC-LAM.

2. The joint detection method for lymphangioleio-myomatosis of claim 1, wherein in the step of performing Target Sequencing based Hybridization capture, the Panel is configured to cover following genes: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

3. The joint detection method for lymphangioleio-myomatosis of claim 1, wherein in the step of performing Target Sequencing based Hybridization capture, probe sequences for the TSC1 and TSC2 genes comprises SEQ ID NO: 1 to SEQ ID NO: 276.

4. The joint detection method for lymphangioleio-myomatosis of claim 1, wherein in the step of performing Target Sequencing based Hybridization capture, a sequencing depth is more than 1000×.

5. A method of investigating a pathogenesis of Lymphangioleio-myomatosis and/or diagnosing and treating Lymphangioleio-myomatosis, comprising applying the joint detection method for Lymphangioleio-myomatosis of claim 1.

6. The method of claim 5, wherein a specific detection reagent in the detection method is applied in a preparation of a diagnostic reagent or a diagnostic equipment for jointly detecting Lymphangioleio-myomatosis.

7. A joint detection kit for Lymphangioleio-myomatosis, comprising a Panel covering genes selected from a group consisting of: ALDH1 gene, EGFR gene, FLT3 gene, MYC gene, PTEN gene, 0 SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

8. The joint detection kit for Lymphangioleio-myomatosis of claim 7, wherein probe sequences of the panel include SEQ ID NO. 1 to SEQ ID NO 276.

9. The joint detection kit for Lymphangioleio-myomatosis of claim 7, wherein the joint detection kit further comprises an agent for chromosomal microarray analysis.

10. The joint detection kit for Lymphangioleio-myomatosis of claim 7, wherein the joint detection kit further comprises multiplex ligation-dependent probes for Multiplex ligation-dependent probe amplification.

11. A joint detection system for Lymphangioleio-myomatosis, comprising:
a detection module, comprising a module of Target Sequencing based Hybridization capture, a module of chromosomal microarray analysis, a module of Multiplex ligation-dependent probe amplification, and a module of Sanger sequencing, wherein the module of the Target Sequencing based Hybridization capture comprises a Panel configured for whole coding regions of TSC1 and TSC2 genes highly related to Lymphangioleio-myomatosis and mutated genes closely related to a solid tumor; and
an analysis module, configured for obtaining a detection result of the Target Sequencing based Hybridization capture; requesting the module of chromosomal microarray analysis and the module of Multiplex ligation-dependent probe amplification to perform a supplementary detection, when TSC1 and TSC2 genes are detected to be negative or there is only a one-hit mutated locus; requesting the module of Sanger sequencing to verify the undefined locus, when a locus is detected to be an undefined locus originated from either a somatic mutation or a germline mutation; and then analyzing and judging detection results from the detection modules, to draw a joint detection result of Lymphangioleio-myomatosis.

12. The joint detection system for Lymphangioleio-myomatosis of claim 11, wherein the Panel covers following genes: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF 5 gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

13. The joint detection system for Lymphangioleio-myomatosis of claim 11, wherein in the module of the Target Sequencing based Hybridization capture, probe sequences for the TSC1 and TSC2 genes comprise SEQ ID NO: 1 to SEQ ID NO: 276.

14. The method of claim 5, wherein in the step of performing Target Sequencing based Hybridization capture, the Panel is configured to cover following genes: ALDH1 gene, EGFR gene, FLT3 gene, MYC gene, PTEN gene, SDHD gene, AQP9 gene, ERBB2 gene, HRAS gene, MYCN gene, RET gene, TP53 gene, AR gene, ESR1 gene, KIT gene, NF1 gene, RICTOR gene, TSC1 gene, ATRX gene, FGFR1 gene, KRAS gene, NRAS gene, RUNX1 gene, TSC2 gene, BCL2 gene, FGFR2 gene, MDM2 gene, PDGFRA gene, SDHA gene, VHL gene, BRAF gene, FGFR3 gene, MAP2K1 gene, PGR gene, SDHB gene, CCND1 gene, FGFR4 gene, MET gene, POLE gene, SDHC gene; ABL1 gene, CDKN2A gene, FBXW7 gene, IDH2 gene, NOTCH1 gene, SMAD4 gene, AKT1 gene, CSF1R gene, GNA11 gene, JAK2 gene, NPM1 gene, SMARCB1 gene, ALK gene, CTNNB1 gene, GNAQ gene, JAK3 gene, PIK3CA gene, SMO gene, APC gene, DDR2 gene, GNAS gene, KDR gene, PTPN11 gene, SRC gene, ATM gene, ERBB4 gene, HNF1A gene, MLH1 gene, RB1 gene, STK11 gene, CDH1 gene, EZH2 gene, IDH1 gene, MPL gene, ROS1 gene, and TET2 gene.

15. The method of claim 5, wherein in the step of performing Target Sequencing based Hybridization capture, probe sequences for the TSC1 and TSC2 genes comprises SEQ ID NO: 1 to SEQ ID NO: 276.

16. The method of claim 5, wherein in the step of performing Target Sequencing based Hybridization capture, a sequencing depth is more than 1000×.
